SEQUENCE LISTING (33 sequences)

SEQ ID NO: 1 - DNA, 20 bp, Artificial Sequence
  Description of Artificial Sequence Synthetic primer
gaagccaaggggaatgaagt

SEQ ID NO: 2 - DNA, 20 bp, Artificial Sequence
  Description of Artificial Sequence Synthetic primer
gggaagggcatttgaagaac

SEQ ID NO: 3 - DNA, 18 bp, Artificial Sequence
  Description of Artificial Sequence Synthetic primer
ctgagtccgcagcaggtg

SEQ ID NO: 4 - DNA, 21 bp, Artificial Sequence
  Description of Artificial Sequence Synthetic primer
tgcccaacaggatatcagact

SEQ ID NO: 5 - DNA, 20 bp, Artificial Sequence
  Description of Artificial Sequence Synthetic primer
cacagtggtgcctaccaaga

SEQ ID NO: 6 - DNA, 21 bp, Artificial Sequence
  Description of Artificial Sequence Synthetic primer
tgattgtcttttgtcaggggt

SEQ ID NO: 7 - DNA, 20 bp, Artificial Sequence
  Description of Artificial Sequence Synthetic primer
ttctacacacccaagacccg

SEQ ID NO: 8 - DNA, 17 bp, Artificial Sequence
  Description of Artificial Sequence Synthetic primer
caatgccacgcttctgc

SEQ ID NO: 9 - DNA, 109 bp, Unknown
  Description of Unknown HNF1A mRNA oligonucleotide
ccggccttgttctgtcaccaatgtactcgagtacattggtgacagaacaaggtttttactcccatgaagacgcagaactcgagttctgcgtcttcatgggagtgttttt

SEQ ID NO: 10 - DNA, 26 bp, Artificial Sequence
  Description of Artificial Sequence Synthetic primer
ccgctcgagcggtgcatcttccagct

SEQ ID NO: 11 - DNA, 26 bp, Artificial Sequence
  Description of Artificial Sequence Synthetic primer
cccaagcttgggcaccttccctgcct

SEQ ID NO: 12 - DNA, 32 bp, Artificial Sequence
  Description of Artificial Sequence Synthetic primer
ccgctcgagcgggctggaatcaatttcccaga

SEQ ID NO: 13 - DNA, 30 bp, Artificial Sequence
  Description of Artificial Sequence Synthetic primer
cggaattccgcgtgatgctgttccagagaa

SEQ ID NO: 14 - DNA, 32 bp, Artificial Sequence
  Description of Artificial Sequence Synthetic primer
ccgctcgagcggtccccaagacacttccacat

SEQ ID NO: 15 - DNA, 30 bp, Artificial Sequence
  Description of Artificial Sequence Synthetic primer
ggactagtccataggcgttccactgacagg

SEQ ID NO: 16 - DNA, 20 bp, Artificial Sequence
  Description of Artificial Sequence Synthetic primer
gcatcttccagctcttcgac

SEQ ID NO: 17 - DNA, 20 bp, Artificial Sequence
  Description of Artificial Sequence Synthetic primer
ctaaagcgcatgctccagac

SEQ ID NO: 18 - DNA, 19 bp, Artificial Sequence
  Description of Artificial Sequence Synthetic primer
aggccctagtttcccatcc

SEQ ID NO: 19 - DNA, 20 bp, Artificial Sequence
  Description of Artificial Sequence Synthetic probe
tccagatgctcctgtcagtg

SEQ ID NO: 20 - DNA, 20 bp, Artificial Sequence
  Description of Artificial Sequence Synthetic probe
gagccaaagcaattccacat

SEQ ID NO: 21 - DNA, 20 bp, Artificial Sequence
  Description of Artificial Sequence Synthetic primer
ttctacacacccaagacccg

SEQ ID NO: 22 - DNA, 17 bp, Artificial Sequence
  Description of Artificial Sequence Synthetic primer
caatgccacgcttctgc

SEQ ID NO: 23 - DNA, 20 bp, Artificial Sequence
  Description of Artificial Sequence Synthetic primer
ctgaacctcaaaccccaaac

SEQ ID NO: 24 - DNA, 20 bp, Artificial Sequence
  Description of Artificial Sequence Synthetic primer
tgccaggatctgctctacct

SEQ ID NO: 25 - DNA, 20 bp, Artificial Sequence
  Description of Artificial Sequence Synthetic primer
catgtgccacactcacacaa

SEQ ID NO: 26 - DNA, 20 bp, Artificial Sequence
  Description of Artificial Sequence Synthetic primer
atccaaactggaaggaaccc

SEQ ID NO: 27 - DNA, 37 bp, Unknown
  Description of Unknown Sequence in intron 7 of GCK
cgtcaataccgagtggggcgccttcggggactccggc

SEQ ID NO: 28 - DNA, 20 bp, Artificial Sequence
  Description of Artificial Sequence Synthetic primer
ctgaacctcaaaccccaaac

SEQ ID NO: 29 - DNA, 20 bp, Artificial Sequence
  Description of Artificial Sequence Synthetic primer
tgccaggatctgctctacct

SEQ ID NO: 30 - DNA, 17 bp, Artificial Sequence
  Description of Artificial Sequence Synthetic primer
atggagcccagcagcaa

SEQ ID NO: 31 - DNA, 20 bp, Artificial Sequence
  Description of Artificial Sequence Synthetic primer
actcctcgatcaccttctgg

SEQ ID NO: 32 - DNA, 20 bp, Artificial Sequence
  Description of Artificial Sequence Synthetic primer
catgtgccacactcacacaa

SEQ ID NO: 33 - DNA, 20 bp, Artificial Sequence
  Description of Artificial Sequence Synthetic primer
atccaaactggaaggaaccc

(19) United States
(12) Patent Application Publication
(10) Pub. No.: US 2014/0242038 A1
(43) Pub. Date: Aug. 28, 2014

(54) METHOD FOR GENERATING BETA CELLS

(71) Applicant: The Trustees of Columbia University in the City of New York, New York, NY (US)

(72) Inventors: Haiqing HUA, New York, NY (US); Dieter EGLI, New York, NY (US); Rudolph L. LEIBEL, New York, NY (US); Linshan SHANG, Fort Lee, NJ (US)

(73) Assignee: The Trustees of Columbia University in the City of New York, New York, NY (US)

(21) Appl. No.: 14/158,481

(22) Filed: Jan. 17, 2014

Related U.S. Application Data
(63) Continuation-in-part of application No. 13/649,040, filed on Oct. 10, 2012.
Application No. 13/649,040 is a continuation-in-part of application No. PCT/US2012/059620, filed on Oct. 10, 2012.
(60) Provisional application No. 61/545,915, filed on Oct. 11, 2011.
(60) Provisional application No. 61/545,915, filed on Oct. 11, 2011.
(60) Provisional application No. 61/753,835, filed on Jan. 17, 2013.
(60) Provisional application No. 61/835,967, filed on Jun. 17, 2013.

(51) Int. Cl. C12N 5/071

(52) U.S. Cl. CPC C12N 5/0676
USPC 424/93.7; 435/377; 435/366; 435/455

(57) ABSTRACT

The invention is directed to methods for generating pancreatic progenitor cells, insulin producing cells or endoderm cells using embryonic stem cells and induced pluripotent stem cells. The present invention also relates to an isolated population comprising pancreatic progenitor cells or a insulin-producing cells, compositions and their use in the treatment of diabetes

This application is a Continuation-In-Part of International Patent Application No. PCT/US2012/059620, filed Oct. 10, 2012, which claims priority of U.S. Provisional Patent Application No. 61/545,915, filed Oct. 11, 2011. This application is a Continuation-In-Part of U.S. patent application Ser. No. 13/649,040, filed Oct. 10, 2012, which claims priority of U.S. Provisional Patent Application No. 61/545,915, filed Oct. 11, 2011. This application claims priority to U.S. Provisional Patent Application No. 61/835,967, filed Jun. 17, 2013 and U.S. Provisional Patent Application No. 61/753,835, filed Jan. 17, 2013, each of which is incorporated herewith in its entirety.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

All publications, patent applications, patents, and other references mentioned herein are incorporated by reference in their entirety. The patent and scientific literature referred to herein establishes knowledge that is available to those skilled in the art. The issued patents, applications, and other publications that are cited herein are hereby incorporated by reference to the same extent as if each was specifically and individually indicated to be incorporated by reference. In the case of inconsistencies, the present disclosure will prevail.

### BACKGROUND

Diabetes can result from gene mutations that affect beta cell development and/or function. Understanding the molecular bases for these distinctive phenotypes can elucidate critical aspects of beta cell biology. However, access to affected human beta cells is limited. There is a need for stem cell technologies to allow for generation of such cells in vitro. This invention addresses this need.

The invention is also generally directed to protein folding and more specifically to methods of treating diseases associated with endoplasmic reticulum stress (ER), including diabetes.

### SUMMARY OF THE INVENTION

In certain aspects, the invention relates to a method for generating a beta cell from a stem cell or an induced pluripotent stem cell, the method comprising: (a) contacting the cells with a first culture medium, wherein the first culture medium is an RPMI medium comprising 1× Pen-Strep and 1× Glutamax and wherein the first culture medium further comprises Activin A, Wnt3A and Ethylene glycol-bis(2-aminoethylether)-N,N,N′,N′-tetraacetic acid, (b) contacting the cells with a second culture medium, wherein the second culture medium is an RPMI medium comprising 1× Pen-Strep and 1× Glutamax and wherein the second culture medium further comprises Activin A protein and FBS in RPMI medium, (c) contacting the cells with a third culture medium, wherein the third culture medium is an RPMI medium comprising 1× Pen-Strep and 1× Glutamax and wherein the third culture medium further comprises containing human FGF10 protein, KAAD-cyclopamine and FBS in RPMI medium, (d) contacting the cells with a fourth culture medium, wherein the fourth culture medium is an DMEM high glucose medium comprising 1× Pen-Strep and 1× Glutamax and wherein the fourth culture medium further comprises FGF10, KAAD-cyclopamine, retinoic acid, LDN-193189 and 1×B27, (e) contacting the cells with a fifth culture medium, wherein the fifth culture medium is a CMRL medium comprising 1× Pen-Strep and 1× Glutamax and wherein the fourth culture medium further comprises exedin-4, SB431542 and 1×B27, and (f) contacting the cells with a sixth culture medium, wherein the sixth culture medium is a CMRL medium comprising 1× Pen-Strep and 1× Glutamax and wherein the sixth culture medium further comprises 4-(4-Hydroxy-3,5-diiodophenoxy)-3,5-diiodobenzeneacetic acid and 1×B27.

In certain embodiments, the beta cell is a pancreatic progenitor cell, an insulin producing cell or an endoderm cell. In certain embodiments, the stem cell is an embryonic stem cell. In certain embodiments, the cells are mammalian cells. In certain embodiments, the cells are human cells.

In certain embodiments, any of the first, second, third, fourth, fifth or sixth culture media further comprise EGTA.

In certain embodiments, the concentration of Activin A in the first culture medium is about 100 ng/ml. In certain embodiments, the concentration of Wnt3A in the first culture medium is about 25 ng/ml. the concentration of Ethylene glycol-bis(2-aminoethylether)-N,N,N′,N′-tetraacetic acid in the first culture medium is about 0.15 mM. In certain embodiments, the cells are cultured in the first culture medium for about 24 hours.

In certain embodiments, the concentration of Activin A in the second culture medium is about 100 ng/ml. In certain embodiments, the concentration of FBS in the second culture medium is about 0.2% FBS by volume. In certain embodiments, the cells are cultured in the second culture medium for about 24 hours.

In certain embodiments, the concentration of FGF10 in the third culture medium is about 50 ng/ml. In certain embodiments, the concentration of KAAD-cyclopamine in the third culture medium is about 0.25 uM. In certain embodiments, the concentration of FBS in the third culture medium is about 2% FBS by volume. In certain embodiments, the cells are cultured in the third culture medium for about 48 hours.

In certain embodiments, the concentration of FGF10 in the fourth culture medium is about 50 ng/ml. In certain embodiments, the concentration of KAAD-cyclopamine in the fourth culture medium is about 0.25 uM. In certain embodiments, the concentration of retinoic acid in the fourth culture medium is about 2 uM. In certain embodiments, the concentration of LDN-193189 in the fourth culture medium is about 250 nM. In certain embodiments, the cells are cultured in the fourth culture medium for about 72 hours.

In certain embodiments, the concentration of exedin-4 in the fifth culture medium is about 50 ng/ml. In certain embodiments, the concentration of SB431542 in the fifth culture medium is about 2 uM. In certain embodiments, the cells are cultured in the fifth culture medium for about 48 hours.

In certain embodiments, the concentration of 4-(4-Hydroxy-3,5-diiodophenoxy)-3,5-diiodobenzeneacetic acid in the sixth culture medium is about 20 pM. In certain embodiments, the cells are cultured in the sixth culture medium for about 48 hours.

In certain embodiments, any of the first, second, third, fourth, fifth or sixth culture media are replaced with fresh corresponding media prior to contacting the cells with media having a different composition.

In certain embodiments, the method further comprises a step of maintaining the cells after step (f) in a CMRL medium comprising 1×B27 and 1× Glutamax.

In certain embodiments, any of the first, second, third, fourth, fifth or sixth culture media further comprise an antibiotic. In certain embodiments, the antibiotic is Pen-Strep.

In certain embodiments, the induced pluripotent cells are generated by (a) obtaining a source cell by taking a skin biopsy from a mammal (e.g. a mouse or a human), (b) establishing a fibroblast cell line from the skin biopsy, and (c) infecting the fibroblast cell line with a retrovirus or a sendai virus capable of directing expression of human transcription factors Oct4, Sox2, Klf4 and C-Myc in the fibroblast cell line.

In certain embodiments, the stem cell or an induced pluripotent stem cell is from a mammal having, or at risk of having, type I diabetes, type II diabetes, pre-diabetes or any combination thereof. In certain embodiments, the stem cell or the induced pluripotent stem cell comprises a diabetes-associated mutation. In certain embodiments, the diabetes-associated mutation is a glucokinase G299R mutation.

In certain aspects, the invention relates to a method for treating a mammal having, or at risk of having, type I diabetes, type II diabetes, pre-diabetes or any combination thereof, the method comprising administering to the mammal a pancreatic progenitor cell, an insulin producing cell or an endoderm cell of claim 1.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-D. Skin fibroblast cells with mutation (G299R) in the glucokinase gene are converted into pluripotent stem cells (FIG. 1A). Quantitative real-time PCR analysis is used to assess the silencing of viral transgenes in the iPS cell lines. All cell lines selected for further characterization and experiments show very low or undetectable levels of viral transgene expression (FIG. 1B). These iPS cells were karyotypically normal (FIG. 1C). They also express specific pluripotency marker genes including Nanog, Tra1-60, SSEA4 (FIG. 1D). iPS cells can spontaneously different into cell types and tissue structures representing all three germ layers both in vitro (embryonic bodies) and in vivo (teratomas) (FIG. 1D).

FIGS. 2A-C. Induced pluripotent stem cells are differentiated into pancreatic progenitors and insulin producing cells (FIG. 2A). EGTA increases the efficiency of generating pancreatic progenitor cells (FIG. 2B). Exendin-4 and SB431542 together greatly improves the efficiency of generating insulin-producing cells (FIG. 2C).

FIGS. 3A-B. Cells are treated with physiological concentrations of glucose (5.6 mM) and subsequently high concentrations of glucose (20 mM). Compared to beta cells derived from human ES cells and control iPS cells, beta cells with mutation in GCK gene show less response to increments of ambient glucose concentration (FIG. 3A). Cells are also transplanted into the kidney capsule of immunocompromised mice (NSG). After 2-3 month, human c-peptide can be detected in the serum of the receipt mice. Cells from a human ES cell line and a control iPS cell line showed about 4 fold induction in c-peptide secretion after glucose injection. The beta cells carrying GCK mutation are clearly less responsive to increments in blood glucose concentration (FIG. 3B).

FIG. 4. GCK mutant stem cells are pluripotent Induced pluripotent stem cells (iPSCs) were generated from a patient with a missense mutation in GCK gene. The pluripotency of these iPS cells was verified by immunocytochemistry, embryoid body and teratoma formation assays. The resulting embryoid bodies and teratomas contained cell types of three germ layers-endoderm, mesoderm and ectoderm.

FIG. 5. Patient-specific stem cells give rise to beta-like cells. The patient-specific GCK mutant iPS cells were differentiated towards pancreatic endoderm and insulin-producing cells using a previously described stepwise approach (D'Amour et al., 2006; Maehr et al., 2009). Pancreatic progenitors can be efficiently generated (up to ˜80% PDX1 positive cells) and the resulting endocrine cells secreted hormones including insulin, glucagon and somatostatin.

FIG. 6. GCK mutant beta cells developed in vivo. Pancreatic endoderm mainly composed of PDX1 positive cells was transplanted into immunocompromised mice. These cells matured into insulin-producing cells that were able to secrete insulin and respond to increased glucose levels.

FIGS. 7A-E. An allelic series of glucokinase mutations in cells from a MODY2 subject. (FIG. 7A) Structure of the glucokinase gene (GCK) and cognate protein and nucleotide sequences of the mutations. Black boxes represent exons. (*) indicate the mutations (E256K and G299R). Green boxes represent ATP binding domains and yellow boxes represent substrate binding domains. (FIG. 7B) Schematic view of the first step of the gene correction procedure: exons 7 to 10 of GCK, either the mutant or the wild type allele, were replaced with a hygro-TK cassette. Sequences at the mutation site were analyzed by Sanger sequencing. P1 and P2 (blue arrows) were the primers used to detect integration of the hygro-TK. (FIG. 7C) Scheme of the second round of gene targeting replacing the hygro-TK cassette with the wild type locus marked by an intronic SNP (triangle). Both targeting steps were facilitated by site-specific endonucleases, a zinc-finger nuclease for the first step, and I-SceI for the second step. Green bars indicate the restriction sites, the red bar the probe used for Southern blot analysis. Blue bars represent primers used to screen and identify targeting events. PCR (with P1 and P3) and Sanger sequencing showed the corrected sequence at the mutation site and the intronic SNP that marks the corrected allele. (FIG. 7D) Southern blot analysis showing two bands representing the targeted allele (hygro, 1.5 kb) and the non-targeted allele (+ or G299R, 2.4 kb). (FIG. 7E) Karyotype analysis of GCK^{corrected/+} cells.

FIGS. 8A-H. Enhanced beta-like cell generation through calcium chelation and TGFβ signaling inhibition. (FIG. 8A) Morphology of control iPS cells after 1 day of Activin A treatment with and without EGTA. Boundaries of colonies are indicated by white lines. (FIGS. 8B-D) Differentiation of control iPS cells in the presence or absence of EGTA. Quantification of Oct4 positive Sox17 negative cells (FIG. 8B), Sox17 positive cells (FIG. 8C) after 3 days of differentiating control iPS cells, and quantification of pancreatic progenitor cells (PDX1+) (FIG. 8D) after 8 days of differentiation. ***: P<0.001. (FIG. 8E) Percentage of insulin-positive cells (stained for C-peptide) after treatments for 2 days with the indicated compounds (n=8 replicas). (All error bars in this figure represent Standard Error). (FIG. 8F) The mRNA expression of INS and GCK at definitive endoderm (DE), pancreatic endoderm (PE) and endocrine (EN) stages of differentiation, determined by semi-quantitative RT-PCR. TBP: TATA box binding protein. (FIG. 8G) Immunohistochemistry of explants isolated 4 months post transplantation. GCG: glucagon, SST: somatostatin, scale bar, 10 μm. (FIG. 8H) Measurement of human C-peptide levels in the mouse serum prior and after excision of the transplants. Shown were mice transplanted with GCK mutant cells (GCK^{G299R/+}). Error bars represent Standard Deviation.

FIGS. 9A-F. GCK gene dosage affects beta-like cell replication and glucose stimulated insulin secretion. (FIG. 9A) Insulin content of control cells, GCK mutant and gene-corrected cells. (FIG. 9B) Insulin (C-peptide) secretion in response to indicated secretagogues in vitro. (FIG. 9C) Glucose-stimulated insulin (C-peptide) secretion in control, GCK mutant and gene-corrected cells in vitro. (FIG. 9D) Glucose-stimulated insulin release into the mouse circulation from the transplanted cells (n>=3 animals). pES1 is a parthenogenetic ES cell line (31). (FIG. 9E) Differentiation efficiency of GCK G299R mutant and gene-corrected cells. (FIG. 9F) Proportion of in vitro differentiated beta-like cells that were Ki67-positive. (Error bars in B, C, D and E represent Standard Deviation. Error bars in F represent Standard Error, n=20 replicates).

FIG. 10. Pedigrees of the MODY2 subjects (marked in red).

FIGS. 11A-F. GCK mutant iPS cells are pluripotent. (FIG. 11A) Fibroblast cell line and induced pluripotent cells were derived from a MODY2 subject carrying a hypomorphic mutation (G299R) in the glucokinase gene (GCK). (FIG. 11B) iPS cells from the two MODY2 subjects had normal karyotypes. (FIG. 11C) A cluster tree showing global gene expression profiles of iPS cells and fibroblast cells of control and MODY2 subjects. (FIG. 11D) Pluripotency marker genes expressed in the stem cells generated from two MODY2 subjects. (FIG. 11E) Embryoid bodies formed by GCK mutant stem cells contained three germ layers-endoderm (AFP+), mesoderm (MF20+) and ectoderm (Tuj1+). (FIG. 11F) GCK mutant stem cells formed teratomas that contained tissue structures from three germ layers.

FIGS. 12A-F. Characterization of beta-like cells derived in vitro. (FIG. 12A) Efficiency of generating pancreatic progenitors and insulin-producing cells using a published protocol (1). * indicates that no insulin positive cells were detected. (FIG. 12B) Distribution of SOX17+ and OCT4+ cells after 3 days of differentiation following the published protocol. (FIG. 12C) Expression of endocrine hormones after 12 days of differentiation and diagrams showing proportion of insulin and glucagon (left) or insulin and somatostatin (right)-producing cells. CPEP: C-peptide, GCG: glucagon, SST: somatostatin. (FIG. 12D) Electron microscope images of insulin producing cells derived from ES cells and GCK^{G299R/+} cells. (FIG. 12E) Quantification by EM of insulin granule numbers per insulin-producing cell, by genotype. Not different by genotype. (n=3 per genotype). (FIG. 12F) Differentiation efficiency of GCK^{E256K/+} and control cells.

FIG. 13. Immunostaining of beta cells derived in vitro (day 14). Scale bar: 50 μm.

FIGS. 14A-D. Beta cells derived in vivo display characteristics of mature beta cells. (FIG. 14A) Human C-peptide concentrations in mouse serum collected at fasting state. (FIG. 14B) Measurement of human C-peptide levels in the mouse serum prior and after excision of the transplants. Shown were mice transplanted with GCK mutant cells (GCK^{G299R/+}). Error bars represent Standard Deviation. (FIG. 14C) Immunohistochemistry of explants isolated 4 months post transplantation of GCK mutant cells (GCK^{G299R/+}). INS: insulin, UCN-3: urocortin-3, ZNT8: zinc transporter 8. Scale bar, 100 μm. (FIG. 14D) Scatter plots showing fold change in c-PEP concentration (30 min after glucose injection versus 16 hours fasting) versus delta capillary blood glucose concentration (30 min after glucose injection minus 16 hours fasting) during IPGTT.

FIGS. 15A-B. GCK gene dosage specific affects glucose stimulated insulin secretion. (FIG. 15A) Fold change of glucose-stimulated insulin (C-peptide) secretion in human islets, control, GCK mutant and gene-corrected cells in vitro. The basal condition was 5.6 mM glucose and the stimulation condition was 16.9 mM glucose. Error bars represent standard deviation of 3 experiments. (FIG. 15B) Insulin (C-peptide) secretion in response to indicated secretagogues in vitro.

FIGS. 16A-B. Beta cells derived in vitro were not fully mature yet displayed insulin secretion defect specific to glucose. (FIG. 16A) Immunostaining of in vitro differentiated beta cells. INS: insulin, UCN-3: urocotin-3, ZNT8: zinc transporter 8. Scale bar, 100 μm. (FIG. 16B) Insulin (C-peptide) secretion of in vitro derived beta cells in response to glucose (20 mM) and potassium (30 mM). The basal condition was 2.5 mM glucose and 4.8 mM potassium. 5 out 8 control replicas showed response to glucose while none of the GCK mutant replicates did. All the control and GCK mutant replicates showed response to potassium.

FIGS. 17A-C. (FIG. 17A) Schematic illustration of HNF1A gene structure and the location and sequences of mutations present in the research subjects studied. (FIG. 17B) Representative images of immunostaining of in vitro differentiated beta cells derived from different individuals as indicated. INS: insulin. (FIG. 17C) Quantification graph showing the percentage of insulin positive cells derived from different individuals as indicated.

FIG. 18. Graph showing insulin mRNA levels of in vitro differentiated beta cells derived from different individuals as indicated, determined by RNA sequencing. FPKM: fragments per kilobase of exon per million fragments mapped.

FIG. 19. Graph showing the amount of C-peptide secreted per insulin positive cell in 1 hour (attomol/cell) of in vitro differentiated beta cells derived from different individuals as indicated.

FIGS. 20A-F. (FIG. 20A) Graph showing the fold-change of C-peptide secretion between 16.9 mM glucose challenge and 5.6 mM glucose of in vitro differentiated beta cells derived from individuals as indicated. (FIG. 20B) Graph showing the fold-change of C-peptide secretion between 15.3 mM arginine and 0.3 mM arginine of in vitro differentiated beta cells derived from different genetic backgrounds as indicated. (FIG. 20C) Graph showing the fold-change of C-peptide secretion between 30.5 mM KCl and 0.5 mM KCl of in vitro differentiated beta cells derived from different individuals as indicated. (FIG. 20D) Heat map showing expression of indicated genes in control and KD (HNF1A knock down) cells. Up-regulation (Pink) or down-regulation (Green) of genes indicated. (FIG. 20E) Correlation of gene expression for genes indicated between control and KD cells. (FIG. 20F) Graph showing relative mRNA levels of glucose transporter 1 (GLUT1), glucose transporter 2 (GLUT2) and glucokinase (GCK) in control, MODY and KD cells, determined by quantitative RT-PCR.

FIGS. 21A-B. (FIG. 21A) Graph showing insulin secreted within 1 hour (fmol) from cells co-cultured with indicated matrix for 1 or 5 weeks. The genotypes of in vitro differentiated beta cells are indicated. The materials of matrix are indicated: PXS, porcine pancreas; MG, matrigel; HRT, porcine heart. (FIG. 21B) Graph showing insulin secreted within 1 hour from cells co-cultured with porcine pancreas for 1 or 5 weeks. The genotypes of in vitro differentiated beta cells are indicated.

FIGS. 22A-D. (FIG. 22A) Graph showing immunostaining of Control or MODY insulin positive cells cultured in 5.6 mM glucose, 15.6 mM glucose or 0.2 mM palmitate for 5 days. (FIG. 22B) Graph showing fold change of insulin positive cell number in response to 15.6 mM glucose or 0.2 mM palmitate in in vitro differentiated beta cells derived from different individuals as indicated. (FIG. 22C) Fluorescence activated cell sorting to purify beta cells from human islets, control cells and MODY cells. (FIG. 22D) Graph showing the percentage of Ki67 positive cells in in vitro differentiated beta cells derived from different genetic backgrounds as indicated and cultured in 5.6 mM glucose, 15.6 mM glucose or 0.2 mM palmitate.

FIG. 23. Immunostaining for pluripotent marker genes Oct4, Tra1-60, Sox2 and Nanog in induced pluripotent stem cells derived from different individuals as indicated.

FIGS. 24A-E shows that induced pluripotent stem cells (iPSCs) from Wolfram subjects were efficiently differentiated into insulin-producing cells. FIG. 24A is a diagram of WFS1 structure showing the mutation sites and Sanger sequencing profiles in the 4 Wolfram subjects described herein. Arrows indicate the four deleted nucleotides (CTCT). FIG. 24B shows immunostaining of Wolfram cultures differentiated to endoderm (SOX17), pancreatic endoderm (PDX1) and C-peptide positive cells. FIG. 24C shows the differentiation efficiency in control and WFS1 cells using imaging. N=10 for each of 3 independent experiments. FIG. 24D is a representative FACS showing percentage of C-peptide positive cells in differentiated control and WFS1 cells. FIG. 24E shows immunostaining analysis of WFS1, glucagon and C-peptide in iPS-derived pancreatic Wolfram cell cultures.

FIGS. 25A-H shows that reduced insulin production in Wolfram beta cells can be rescued by ER stress reliever 4PBA. FIG. 25A shows insulin mRNA levels in control and WFS1 beta cells normalized to TBP mRNA levels and to the number of insulin positive cells used for analysis. FIG. 25B shows insulin protein content in control and WFS1 beta cells under indicated conditions. Error bars represents 3 independent experiments with three replicates in each experiment. FIG. 25C shows transmission electron microscope (TEM) images of representative control and WFS1 cells. Scale bar is 2 nm. FIG. 25D shows the quantification of granule numbers per section of control and WFS1 cells. Two independent experiments with n=9 sections for each subject of each experiment. FIG. 25E shows the fold change of spliced XBP-1 mRNA levels in control and Wolfram beta cell cultures treated with vehicle or 4PBA for 7 days. FIG. 25F shows the fold change of GRP78 mRNA level in control and Wolfram iPS cells at increasing concentration of TG treatment for 6 hours. * P<0.05. FIG. 25G shows the fold change of GRP78 mRNA levels in Wolfram iPSCs upon different treatments. * P<0.05. TG: thapsigargin; 10 nM. 4PBA: Sodium 4-phenylbutyrate; 1 mM. TUDCA: tauroursodeoxycholate; 1 mM. FIG. 25H shows representative TEM images showing endoplasmic reticulum morphology in control and WFS1 cells after 12 hours treatment of 10 nM TG. Arrows point to ER structure. Scale bar is 500 nm.

FIGS. 26A-D shows that insulin secretion function and insulin processing are more vulnerable to ER stress. FIG. 26A shows the fold change of human C-peptide secretion in response to indicated secretagogues. Cells were treated with 5.6 mM glucose for 1 hour followed by 16.9 mM glucose, or 15 mM arginine, or 30 mM potassium, or 1 mM DBcAMP+16.9 mM glucose. Results present three independent experiments with n=3 for each experiment. * P<0.05 of TG vs. Vehicle; #P<0.05 of TG+4PBA vs. TG. FIG. 26B shows the fold change of human C-peptide secretion to glucose stimulation calculated as amount of C-peptide secreted in response to 16.9 mM glucose divided by C-peptide secreted in response to 5.6 mM glucose. N=3 for each of two independent experiments. FIG. 26C shows the Proinsulin/insulin ratio in control and WFS1 cells under indicated conditions. N=6 for each of two independent experiments. FIG. 26D shows the fold change of human C-peptide and glucagon in control and WFS1 cells under indicated conditions. N=3 for each experiment of 3 independent experiments. TG: thapsigargin; 10 nM, 12 hour treatment. 4PBA: Sodium 4-phenylbutyrate; 1 mM, 1 hour treatment prior to and 12 hour during TG treatment.

FIGS. 27A-E shows that Wolfram beta cells showed reduced glucose response in vivo. FIG. 27A shows human C-peptide level in the sera of recipient and negative control mice before and after nephrectomy. FIG. 27B shows basal human C-peptide level in the sera of mice transplanted with human islets, control and WFS1 cells. FIG. 27C shows the fold change of human C-peptide in the sera of mice transplanted with human islets, control and WFS1 cells before and 30 mins after glucose (1 mg/g body weight) IP injection. FIG. 27D shows the fold change of human C-peptide levels (before and after glucose injection) produced by human islets and WFS1 implants during 90 day period. FIG. 27E shows immunohistochemistry analysis of transplanted control and WFS1 beta cells. Representative images showing human C-peptide and ATF6.alpha. positive cells in transplants.

FIGS. 28A-D shows that induced pluripotent stem (iPS) cells generated from Wolfram fibroblasts using Sendai virus vectors. FIG. 28A. Wolfram subject fibroblasts and Wolfram subject iPS cells. FIG. 28B. Karyotypes of the iPS cells of four Wolfram research subjects. FIG. 28C. The Wolfram iPS cells expressed pluripotent marker genes, shown are SSEA4, SOX2, TRA-1-60, NANOG, TRA-1-81, OCT4, by immunocytochemistry. FIG. 28D shows immunohistochemistry of embryonic body cultures and histological analysis of teratomas derived from iPS cells.

FIGS. 29A-C shows enhanced unfolded protein response in Wolfram cells. FIG. 29A. Basal GRP78 mRNA levels in Control and Wolfram iPS cells. Quantification represents the results from studies of 4 Wolfram subject lines of three independent experiments. FIG. 29B. Gel image showing splicing of XBP-1 mRNA level in control and Wolfram iPS cells under indicated conditions and quantification represents the results from studies of 4 Wolfram subject lines of three independent experiments. FIG. 29C. Western blot analysis showing GRP78 expression level in control and Wolfram fibroblasts under indicated conditions. Quantification represents the results from studies from 2 Wolfram subjects (WS-1 and WS-2) of three independent experiments. TM: tunicamycin; 4PBA: Sodium 4-phenylbutyrate.

FIGS. 30A-B shows insulin secretion of Wolfram beta cells derived from Wolfram iPSCs generated by using retrovirus vectors, instead of Sendai virus. FIG. 30A. Fold change of human C-peptide secretion to 16.9 mM glucose stimulation in control and Wolfram beta cells. N=3 for each experiment of three independent experiments. FIG. 30B. Expression from the retroviral transgenes in different cell lines as indicated. This shows that the viral vectors expression was silenced in the iPS cells.

FIG. 31 shows insulin secretion of Wolfram beta cells upon tunicamycin (TM) treatment. Fold change of human C-peptide secretion to 30 mM potassium stimulation in control and Wolfram beta cells. N=3 for each experiment of three independent experiments. 4PBA: Sodium 4-phenylbutyrate.

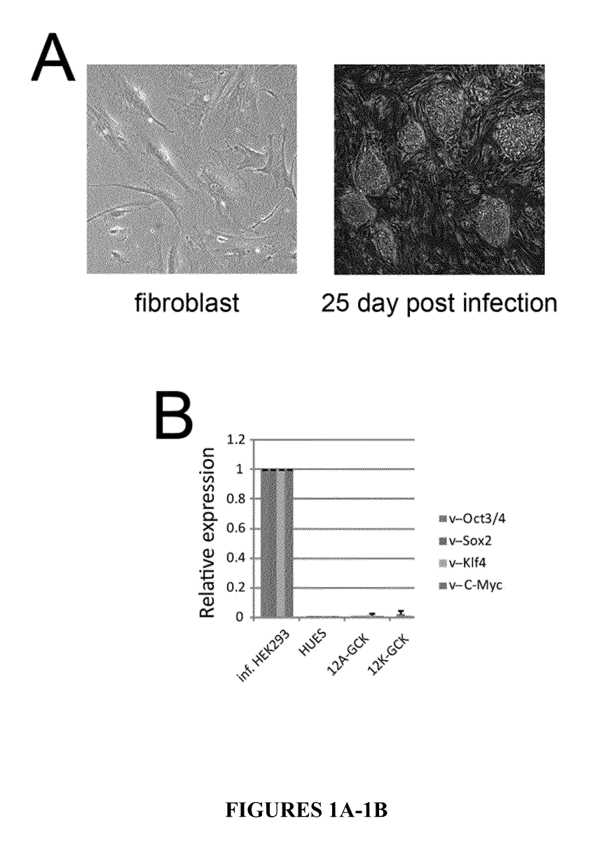

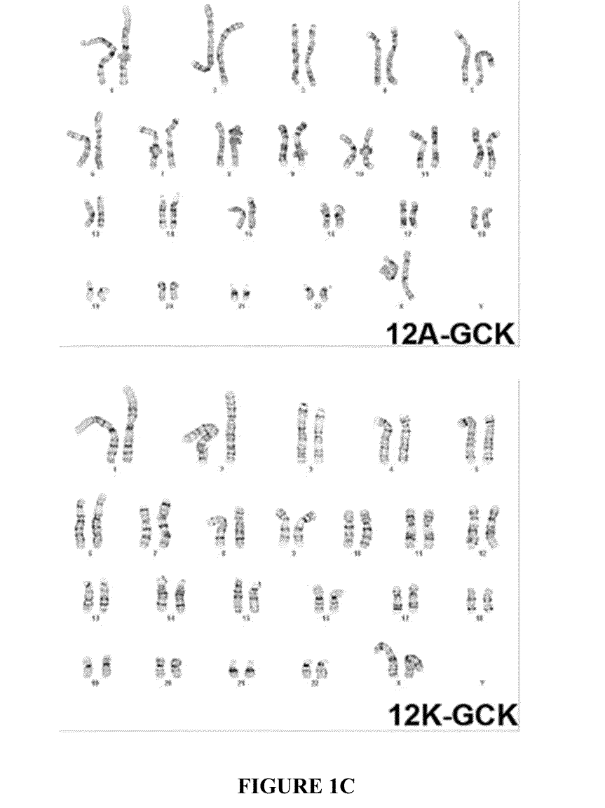

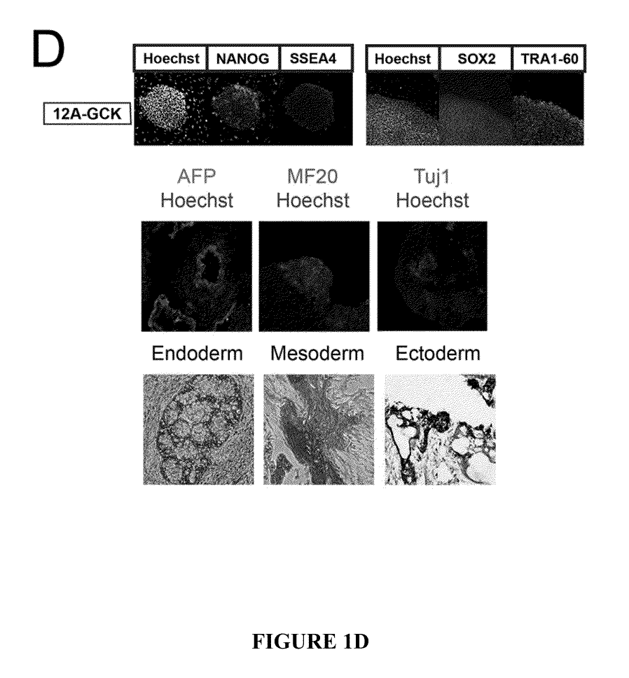

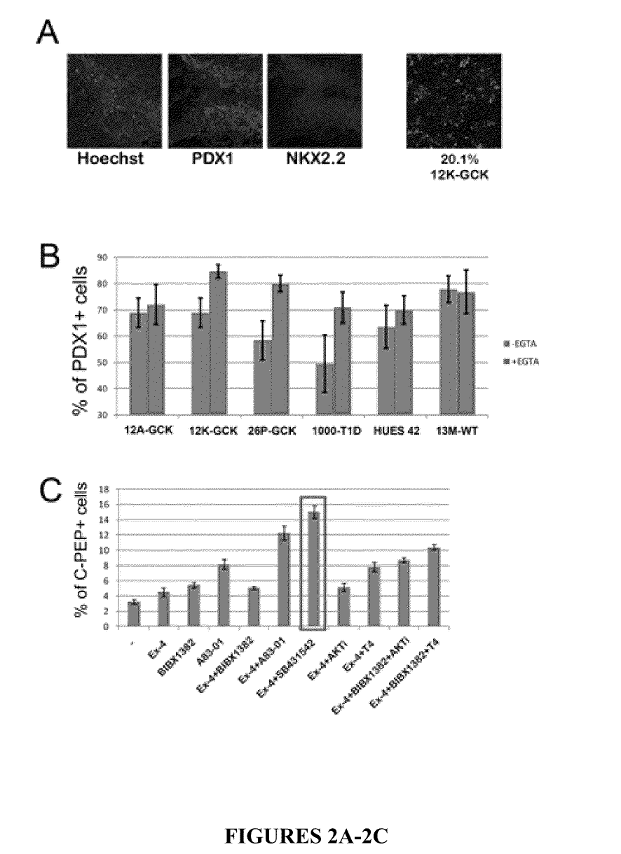

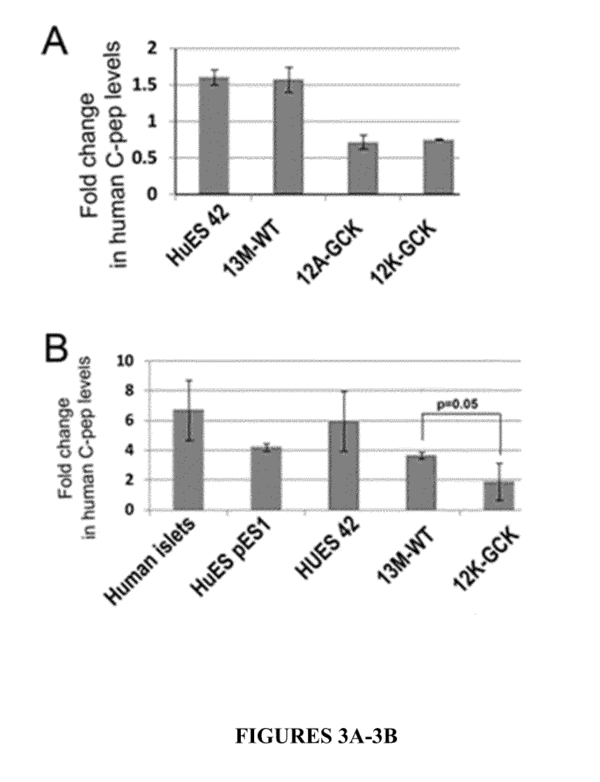

### DETAILED DESCRIPTION

The singular forms “a,” “an,” and “the” include plural reference unless the context clearly dictates otherwise.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Method for Generating Beta Cells

Provided is an in vitro method for generating pancreatic progenitor cells, insulin producing cells or endoderm cells using embryonic stem cells and induced pluripotent stem cells. The present invention also relates to an isolated population comprising pancreatic progenitor cells or a insulin-producing cells, compositions and their use in the treatment of diabetes.

The disclosure relates to methods comprising generation of induced pluripotent stem cells from mammal with mutations causing diabetes, efficient production of insulin-producing cells from embryonic stem cells and induced pluripotent stem cells, evaluate functionality of stem cell-derived insulin-producing cells and compositions thereof.

In certain embodiments, the pancreatic progenitor cells, insulin producing cells or endoderm cells described herein are can be obtained from a preparation of stem cells (e.g. human embryonic stem cells) or induced pluripotent stem cells that are undergoing or have undergone cell culture under standard procedures and conditions that are known in the art. In certain embodiments, prior to differentiation, the stem cells (e.g. human embryonic stem cells) or induced pluripotent stem cells are detached and dissociated using Dispase (3-5 min @ RT) and, subsequently, Accutase (3-5 min @ RT). The detached stem cells (e.g. human embryonic stem cells) or induced pluripotent stem cells are then suspended in human ES medium with ROCK inhibitor (Y27632) and filtered through 70 um (or 100 um) cell strainer. After filtration, the stem cells (e.g. human embryonic stem cells) or induced pluripotent stem cells are seeded a density of about 400,000 to about 800,000 cells/well (6-well plate) or about 200,000 to about 400,000 cell/well (12-well plate) or about 50,000 to about 200,000 cell/well (24-well plate) or about 25,000 to about 50,000 cell/well (96-well). The seeded stem cells (e.g. human embryonic stem cells) or induced pluripotent stem cells are then grown for about 24 hours to about 48 hours. In certain embodiments, the seeded stem cells (e.g. human embryonic stem cells) or induced pluripotent stem cells are grown until the culture reaches confluence.

After the about 24 hours to about 48 hours of growth, one Day 1 the seeded stem cells (e.g. human embryonic stem cells) or induced pluripotent stem cells are washed once with RPMI medium (with 1× Pen-Strep, 1× Glutamax). The seeded stem cells (e.g. human embryonic stem cells) or induced pluripotent stem cells are then cultured in RPMI medium (with 1× Pen-Strep, 1× Glutamax) containing human Activin A protein (about 100 ng/ml), human Wnt3A protein (about 25 ng/ml) and about 0.15 mM Ethylene glycol-bis(2-aminoethylether)-N,N,N′,N′-tetraacetic acid. On Day 2 and 3, the cells are then cultured in RPMI medium (with 1× Pen-Strep, 1× Glutamax) containing human Activin A protein (about 100 ng/ml) and about 0.2% FBS (by volume) in RPMI medium (with 1× Pen-Strep, 1× Glutamax). On Day 4 and 5 the cells are then cultured in RPMI medium (with 1× Pen-Strep, 1× Glutamax) containing human FGF10 protein (about 50 ng/ml), KAAD-cyclopamine (about 0.25 uM) and about 2% FBS. On Day 6, 7 and 8, the cells are cultured in DMEM (high glucose) medium (with 1× Pen-Strep, 1× Glutamax) containing human FGF10 protein (about 50 ng/ml), KAAD-cyclopamine (about 0.25 uM), retinoic acid (about 2 uM) and LDN-193189 (about 250 nM) and 1×B27. On Day 9 and 10, the cells are cultured in CMRL medium (with 1× Pen-Strep, 1× Glutamax) containing exedin-4 (about 50 ng/ml), SB431542 (about 2 uM) and 1×B27. On Day 11 and 12, cells are culture in CMRL medium (with 1× Pen-Strep, 1× Gutamax) containing 4-(4-Hydroxy-3,5-diiodophenoxy)-3,5-diiodobenzeneacetic acid and 1×B27. The resulting pancreatic progenitor cells, insulin producing cells or endoderm cells can be maintained in CMRL medium (with 1× Pen-Strep, 1× Glutamax) containing 1×B27.

The cell culture methods described herein can comprise culturing on an impermeable substrate, a permeable substrate, a transwell substrate, in suspension in liquid media, or by embedding in a 2D or 3D gel or matrix. Exemplary matrices include suitable for use with the methods described herein include, but are not limited to, Matrigel, collagen gel, laminin gel, as well as artificial 3D lattices constructed from materials such as polylactic acid or polyglycolic acid.

In certain aspect, the methods for generating pancreatic progenitor cells, insulin producing cells or endoderm cells from a preparation of stem cells (e.g. human embryonic stem cells) or induced pluripotent stem cell comprise steps of, (a) contacting the cells to a first culture medium, wherein the first culture medium is an RPMI medium (with 1× Pen-Strep, 1× Glutamax) comprising human Activin A protein, human Wnt3A protein and Ethylene glycol-bis(2-aminoethylether)-N,N,N′,N′-tetraacetic acid, (b) contacting the cells to a second culture medium, wherein the second culture medium is an RPMI medium (with 1× Pen-Strep, 1× Glutamax) containing human Activin A protein and FBS in RPMI medium (with 1× Pen-Strep, 1× Glutamax), (c) contacting the cells to a third culture medium, wherein the third culture medium is an RPMI medium (with 1× Pen-Strep, 1× Glutamax) containing human FGF10 protein, KAAD-cyclopamine and FBS in RPMI medium (with 1× Pen-Strep, 1× Glutamax), (d) contacting the cells to a fourth culture medium, wherein the fourth culture medium is an DMEM high glucose medium (with 1× Pen-Strep, 1× Glutamax) containing human FGF10 protein, KAAD-cyclopamine, retinoic acid, LDN-193189 and 1×B27, (e) contacting the cells to a fifth culture medium, wherein the fifth culture medium is a CMRL medium (with 1× Pen-Strep, 1× Glutamax) containing exedin-4, SB431542 and 1×B27, (f) contacting the cells to a sixth culture medium, wherein the sixth culture medium is a CMRL medium (with 1× Pen-Strep, 1× Glutamax) containing 4-(4-Hydroxy-3,5-diiodophenoxy)-3,5-diiodobenzeneacetic acid and 1×B27.

In certain embodiments, the concentration of human Activin A protein in the first culture RPMI medium can be about 100 ng/ml. In certain embodiments, the concentration of human Wnt3A protein in the first culture RPMI medium can be about 25 ng/ml. In certain embodiments, the concentration of Ethylene glycol-bis(2-aminoethylether)-N,N,N′,N′-tetraacetic acid in the first culture RPMI medium can be about 0.15 mM. In certain embodiments, the cells are cultured in the first culture RPMI medium for a period of about 24 hours. In certain embodiments, the first culture RPMI medium does not comprise an antibiotic. In certain embodiments, the first culture RPMI medium comprises EGTA.

In certain embodiments, the concentration of human Activin A protein in the second culture RPMI medium can be about 100 ng/ml. In certain embodiments, the concentration of FBS in the second culture RPMI medium can be about 0.2% FBS (by volume) in RPMI medium (with 1× Pen-Strep, 1× Glutamax). In certain embodiments, the cells are cultured in the second culture RPMI medium for a period of about 48 hours. In certain embodiments, the second culture RPMI medium is replaced with fresh second culture RPMI medium about 24 hours after the cells are first exposed to the second culture RPMI medium. In certain embodiments, the second culture RPMI medium does not comprise an antibiotic. In certain embodiments, the second culture RPMI medium comprises EGTA.

In certain embodiments, the concentration of human FGF10 protein in the third culture RPMI medium can be about 50 ng/ml. In certain embodiments, the concentration of KAAD-cyclopamine in the third culture RPMI medium can be about 0.25 uM. In certain embodiments, the concentration of FBS in the third culture RPMI medium can be about 2% FBS in RPMI medium (with 1× Pen-Strep, 1× Glutamax). In certain embodiments, the cells are cultured in the third culture RPMI medium for a period of about 48 hours. In certain embodiments, the third culture RPMI medium is replaced with fresh third culture RPMI medium about 24 hours after the cells are first exposed to the third culture RPMI medium. In certain embodiments, the third culture RPMI medium does not comprise an antibiotic. In certain embodiments, the third culture RPMI medium comprises EGTA.

In other embodiments, the third culture medium is modified, wherein the third culture medium is an RPMI medium (with 1× Pen-Strep, 1× Glutamax) containing human KGF protein and FBS in RPMI medium (with 1× Pen-Strep, 1× Glutamax). In one embodiment, the concentration of human KGF in the third culture RPMI medium can be about 50 ng/ml. In certain embodiments, the concentration of FBS in the third culture RPMI medium can be about 2% FBS in RPMI medium (with 1× Pen-Strep, 1× Glutamax). In certain embodiments, the cells are cultured in the third culture RPMI medium for a period of about 48 hours. In certain embodiments, the third culture RPMI medium is replaced with fresh third culture RPMI medium about 24 hours after the cells are first exposed to the third culture RPMI medium. In certain embodiments, the third culture RPMI medium does not comprise an antibiotic. In certain embodiments, the third culture RPMI medium comprises EGTA.

In certain embodiments, the concentration of human FGF10 protein in the fourth culture DMEM high glucose medium can be about 50 ng/ml. In certain embodiments, the concentration of KAAD-cyclopamine in the fourth culture DMEM high glucose medium can be about 0.25 uM. In certain embodiments, the concentration of retinoic acid in the fourth culture DMEM high glucose medium can be about 2 uM. In certain embodiments, the concentration of LDN-193189 in the fourth culture DMEM high glucose medium can be about 250 nM. In certain embodiments, the cells are cultured in the fourth culture DMEM high glucose medium for a period of about 72 hours. In certain embodiments, the fourth culture DMEM high glucose medium is replaced with fresh fourth culture DMEM high glucose medium about 24 hours after the cells are first exposed to the fourth culture DMEM high glucose medium. In certain embodiments, the fourth culture DMEM high glucose medium is replaced with fresh fourth culture DMEM high glucose medium about 48 hours after the cells are first exposed to the fourth culture DMEM high glucose medium. In certain embodiments, the fourth culture DMEM high glucose medium is replaced with fresh fourth culture DMEM high glucose medium about 24 hours after the cells are first exposed to the fourth culture DMEM high glucose medium. In certain embodiments, the fourth culture DMEM high glucose medium is replaced with fresh fourth culture DMEM high glucose medium about 24 hours and about 48 hours after the cells are first exposed to the fourth culture DMEM high glucose medium. In certain embodiments, the fourth culture DMEM high glucose medium does not comprise an antibiotic. In certain embodiments, the fourth culture DMEM high glucose medium comprises EGTA.

In other embodiments, the fourth culture medium is modified, wherein the fourth culture medium is an DMEM high glucose medium (with 1× Pen-Strep, 1× Glutamax) containing KAAD-cyclopamine, 4-[(E)-2-(5,6,7,8-Tetrahydro-5,5,8,8-tetramethyl-2-naphthalenyl)-1-propenyl]benzoic acid (TTNPB), LDN-193189, Activin A and 1×B27. In certain embodiments, the concentration of KAAD-cyclopamine in the fourth culture DMEM high glucose medium can be about 0.25 uM. In certain embodiments, the concentration of 4-[(E)-2-(5,6,7,8-Tetrahydro-5,5,8,8-tetramethyl-2-naphthalenyl)-1-propenyl]benzoic acid (TTNPB) can be about 3 nM. In certain embodiments, the concentration of LDN-193189 in the fourth culture DMEM high glucose medium can be about 250 nM. In certain embodiments, the concentration of Activin A can be about 100 ng/ml. In certain embodiments, the cells are cultured in the fourth culture DMEM high glucose medium for a period of about 72 hours. In certain embodiments, the fourth culture DMEM high glucose medium is replaced with fresh fourth culture DMEM high glucose medium about 24 hours after the cells are first exposed to the fourth culture DMEM high glucose medium. In certain embodiments, the fourth culture DMEM high glucose medium is replaced with fresh fourth culture DMEM high glucose medium about 48 hours after the cells are first exposed to the fourth culture DMEM high glucose medium. In certain embodiments, the fourth culture DMEM high glucose medium is replaced with fresh fourth culture DMEM high glucose medium about 24 hours after the cells are first exposed to the fourth culture DMEM high glucose medium. In certain embodiments, the fourth culture DMEM high glucose medium is replaced with fresh fourth culture DMEM high glucose medium about 24 hours and about 48 hours after the cells are first exposed to the fourth culture DMEM high glucose medium. In certain embodiments, the fourth culture DMEM high glucose medium does not comprise an antibiotic. In certain embodiments, the fourth culture DMEM high glucose medium comprises EGTA.

In certain embodiments, the concentration of exedin-4 in the fifth culture CMRL medium can be about 50 ng/ml. In certain embodiments, the concentration of SB431542 in the fifth culture CMRL medium can be about 2 uM. In certain embodiments, the cells are cultured in the fifth culture CMRL medium for a period of about 48 hours. In certain embodiments, the fifth culture CMRL medium is replaced with fresh fifth culture CMRL medium about 24 hours after the cells are first exposed to the fifth culture CMRL medium. In certain embodiments, the fifth culture CMRL medium does not comprise an antibiotic. In certain embodiments, the fifth culture CMRL medium comprises EGTA.

In other embodiments, the fifth culture medium is modified, wherein the fifth culture medium is a DMEN high glucose medium (with 1× Pen-Strep, 1× Glutamax) containing exedin-4, ALK5 inhibitor and 1×B27. In certain embodiments, the concentration of exedin-4 in the fifth culture DMEM high glucose medium can be about 50 ng/ml. In certain embodiments, the concentration of ALK5 inhibitor in the fifth culture DMEM high glucose medium can be about 1 uM. In certain embodiments, the cells are cultured in the fifth culture DMEM high glucose medium for a period of about 48 hours. In certain embodiments, the fifth culture DMEM high glucose medium is replaced with fresh fifth culture DMEM high glucose medium about 24 hours after the cells are first exposed to the fifth culture DMEM high glucose medium. In certain embodiments, the fifth culture DMEM high glucose medium does not comprise an antibiotic. In certain embodiments, the fifth culture DMEM high glucose medium comprises EGTA.

In certain embodiments, the concentration of 4-(4-Hydroxy-3,5-diiodophenoxy)-3,5-diiodobenzeneacetic acid (about 20 pM) in the sixth culture CMRL medium can be about 20 pM. In certain embodiments, the cells are cultured in the sixth culture CMRL medium for a period of about 48 hours. In certain embodiments, the sixth culture CMRL medium is replaced with fresh sixth culture CMRL medium about 24 hours after the cells are first exposed to the sixth culture CMRL medium. In certain embodiments, the sixth culture CMRL medium does not comprise an antibiotic. In certain embodiments, the sixth culture CMRL medium comprises EGTA.

In certain embodiments, the pancreatic progenitor cells, insulin producing cells or endoderm cells generated according to the methods described herein can be maintained in CMRL medium (with 1× Pen-Strep, 1× Glutamax) containing 1×B27. In certain embodiments, the pancreatic progenitor cells, insulin producing cells or endoderm cells generated according to the methods described herein can be maintained in CMRL medium (with 1× Glutamax) containing 1×B27, without any antibiotic. In certain embodiments, the CMRL medium used to maintain the pancreatic progenitor cells, insulin producing cells or endoderm cells generated according to the methods described herein can further comprise EGTA.

In certain aspects, the methods described herein relates to a method for producing a pancreatic progenitor cell from a human embryonic stem cell or from an induced pluripotent stem cell. In certain aspects, the methods described herein relates to a method for producing an insulin producing cell from a human embryonic stem cell or from an induced pluripotent stem cell. In certain aspects, the methods described herein relates to a method for producing an endoderm cell from a human embryonic stem cell or from an induced pluripotent stem cell.

In certain embodiments, the pancreatic progenitor cells, insulin producing cells or endoderm cells generated according to the methods described herein express transcription factors, including but not limited to, PDX-1 and NKX6.1.

In certain embodiments, the methods described herein comprise steps of contacting a human embryonic stem cell or an induced pluripotent stem cell with a combination of various factors, including, but not limited to EGTA. In certain embodiments, the methods described herein relate to the finding that the use of EGTA in connection with the methods described herein improves the efficiency of generating pancreatic progenitor cells, insulin producing cells or endoderm cells from a human embryonic stem cell of from an induced pluripotent cell.

In certain embodiments, the methods described herein comprise steps of contacting a human embryonic stem cell or an induced pluripotent stem cell with a combination of various factors, including, but not limited to exendin-4. In certain embodiments, the methods described herein relate to the finding that the use of exendin-4 in connection with the methods described herein improves the efficiency of generating pancreatic progenitor cells, insulin producing cells or endoderm cells from a human embryonic stem cell of from an induced pluripotent cell.

In certain embodiments, the methods described herein comprise steps of contacting a human embryonic stem cell or an induced pluripotent stem cell with a combination of various factors, including, but not limited to SB431542. In certain embodiments, the methods described herein relate to the finding that the use of SB431542 in connection with the methods described herein improves the efficiency of generating pancreatic progenitor cells, insulin producing cells or endoderm cells from a human embryonic stem cell of from an induced pluripotent cell.

In certain embodiments, the induced pluripotent stem cells suitable for use with the methods described herein are from a human. In certain aspects, the methods described herein allow for the generation of pancreatic progenitor cells, insulin producing cell or endoderm without using embryos, oocytes, and/or nuclear transfer technology. In certain embodiments, the induced pluripotent stem cells suitable for use with the methods described herein comprise a mutation causing diabetes. In certain embodiments, the induced pluripotent stem cells suitable for use with the methods described herein can be a cell from a mammal (e.g. a mouse or a human) having Type I diabetes, Type II diabetes and/or pre-diabetes, or a mammal (e.g. a mouse or a human) at risk of having Type I diabetes, Type II diabetes and/or pre-diabetes.

In certain aspect, the methods for generating pancreatic progenitor cells, insulin producing cells or endoderm cells from a preparation of stem cells (e.g. human embryonic stem cells) or induced pluripotent stem cell comprise steps of, (a) contacting the cells to a first culture medium, wherein the first culture medium is an RPMI medium (with 1× Pen-Strep, 1× Glutamax) comprising human Activin A protein, human Wnt3A protein and Ethylene glycol-bis(2-aminoethylether)-N,N,N′,N′-tetraacetic acid, (b) contacting the cells to a second culture medium, wherein the second culture medium is an RPMI medium (with 1× Pen-Strep, 1× Glutamax) containing human Activin A protein and FBS in RPMI medium (with 1× Pen-Strep, 1× Glutamax), (c) contacting the cells to a third culture medium, wherein the third culture medium is an RPMI medium (with 1× Pen-Strep, 1× Glutamax) containing human KGF protein and FBS in RPMI medium (with 1× Pen-Strep, 1× Glutamax), (d) contacting the cells to a fourth culture medium, wherein the fourth culture medium is an DMEM high glucose medium (with 1× Pen-Strep, 1× Glutamax) containing KAAD-cyclopamine, 4-[(E)-2-(5,6,7,8-Tetrahydro-5,5,8,8-tetramethyl-2-naphthalenyl)-1-propenyl]benzoic acid (TTNPB), LDN-193189, Activin A and 1×B27, (e) contacting the cells to a fifth culture medium, wherein the fifth culture medium is a DMEN high glucose medium (with 1× Pen-Strep, 1× Glutamax) containing exedin-4, ALK5 inhibitor and 1×B27, (f) contacting the cells to a sixth culture medium, wherein the sixth culture medium is a CMRL medium (with 1× Pen-Strep, 1× Glutamax) containing 4-(4-Hydroxy-3,5-diiodophenoxy)-3,5-diiodobenzeneacetic acid and 1×B27.

In certain embodiments, the concentration of human Activin A protein in the first culture RPMI medium can be about 100 ng/ml. In certain embodiments, the concentration of human Wnt3A protein in the first culture RPMI medium can be about 25 ng/ml. In certain embodiments, the concentration of Ethylene glycol-bis(2-aminoethylether)-N,N,N′,N′-tetraacetic acid in the first culture RPMI medium can be about 0.15 mM. In certain embodiments, the cells are cultured in the first culture RPMI medium for a period of about 24 hours. In certain embodiments, the first culture RPMI medium does not comprise an antibiotic. In certain embodiments, the first culture RPMI medium comprises EGTA.

In certain embodiments, the concentration of human Activin A protein in the second culture RPMI medium can be about 100 ng/ml. In certain embodiments, the concentration of FBS in the second culture RPMI medium can be about 0.2% FBS (by volume) in RPMI medium (with 1× Pen-Strep, 1× Glutamax). In certain embodiments, the cells are cultured in the second culture RPMI medium for a period of about 48 hours. In certain embodiments, the second culture RPMI medium is replaced with fresh second culture RPMI medium about 24 hours after the cells are first exposed to the second culture RPMI medium. In certain embodiments, the second culture RPMI medium does not comprise an antibiotic. In certain embodiments, the second culture RPMI medium comprises EGTA.

In certain embodiments, the concentration of human KGF in the third culture RPMI medium can be about 50 ng/ml. In certain embodiments, the concentration of FBS in the third culture RPMI medium can be about 2% FBS in RPMI medium (with 1× Pen-Strep, 1× Glutamax). In certain embodiments, the cells are cultured in the third culture RPMI medium for a period of about 48 hours. In certain embodiments, the third culture RPMI medium is replaced with fresh third culture RPMI medium about 24 hours after the cells are first exposed to the third culture RPMI medium. In certain embodiments, the third culture RPMI medium does not comprise an antibiotic. In certain embodiments, the third culture RPMI medium comprises EGTA.

In certain embodiments, the concentration of KAAD-cyclopamine in the fourth culture DMEM high glucose medium can be about 0.25 uM. In certain embodiments, the concentration of 4-[(E)-2-(5,6,7,8-Tetrahydro-5,5,8,8-tetramethyl-2-naphthalenyl)-1-propenyl]benzoic acid (TTNPB) can be about 3 nM. In certain embodiments, the concentration of LDN-193189 in the fourth culture DMEM high glucose medium can be about 250 nM. In certain embodiments, the concentration of Activin A can be about 100 ng/ml. In certain embodiments, the cells are cultured in the fourth culture DMEM high glucose medium for a period of about 72 hours. In certain embodiments, the fourth culture DMEM high glucose medium is replaced with fresh fourth culture DMEM high glucose medium about 24 hours after the cells are first exposed to the fourth culture DMEM high glucose medium. In certain embodiments, the fourth culture DMEM high glucose medium is replaced with fresh fourth culture DMEM high glucose medium about 48 hours after the cells are first exposed to the fourth culture DMEM high glucose medium. In certain embodiments, the fourth culture DMEM high glucose medium is replaced with fresh fourth culture DMEM high glucose medium about 24 hours after the cells are first exposed to the fourth culture DMEM high glucose medium. In certain embodiments, the fourth culture DMEM high glucose medium is replaced with fresh fourth culture DMEM high glucose medium about 24 hours and about 48 hours after the cells are first exposed to the fourth culture DMEM high glucose medium. In certain embodiments, the fourth culture DMEM high glucose medium does not comprise an antibiotic. In certain embodiments, the fourth culture DMEM high glucose medium comprises EGTA.

In certain embodiments, the concentration of exedin-4 in the fifth culture DMEM high glucose medium can be about 50 ng/ml. In certain embodiments, the concentration of ALK5 inhibitor in the fifth culture DMEM high glucose medium can be about 1 uM. In certain embodiments, the cells are cultured in the fifth culture DMEM high glucose medium for a period of about 48 hours. In certain embodiments, the fifth culture DMEM high glucose medium is replaced with fresh fifth culture DMEM high glucose medium about 24 hours after the cells are first exposed to the fifth culture DMEM high glucose medium. In certain embodiments, the fifth culture DMEM high glucose medium does not comprise an antibiotic. In certain embodiments, the fifth culture DMEM high glucose medium comprises EGTA.

In certain embodiments, the concentration of 4-(4-Hydroxy-3,5-diiodophenoxy)-3,5-diiodobenzeneacetic acid (about 20 pM) in the sixth culture CMRL medium can be about 20 pM. In certain embodiments, the cells are cultured in the sixth culture CMRL medium for a period of about 48 hours. In certain embodiments, the sixth culture CMRL medium is replaced with fresh sixth culture CMRL medium about 24 hours after the cells are first exposed to the sixth culture CMRL medium. In certain embodiments, the sixth culture CMRL medium does not comprise an antibiotic. In certain embodiments, the sixth culture CMRL medium comprises EGTA.

In certain embodiments, the pancreatic progenitor cells, insulin producing cells or endoderm cells generated according to the methods described herein can be maintained in CMRL medium (with 1× Pen-Strep, 1× Glutamax) containing 1×B27. In certain embodiments, the pancreatic progenitor cells, insulin producing cells or endoderm cells generated according to the methods described herein can be maintained in CMRL medium (with 1× Glutamax) containing 1×B27, without any antibiotic. In certain embodiments, the CMRL medium used to maintain the pancreatic progenitor cells, insulin producing cells or endoderm cells generated according to the methods described herein can further comprise EGTA.

In certain embodiments, the methods described herein comprise steps of contacting a human embryonic stem cell or an induced pluripotent stem cell with a combination of various factors, including, but not limited to human KGF. In certain embodiments, the methods described herein relate to the finding that the use of human KGF in connection with the methods described herein improves the efficiency of generating pancreatic progenitor cells, insulin producing cells or endoderm cells from a human embryonic stem cell of from an induced pluripotent cell.

In certain embodiments, the methods described herein comprise steps of contacting a human embryonic stem cell or an induced pluripotent stem cell with a combination of various factors, including, but not limited to 4-[(E)-2-(5,6,7,8-Tetrahydro-5,5,8,8-tetramethyl-2-naphthalenyl)-1-propenyl]benzoic acid (TTNPB). In certain embodiments, the methods described herein relate to the finding that the use of 4-[(E)-2-(5,6,7,8-Tetrahydro-5,5,8,8-tetramethyl-2-naphthalenyl)-1-propenyl]benzoic acid (TTNPB) in connection with the methods described herein improves the efficiency of generating pancreatic progenitor cells, insulin producing cells or endoderm cells from a human embryonic stem cell of from an induced pluripotent cell.

In certain embodiments, the methods described herein comprise steps of contacting a human embryonic stem cell or an induced pluripotent stem cell with a combination of various factors, including, but not limited to ALK5 inhibitor. In certain embodiments, the methods described herein relate to the finding that the use of ALK5 inhibitor in connection with the methods described herein improves the efficiency of generating pancreatic progenitor cells, insulin producing cells or endoderm cells from a human embryonic stem cell of from an induced pluripotent cell.

In certain embodiments, prior to differentiation, the stem cells (e.g. human embryonic stem cells) or induced pluripotent stem cells are detached and dissociated using TrypLE (Invitrogen). The detached stem cells (e.g. human embryonic stem cells) or induced pluripotent stem cells are then suspended in human ES medium with ROCK inhibitor (Y27632) and filtered through 70 um (or 100 um) cell strainer. After filtration, the stem cells (e.g. human embryonic stem cells) or induced pluripotent stem cells are seeded at a density of about 400,000 to about 800,000 cells/well (12-well plate) or about 800,000 to about 1,000,000 cell/well (6-well plate) or about 50,000 to about 200,000 cell/well (24-well plate) or about 25,000 to about 50,000 cell/well (96-well). The seeded stem cells (e.g. human embryonic stem cells) or induced pluripotent stem cells are then grown for about 24 hours to about 48 hours. In certain embodiments, the seeded stem cells (e.g. human embryonic stem cells) or induced pluripotent stem cells are grown until the culture reaches confluence.

After the about 24 hours to about 72 hours of growth, on Day 1 the seeded stem cells (e.g. human embryonic stem cells) or induced pluripotent stem cells are cultured into definitive endoderm using STEMdiff Definitive Endoderm Kit Media (STEMCELL Technologies). On Day 4 and 5 the cells are then cultured in RPMI medium (with 1× Pen-Strep, 1× Glutamax) containing about 2% FBS (by volume) and KGF (about 50 ng/ml). On Day 6, 7 and 8 the cells are cultured in DMEM (high glucose) medium (with 1× Pen-Strep) containing KAAD-cyclopamine (about 0.25 uM), retinoic acid (about 2 uM) and LDN-193189 (about 250 nM) and 1×B27. On Day 9, 10, 11 and 12, the cells are cultured in DMEN (high glucose) medium (1× Pen-Strep) containing exendin-4 (about 50 ng/ml), ALK5 inhibitor II (about 1 uM) and 1×B27. On Day 13, cells are culture in CMRL medium (with 1× Pen-Strep, 1× Gutamax) containing 1×B27. The resulting pancreatic progenitor cells, insulin producing cells or endoderm cells can be maintained in CMRL medium (with 1× Pen-Strep, 1× Glutamax) containing 1×B27.

In certain aspect, the methods for generating pancreatic progenitor cells, insulin producing cells or endoderm cells from a preparation of stem cells (e.g. human embryonic stem cells) or induced pluripotent stem cell comprise steps of, (a) contacting the cells to a first culture medium, wherein the first culture medium is STEMdiff Definitive Endoderm Kit Media, (b) contacting the cells to a second culture medium, wherein the second culture medium is an RPMI medium (with 1× Pen-Strep, 1× Glutamax) containing FBS and human KGF in RPMI medium (with 1× Pen-Strep, 1× Glutamax), (c) contacting the cells to a third culture medium, wherein the third culture medium is a DMEM high glucose medium (with 1× Pen-Strep) containing KAAD-cyclopamine, retinoic acid and LDN-193189 and 1×B27, (d) contacting the cells to a fourth culture medium, wherein the fourth culture medium is an DMEM high glucose medium (with 1× Pen-Strep) containing exendin-4, ALK5 inhibitor II and 1×B27, (e) contacting the cells to a fifth culture medium, wherein the fifth culture medium is a CMRL medium (with 1× Pen-Strep, 1× Glutamax) containing 1×B27.

In certain embodiments, the concentration of human KGF in the second culture RPMI medium can be about 50 ng/ml. In certain embodiments, the concentration of FBS in the second culture RPMI medium can be about 2% FBS (by volume) in RPMI medium (with 1× Pen-Strep, 1× Glutamax). In certain embodiments, the cells are cultured in the second culture RPMI medium for a period of about 24 hours. In certain embodiments, the cells are cultures in the second culture medium for a period of about 48 hours. In certain embodiments, the second culture RPMI medium does not comprise an antibiotic. In certain embodiments, the second culture RPMI medium comprises EGTA.

In certain embodiments, the concentration of KAAD-cyclopamine in the third culture DMEM high glucose medium can be about 0.25 uM. In certain embodiments, the concentration of retinoic acid in the third culture DMEM high glucose medium can be about 2 uM. In certain embodiments, the concentration of LDN-193189 in the third culture DMEM high glucose medium can be about 250 nM. In certain embodiments, the cells are cultured in the third culture DMEM high glucose medium for a period of about 48 hours. In certain embodiments, the third culture DMEM high glucose medium is replaced with fresh third culture DMEM high glucose medium about 24 hours after the cells are first exposed to the third culture DMEM high glucose medium. In certain embodiments, the third culture DMEM high glucose medium is replaced with fresh third culture DMEM high glucose medium about 48 hours after the cells are first exposed to the third culture DMEM high glucose medium. In certain embodiments, the third culture DMEM high glucose medium is replaced with fresh third culture DMEM high glucose medium about 24 hours after the cells are first exposed to the third culture DMEM high glucose medium. In certain embodiments, the third culture DMEM high glucose medium is replaced with fresh third culture DMEM high glucose medium about 24 hours and about 48 hours after the cells are first exposed to the third culture DMEM high glucose medium. In certain embodiments, the third culture DMEM high glucose medium does not comprise an antibiotic. In certain embodiments, the third culture DMEM high glucose medium comprises EGTA.

In certain embodiments, the concentration of exendin-4 in the fourth culture DMEM high glucose medium can be about 50 ng/ml. In certain embodiments, the concentration of ALK5 inhibitor II in the fourth culture DMEM high glucose medium can be about 1 uM. In certain embodiments, the cells are cultured in the fourth culture DMEM high glucose medium for a period of about 72 hours. In certain embodiments, the fourth culture DMEM high glucose medium is replaced with fresh fourth culture DMEM high glucose medium about 24 hours after the cells are first exposed to the fourth culture DMEM high glucose medium. In certain embodiments, the fourth culture DMEM high glucose medium is replaced with fresh fourth culture DMEM high glucose medium about 48 hours after the cells are first exposed to the fourth culture DMEM high glucose medium. In certain embodiments, the fourth culture DMEM high glucose medium is replaced with fresh fourth culture DMEM high glucose medium about 24 hours after the cells are first exposed to the fourth culture DMEM high glucose medium. In certain embodiments, the fourth culture DMEM high glucose medium is replaced with fresh fourth culture DMEM high glucose medium about 24 hours and about 48 hours after the cells are first exposed to the fourth culture DMEM high glucose medium. In certain embodiments, the fourth culture DMEM high glucose medium does not comprise an antibiotic. In certain embodiments, the fourth culture DMEM high glucose medium comprises EGTA.

In certain embodiments, the pancreatic progenitor cells, insulin producing cells or endoderm cells generated according to the methods described herein can be maintained in CMRL medium (with 1× Pen-Strep, 1× Glutamax) containing 1×B27. In certain embodiments, the pancreatic progenitor cells, insulin producing cells or endoderm cells generated according to the methods described herein can be maintained in CMRL medium (with 1× Glutamax) containing 1×B27, without any antibiotic. In certain embodiments, the CMRL medium used to maintain the pancreatic progenitor cells, insulin producing cells or endoderm cells generated according to the methods described herein can further comprise EGTA.

In certain embodiments, the methods described herein comprise steps of contacting a human embryonic stem cell or an induced pluripotent stem cell with a combination of various factors, including, but not limited to human KGF. In certain embodiments, the methods described herein relate to the finding that the use of human KGF in connection with the methods described herein improves the efficiency of generating pancreatic progenitor cells, insulin producing cells or endoderm cells from a human embryonic stem cell of from an induced pluripotent cell.

In certain embodiments, the methods described herein comprise steps of contacting a human embryonic stem cell or an induced pluripotent stem cell with a combination of various factors, including, but not limited to ALK5 inhibitor II. In certain embodiments, the methods described herein relate to the finding that the use of ALK5 inhibitor II in connection with the methods described herein improves the efficiency of generating pancreatic progenitor cells, insulin producing cells or endoderm cells from a human embryonic stem cell of from an induced pluripotent cell.

As used herein, the term diabetes refers to a syndrome that can be characterized by disordered metabolism resulting in abnormally high blood sugar levels (hyperglycemia). The two most common forms of diabetes are due to either a diminished production of insulin (in Type 1), or diminished response by the body to insulin (in Type 2 and gestational). Type 1 diabetes (Type 1 diabetes, Type I diabetes, T1D, T1DM, IDDM, juvenile diabetes) is a disease that results in the permanent destruction of insulin-producing beta cells of the pancreas. Type 2 diabetes (non-insulin-dependent diabetes mellitus (NIDDM), or adult-onset diabetes) is a metabolic disorder that is primarily characterized by insulin resistance (diminished response by the body to insulin), relative insulin deficiency, and hyperglycemia. Complications associated with diabetes include, but are not limited to hypoglycemia, ketoacidosis, or nonketotic hyperosmolar coma, cardiovascular disease, renal failure, retinal damage, nerve damage, and microvascular damage. In some embodiments, a mammal is pre-diabetic, which can be characterized, for example, as having elevated fasting blood sugar or elevated post-prandial blood sugar.

In certain embodiments, the induced a pluripotent stem cell suitable for use with the methods described herein is a cell that does not comprise a mutation causing diabetes.

The induced pluripotent stem cells suitable for use with the methods described herein can also be cells derived from tissue formed after gestation, including pre-embryonic tissue (e.g. a blastocysts), embryonic tissue, or fetal tissue taken during gestation (e.g. after about 10-12 weeks of gestation). Non-limiting examples of induced pluripotent stem cells suitable for use with the methods described herein include established lines of human embryonic stem cells or human embryonic germ cells, such as, for example the human embryonic stem cell lines H1, H7, and H9 (WiCell). In certain embodiments, induced pluripotent stem cells suitable for use with the methods described herein include cells generated according to the methods described in Thomson et al. (U.S. Pat. No. 5,843,780; Science 282:1145, 1998; Curr. Top. Dev. Biol. 38:133 ff., 1998; Proc. Natl. Acad. Sci. U.S.A. 92:7844, 1995). Also suitable for use with the methods described herein include, but are not limited to, induced pluripotent stem cells taken directly from a source tissue, cells from a induced pluripotent stem cell population cultured in the absence of feeder cells such as, pluripotent stem cells that are supported using a medium conditioned by culturing previously with another cell type.

In certain embodiments, the induced pluripotent stem cells suitable for use with the methods described herein include pluripotent stem cells cultured on a layer of feeder cells that support the proliferation of the pluripotent stem cells without causing the pluripotent stem cells to undergo substantial differentiation. Methods for proliferating pluripotent stem cells suitable for use with the methods described herein include, but are not limited, to those disclosed in Reubinoff et al (Nature Biotechnology 18: 399-404 (2000)); Thompson et al (Science 6 Nov. 1998: Vol. 282. no. 5391, pp. 1145-1147); Richards et al, (Stem Cells 21: 546-556, 2003); US20020072117; Wang et al (Stem Cells 23: 1221-1227, 2005); Stojkovic et al (Stem Cells 2005 23: 306-314, 2005); Miyamoto et al (Stem Cells 22: 433-440, 2004); Amit et al (Biol. Reprod 68: 2150-2156, 2003); Inzunza et al (Stem Cells 23: 544-549, 2005); U.S. Pat. No. 6,642,048; WO2005014799; Xu et al (Stem Cells 22: 972-980, 2004); or US20070010011.

Other induced pluripotent stem cells suitable for use with the method described herein include, but are not limited to those obtained, grown or proliferated according to the methods set forth in Cheon et al (BioReprod DOI:10.1095/biolreprod.105.046870, Oct. 19, 2005); Levenstein et al (Stem Cells 24: 568-574, 2006); US20050148070; US20050233446; U.S. Pat. No. 6,800,480; US20050244962; WO2005065354; or WO2005086845. Pluripotent stem cells suitable for use with the methods described herein also include pluripotent stem cells grown in a cell culture substrate comprising an extracellular matrix component (e.g. laminin, fibronectin, proteoglycan, entactin).

The induced pluripotent cells described herein can be obtained according to any method known in the art. In certain embodiments the induced pluripotent cells suitable for use with the methods described herein can be obtained by dedifferentiating or reprogramming a source cell.

In certain embodiments, a source cell suitable for obtaining the induced pluripotent stem cells suitable for use with the methods described herein can be a fibroblast. For example, in certain embodiments the induced pluripotent cells suitable for use with the methods described herein can be obtained according to a method comprising the steps of (a) obtaining a source cell by taking a skin biopsy from a mammal (e.g. a mouse or a human), (b) establishing a fibroblast cell line from the skin biopsy, (c) infecting the fibroblast cell line with a retrovirus or a sendai virus capable of directing expression of human transcription factors Oct4, Sox2, Klf4 and C-Myc in the fibroblast cell line. In certain embodiments, one or more colonies of induced pluripotent stem cells can be isolated 3 weeks after infection with the retrovirus or sendai virus. In certain embodiments, the isolated one or more colonies of induced pluripotent stem cells can be expanded to establish one or more induced pluripotent stem cells.

In certain embodiments, induced pluripotent stem cells are derived from fibroblasts using CytoTune™-iPS Sendai Reprogramming Kit (Invitrogen). In one embodiment, fibroblast cells are seeded at a density of about 50,000/well (6-well plate). The seeded fibroblasts are then grown for about 24 hours to about 48 hours. In one embodiment, the seeded fibroblasts are between about passages 2 and 5. The seeded fibroblasts are then infected with a retrovirus or a Sendai virus capable of directing expression of human transcription factors Oct4, Sox2, Klf4 and C-Myc. In certain embodiments, after infection with the retrovirus or Sendai virus the cells are cultured and about 3 to 4 weeks later colonies of induced pluripotent stem cells can be isolated. In certain embodiments, the isolated one or more colonies of induced pluripotent stem cells can be expanded to establish one or more induced pluripotent stem cells.

In certain embodiments, a source cell suitable for obtaining the induced pluripotent stem cells suitable for use with the methods described herein can be a cell of endoderm origin. In certain embodiments, the cell of endoderm origin suitable for use as a source cell can be a non-insulin producing cell from a population of pancreatic cells, including but not limited to an exocrine cell, a pancreatic duct cell, and acinar pancreatic cell. In certain embodiments, the cell of endoderm origin suitable for use as a source cell can be a non-insulin producing cell from a population of liver cells or a population of gall bladder cells. Another aspect of the present invention relates to a method for the treatment of a mammal (e.g. a mouse or a human) with diabetes, the method comprising administering a composition comprising the pancreatic progenitor cells, insulin producing cells or endoderm cells generated according to the methods described herein. Another aspect of the present invention relates to the use of the pancreatic progenitor cells, insulin producing cell or endoderm produced by the methods as disclosed herein for administering to a mammal in need thereof. In some embodiments, the pancreatic progenitor cells, insulin producing cell or endoderm are produced from stem cells, induced stem cells or source cells from the same mammal as to whom the composition is administered. In some embodiments, the mammal has, or has an increased risk of developing, diabetes, for example, where the mammal has, or has increased risk of getting diabetes from the group consisting of: Type I diabetes, Type II diabetes and pre-diabetes. In certain embodiments, the pancreatic progenitor cells, insulin producing cells or endoderm cells generated according to the methods described herein secrete at least about 5%, at least about 15%, at least about 25%, at least about 35%, at least about 45%, at least about 55%, at least about 65%, at least about 75%, at least about 85%, at least about 95%, or more than about 100% of the amount of insulin secreted by an endogenous beta cell in the presence of a stimulating agent.

In certain aspects, the invention relates to methods for characterizing the pancreatic progenitor cells, insulin producing cell or endoderm generated according to the methods described herein. In certain embodiments, the pancreatic progenitor cells, insulin producing cell or endoderm generated according to the methods described herein can be characterized by measuring insulin secretion in response to stimuli. Stimuli suitable for inducing insulin secretion include, but are not limited to, glucose and potassium. For example, the pancreatic progenitor cells, insulin producing cell or endoderm generated according to the methods described herein can be characterized by washing them in CMRL medium comprising about 5.6 mM glucose for about one hour. Then cell can then be incubated in CMRL medium comprising about 5.6 mM glucose for about one hour and the medium can then be collected. The cells can then be incubated in CMRL medium comprising about 16.9 mM glucose or about 35 mM potassium for about one hour and the medium can be collected. The levels of human c-peptide in the media can then be measured as indicator of insulin secretion. In certain embodiments, insulin secretion of the pancreatic progenitor cells, insulin producing cell or endoderm generated according to the methods described herein can be compared to insulin secretion by an endogenous beta-cell from a mammal (e.g. a human). In certain embodiments, the pancreatic progenitor cells, insulin producing cells or endoderm cells generated according to the methods described herein secrete at least about 5%, at least about 15%, at least about 25%, at least about 35%, at least about 45%, at least about 55%, at least about 65%, at least about 75%, at least about 85%, at least about 95%, or more than about 100% of the amount of insulin secreted by an endogenous beta cell in the absence of a stimulating agent.

In certain aspect, the invention relates to transplantation of the pancreatic progenitor cells, insulin producing cells or endoderm cells generated according to the methods described herein. In certain embodiments, the pancreatic progenitor cells, insulin producing cells or endoderm cells generated according to the methods described herein can be transplanted into non-human mammal (e.g. a mouse). In certain embodiments, the pancreatic progenitor cells, insulin producing cells or endoderm cells generated according to the methods described herein can be transplanted into a human. In one embodiment, transplantation of the pancreatic progenitor cells, insulin producing cells or endoderm cells generated according to the methods described herein into a non-human mammal or a human can be performed by trypsin digestion and suspension in CMRL medium for 12-24 hours. The cells can then be collected and transplanted under a kidney capsule. For example, transplantation in a non-human mammal can be under the kidney capsule of one NSG mouse.

In certain embodiments, the pancreatic progenitor cells, insulin producing cells or endoderm cells produced according to the methods described herein can be used for the production of a pharmaceutical composition, for the use in transplantation into mammals in need of treatment, e.g. a mammal that has, or is at risk of developing diabetes, for example but not limited to mammal with congenital and acquired diabetes. In certain embodiments, an isolated population of the pancreatic progenitor cells, insulin producing cells or endoderm cells produced according to the methods described herein may be genetically modified.

The use of an isolated population of the pancreatic progenitor cells, insulin producing cells or endoderm cells produced according to the methods described herein provides advantages over existing methods because the pancreatic progenitor cells, insulin producing cells or endoderm cells produced according to the methods described herein can be generated from cells obtained from a mammal in need of therapeutic intervention or from another mammal of the same species.

In certain embodiments, the invention relates to a method of treating diabetes or a metabolic disorder in a mammal comprising administering an effective amount of a composition comprising a population of pancreatic progenitor cells, insulin producing cells or endoderm cells produced according to the methods described herein to a mammal with diabetes or pre-diabetes. In a further embodiment, the invention provides a method for treating diabetes, comprising administering a composition comprising a population of pancreatic progenitor cells, insulin producing cells or endoderm cells produced according to the methods described herein to a mammal that has, or has increased risk of developing diabetes in an effective amount sufficient to produce insulin in response to increased blood glucose levels.

In certain embodiments, the mammal is a human and a population of pancreatic progenitor cells, insulin producing cells or endoderm cells produced according to the methods described herein are human cells. In some embodiments, a population of pancreatic progenitor cells, insulin producing cells or endoderm cells produced according to the methods described herein can be administered to any suitable location in the mammal, for example in a capsule in the blood vessel or the liver or any suitable site where administered population of pancreatic progenitor cells, insulin producing cells or endoderm cells produced according to the methods described herein can secrete insulin in response to increased glucose levels in the mammal. In some embodiments, a population of pancreatic progenitor cells, insulin producing cells or endoderm cells produced according to the methods described herein can be introduced by injection, catheter, or the like.

In some embodiments, a population of pancreatic progenitor cells, insulin producing cells or endoderm cells produced according to the methods described herein can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition comprising a population of pancreatic progenitor cells, insulin producing cells or endoderm cells produced according to the methods described herein will be adapted in accordance with the route and device used for administration. In some embodiments, a composition comprising a population of pancreatic progenitor cells, insulin producing cells or endoderm cells produced according to the methods described herein can also comprise or be accompanied with one or more other ingredients that facilitate the engraftment or functional mobilization of the pancreatic progenitor cells, insulin producing cells or endoderm cells produced according to the methods described herein. Suitable ingredients include matrix proteins that support or promote adhesion of the pancreatic progenitor cells, insulin producing cells or endoderm cells produced according to the methods described herein. In another embodiment, the composition may comprise resorbable or biodegradable matrix scaffolds.

In certain aspects, a population of pancreatic progenitor cells, insulin producing cells or endoderm cells produced according to the methods described herein can be for administered systemically or to a target anatomical site. A population of pancreatic progenitor cells, insulin producing cells or endoderm cells produced according to the methods described herein can be grafted into or nearby a mammal's pancreas, for example, or may be administered systemically, such as, but not limited to, intra-arterial or intravenous administration. In alternative embodiments, a population of pancreatic progenitor cells, insulin producing cells or endoderm cells produced according to the methods described herein invention can be administered in various ways as would be appropriate to implant in the pancreatic or secretory system, including but not limited to parenteral, including intravenous and intraarterial administration, intrathecal administration, intraventricular administration, intraparenchymal, intracranial, intracisternal, intrastriatal, and intranigral administration. A population of pancreatic progenitor cells, insulin producing cells or endoderm cells produced according to the methods described herein can also be administered in conjunction with an immunosuppressive agent.

In certain embodiments, a population of pancreatic of pancreatic progenitor cells, insulin producing cells or endoderm cells produced according to the methods described herein can be administered and dosed in accordance with good medical practice, taking into account the clinical condition of the individual patient, the site and method of administration, scheduling of administration, patient age, sex, body weight and other factors known to medical practitioners. A pharmaceutically “effective amount” for purposes herein is thus determined by such considerations as are known in the art. The amount can be effective to achieve improvement, including but not limited to improved survival rate or more rapid recovery, or improvement or elimination of symptoms and other indicators as are selected as appropriate measures by those skilled in the art.

In some embodiments, a population of pancreatic progenitor cells, insulin producing cells or endoderm cells produced according to the methods described herein may be administered in any physiologically acceptable excipient, where the cells may find an appropriate site for regeneration and differentiation.

Another aspect of the present invention further provides a method of treating diabetes in a mammal diagnosed with Type 1 diabetes, comprising administering to the mammal a population of pancreatic progenitor cells, insulin producing cells or endoderm cells produced according to the methods described herein. In certain embodiments the treatment methods described herein can be combined with other methods of treating Type I diabetes, Type II diabetes or pre-diabetes, including but not limited to lowering blood glucose in a mammal, inhibiting gluconeogenesis in a mammal, decreasing post-prandial glucose in a mammal or administering an anti-diabetic agent to the mammal Examples of anti-diabetic agents suitable for use with administration of the pancreatic progenitor cells, insulin producing cells or endoderm cells produced according to the methods described herein include a glucosidase inhibitor, a thiazolidinedione (e.g., TZD), an insulin sensitizer, a glucagon-like peptide-1 (GLP-1), insulin, a PPAR alpha/gamma dual agonist, an aP2 inhibitor and/or a DPP4 inhibitor. Examples of a glucosidase inhibitor include acarbose (disclosed in U.S. Pat. No. 4,904,769), voglibose, miglitol (disclosed in U.S. Pat. No. 4,639,436), which may be administered in a separate dosage form or the same dosage form. Examples of a PPAR gamma agonist includes a thiazolidinedione (e.g., TZD) such as rosiglitazone, pioglitazone, englitazone, and darglitazone.

In certain embodiments, a population of pancreatic progenitor cells, insulin producing cells or endoderm cells produced according to the methods described herein can be applied alone or in combination with other cells, tissue, tissue fragments, growth factors such as VEGF and other known angiogenic or arteriogenic growth factors, biologically active or inert compounds, resorbable plastic scaffolds, or other additive intended to enhance the delivery, efficacy, tolerability, or function of the population.

In certain embodiments, the methods described herein can also be used to treat a mammal having a diabetic condition which occurs as a consequence of genetic defect, physical injury, environmental insult or conditioning, bad health, obesity and other diabetes risk factors commonly known by a person of ordinary skill in the art.

In certain embodiments, a population of pancreatic progenitor cells, insulin producing cells or endoderm cells produced according to the methods described herein is stored for later implantation/infusion. In some embodiments, a population of pancreatic progenitor cells, insulin producing cells or endoderm cells produced according to the methods described herein can be frozen at liquid nitrogen temperatures and stored for long periods of time, being capable of use on thawing. If frozen, a population of pancreatic progenitor cells, insulin producing cells or endoderm cells produced according to the methods described herein can be stored in medium (e.g. a CMRL medium (with 1× Glutamax) containing 1×B27) comprising 10% DMSO. Once thawed, the cells may be expanded by use of growth factors and/or feeder cells suitable for culturing pancreatic progenitor cells, insulin producing cells or endoderm cells produced according to the methods described herein. Moderate to long-term storage of all or part of the cells in a cell bank is also within the scope of this invention, as disclosed in U.S. Patent Application Serial No. 20030054331 and Patent Application No. WO03024215, and is incorporated by reference in their entireties.

In certain aspects, the invention relates to a method for determining the functionality of the pancreatic progenitor cells, insulin producing cells or endoderm cells produced according to the methods described herein after they have been transplanted into a mammal (e.g. a mouse). In one embodiment, the functionality of such cells can be determined by measuring insulin secretion in response to glucose. In one embodiment, the mammal (e.g. the mouse) can be deprived of food for a period of about 12 hours to about 24 hours but will still have water available. The mammal can be weighed after the period of food deprivation and administered a glucose solution. In one embodiment, the administration of the glucose solution is by intraperitoneal injection. In one embodiment, the glucose solution is in saline and the amount of glucose injected is about 1 mg/g of body weight of the mammal. The mammal is then deprived of food or water. This can be accomplished by placing the mammal in an enclosure (e.g. a cage) lacking food or water. The blood glucose level of the mammal can then be examined at periodic intervals (e.g. every half hour). Blood samples can be collected before glucose injection and after half an hour of glucose injection. c-peptide levels in the blood serum can also be measured. Any method for measuring blood glucose or c-peptide level can be used in conjunction with the methods described herein. For example, for mice, blood can be obtained by tail vein bleeding. In certain embodiments, urine glucose concentrations can also be examined at periodic intervals.

In certain aspects, the pancreatic progenitor cells, insulin producing cells or endoderm cells produced according to the methods described herein can also be used to examine causal factors of beta cell phenotypes in diabetes. The methods for examining causal factors of beta cell phenotypes in diabetes can comprise evaluating functionality of pancreatic progenitor cells, insulin producing cells or endoderm cells produced according to the methods described herein both in-vitro (e.g. in cell culture) or upon transplantation into a mammal (e.g. a mouse). In certain embodiments, the pancreatic progenitor cells, insulin producing cells or endoderm cells produced according to the methods described herein can be genetically modified to examine whether one or more genes have a function in beta cell development and/or functionality show defects in insulin secretion in response to circulating glucose concentrations. In certain embodiments, the pancreatic progenitor cells, insulin producing cells or endoderm cells produced according to the methods described herein can generated from genetically modified stem cells or induced pluripotent cells to examine whether one or more genes have a function in beta cell development and/or functionality show defects in insulin secretion in response to circulating glucose concentrations.

In certain aspects, the pancreatic progenitor cells, insulin producing cells or endoderm cells produced according to the methods described herein can be used to screen test agents (e.g. compounds in a small molecule library) to identify agents capable of attenuating phenotypes arising from genetic defects that cause beta cell dysfunction.

In certain aspects, the pancreatic progenitor cells, insulin producing cells or endoderm cells produced according to the methods described herein can be used to screen test agents (e.g. compounds in a small molecule library) to identify agents capable of enhancing the efficiency of generating insulin-producing cells from stem cells or induced pluripotent cells.

In some embodiments, a population of pancreatic progenitor cells, insulin producing cells or endoderm cells produced according to the methods described herein may be genetically altered in order to introduce genes useful in the cells, e.g. repair of a genetic defect in an individual, selectable marker. In some embodiments, a population of pancreatic progenitor cells, insulin producing cells or endoderm cells produced according to the methods described herein can also be genetically modified to enhance survival, control proliferation, and the like. In some embodiments, a population of pancreatic progenitor cells, insulin producing cells or endoderm cells produced according to the methods described herein can be genetically altering by transfection or transduction with a suitable vector, homologous recombination, or other appropriate technique, so that they express a gene of interest. In one embodiment, a pancreatic progenitor cells, insulin producing cells or endoderm cells produced according to the methods described herein is transfected with genes encoding a telomerase catalytic component (TERT), typically under a heterologous promoter that increases telomerase expression beyond what occurs under the endogenous promoter, (see International Patent Application WO 98/14592, which is incorporated herein by reference). In other embodiments, a selectable marker is introduced, to provide for greater purity of the population of pancreatic progenitor cells, insulin producing cells or endoderm cells produced according to the methods described herein.

In certain aspects, the pancreatic progenitor cells, insulin producing cells or endoderm cells produced according to the methods described herein can be modified to express one or more exogenous nucleic acid sequences or genetically modified to alter expression of an endogenous nucleic acid sequence or genetically altered to reduce or eliminate expression of an endogenous nucleic acid sequence.

Genetic modification of the pancreatic progenitor cells, insulin producing cells or endoderm cells produced according to the methods described herein can be by insertion of DNA or by placement in cell culture in such a way as to change, enhance, or supplement the function of the cells for derivation of a structural or therapeutic purpose. For example, gene transfer techniques for stem cells are known by persons of ordinary skill in the art, as disclosed in (Morizono et al., 2003; Mosca et al., 2000), and may include gene gun technology, liposome-mediated transduction, and viral transfection techniques.

In certain embodiments, genetic alteration of the pancreatic progenitor cells, insulin producing cells or endoderm cells produced according to the methods described herein can be accomplished with a vector capable of directing expression of a nucleic acid sequence. Directed expression of the nucleic acid sequence can be driven from a promoter operatively linked to the nucleic acid sequence. The promoter can be constitutive or directed regulated expression (e.g. in a tissue specific, temporally regulated or inducible manner). Suitable inducible promoters include, but are not limited to, those that can be drive expression of a nucleic acid in a desired target cell type, either the transfected cell, or progeny thereof.

Many vectors useful for transferring exogenous nucleic acid into target pancreatic progenitor cells, insulin producing cells or endoderm cells produced according to the methods described herein are known in the art. The vectors may be episomal, e.g. plasmids, virus derived vectors such as cytomegalovirus, adenovirus, etc., or may be integrated into the target cell genome, through homologous recombination or random integration, e.g. retrovirus derived vectors such MMLV, HIV-1, ALV, etc. Commonly used retroviral vectors are “defective”, i.e. unable to produce viral proteins required for productive infection.

ER Stress Relievers in Beta Cell Protection

All forms of diabetes are ultimately caused by an inability of beta cells in the pancreas to provide sufficient insulin in response to ambient blood glucose concentrations. Autoimmunity in Type 1 diabetes (T1D) and peripheral insulin resistance in Type 2 diabetes (T2D) are important initiating mechanisms, but may not be the only factors resulting in reductions of beta cell functionality and mass. In T1D, autoimmunity precedes diabetes for several years, and beta cells are still present more than 8 years after diagnosis, but these residual beta cells are functionally compromised. During development of T2D, beta cells may initially compensate for peripheral insulin resistance by increasing insulin production and beta cell mass, but eventually fail in both; at advanced stages, beta cell mass and functionality is greatly reduced. Diabetes can also be caused by mutations in genes involved in beta cell function, causing maturity onset diabetes of the young (MODY), such as mutations in GCK (glucokinase), KCNJ11 (a potassium channel), or WFS1 (Wolfram syndrome).

Diabetes mellitus is a serious metabolic disease that is defined by the presence of chemically elevated levels of blood glucose (hyperglycemia). The term diabetes mellitus encompasses several different hyperglycemic states. These states include Type 1 (insulin-dependent diabetes mellitus or IDDM) and Type 2 (non-insulin dependent diabetes mellitus or NIDDM) diabetes. The hyperglycemia present in individuals with Type 1 diabetes is associated with deficient, reduced, or nonexistent levels of insulin that are insufficient to maintain blood glucose levels within the physiological range. Conventionally, Type 1 diabetes is treated by administration of replacement doses of insulin, generally by a parenteral route.

Type 2 diabetes is an increasingly prevalent disease of aging. It is initially characterized by decreased sensitivity to insulin and a compensatory elevation in circulating insulin concentrations, the latter of which is required to maintain normal blood glucose levels.

Wolfram syndrome is characterized by juvenile-onset diabetes, optic atrophy, deafness and neurological degeneration. The disease is fatal and no treatments for the diabetes other than provision of exogenous insulin are available. Wolfram syndrome is caused by mutations in WFS1 gene, which is highly expressed in human islets. Postmortem analysis of pancreata of Wolfram subjects showed a selective loss of pancreatic beta cells. In the mouse, loss of the WFS1 gene results in impaired glucose-stimulated insulin secretion, upregulation of ER stress markers, reduced insulin content, and a selective loss of beta cells in pancreatic islets. How dysfunctional WFS1 causes these phenotypes is not clear. WFS1 deficiency was reported to reduce insulin processing and acidification in insulin granules of mouse beta cells, where low pH is necessary for insulin processing and granule exocytosis. In cultured human cells, ectopically expressed WFS1 localizes to the endoplasmic reticulum (ER), where it physically interacts with calmodulin in a Ca2+-dependent manner and modulates free Ca2+ homeostasis, which is crucial for protein folding and insulin exocytosis. WFS1-deficient mouse islets showed reduced glucose-stimulated rise in the cytosolic calcium. In mouse islets, following stimulation with high concentrations of glucose, WFS1 can also be found on the plasma membrane, where it interacts with adenylyl cyclase and stimulates cAMP synthesis, thereby promoting insulin secretion. In addition, WFS1 deficiency leads to the activation of the unfolded protein response (UPR) components, such as GRP78 (Bip) and XBP-1 and decreases the ubiquitination of ATF6.alpha. The unfolded protein response coordinates protein-folding capacity with transcriptional regulation and protein synthesis to mitigate ER stress. The UPR may be particularly important for beta cells, which have obligate high levels of protein production and secretion. Failure to resolve unfolded protein response results in persistent decreases in translation and a loss of cellular functionality, or in cell death by apoptosis.

The endoplasmic reticulum (ER) is a cellular compartment responsible for multiple important cellular functions including the biosynthesis and folding of newly synthesized proteins destined for secretion, such as insulin. A myriad of pathological and physiological factors perturb ER function and cause dysregulation of ER homeostasis, leading to ER stress. ER stress elicits a signaling cascade to mitigate stress, the unfolded protein response (UPR). As long as the UPR can relieve stress, cells can produce the proper amount of proteins and maintain ER homeostasis. If the UPR, however, fails to maintain ER homeostasis, cells will undergo apoptosis. Activation of the UPR is critical to the survival of insulin-producing pancreatic beta-cells with high secretory protein production. Any disruption of ER homeostasis in beta-cells can lead to cell death and contribute to the pathogenesis of diabetes.

In one embodiment, the present invention is based on the seminal discovery that certain small molecules can relieve ER stress, leading to increased insulin production in beta cells and improved insulin secretion. While not wanting to be bound by a particular theory, it is believed that the present invention methods may lead to increased beta cell survival as well. Using a cellular model of diabetes based on patient-derived induced pluripotent stem cells (iPSCs), it was found that beta cells derived from WFS1 mutant stem cells showed insulin processing and insulin secretion in response to various secretagogues comparable to healthy controls, but had lower total insulin content and increased activity of unfolded protein response (UPR) pathways. Importantly, the chemical chaperone 4-phenylbutyric Acid (PBA) reduced the activity of UPR pathways, and restored normal insulin content. In contrast, experimental ER stress further reduced insulin content, impaired insulin processing and abolished stimulated insulin secretion in Wolfram beta cells, while cells from controls remained unaffected. PBA protected beta cells from these detrimental effects of ER stress. These results show that ER stress plays a central role in beta cell dysfunction, and demonstrate that beta cell function can be improved using chemical chaperones.

In one embodiment, the invention provides a method of treating a disease or disorder in a subject, wherein the disease or disorder is characterized by intracellular endoplasmic reticulum (ER) stress, comprising administering to the subject, an effective amount of a compound that is an ER stress reliever, thereby treating the disease or disorder. In one aspect, the compound is 4-phenylbutyric acid (PBA) or Tauroursodeoxycholic acid (TUDCA). In a further aspect, the disease or disorder is diabetes (type 1 or type 2), Wolcott-Rallison syndrome, Permanent neonatal Diabetes, PERK−/− (global elevation or ER stress) or Wolfram syndrome.

In yet another embodiment, the invention provides a method of inhibiting beta cell loss in a subject with diabetes (type 1 or type 2), comprising administering to the subject, an effective amount of an ER stress reliever compound, thereby inhibiting beta cell loss in the subject. In one aspect, the compound is a small molecule. In certain aspects, the compound is 4-phenylbutyric Acid (PBA) or Tauroursodeoxychlic Acid (TUDCA).

In another aspect, the invention methods include further administering exogenous insulin to the subject. The subject can be any mammal, preferably a human.

In another embodiment, the invention provides a method of identifying a compound that is an ER stress reliever comprising contacting a beta cell, in vitro or in vivo, with a test compound and measuring the level of insulin produced or protein folding prior to and following contacting with the test compound, wherein an increase in insulin levels or alteration in protein folding after contacting is indicative of an ER stress reliever compound. In one aspect, the beta cell is derived from a subject having diabetes. The beta cells can be derived from a pluripotent stem cells of a subject with diabetes. Such pluripotent stem cells can be obtained by a number of methods such as the illustrative method shown herein, which is by iPSC. Other methods are well known in the art.

The present invention is based on the discovery that certain compounds are effective for improving the survival of beta cells in the pancreas. Based on the findings herein, the invention provides methods for treating diabetes and other diseases where survival of beta cells is important.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The terms “beta cell” or “pancreatic beta cell” are interchangeable as used herein and refer to cells in the pancreatic islets that are of the lineage of cells that produce insulin in response to glucose. Beta cells are found in the islets of Langerhans in the pancreas. Beta cells secrete insulin in a regulated fashion in response to blood glucose levels. In Type I or insulin dependent diabetes mellitus (IDDM) beta cells are destroyed through an auto-immune process. Since the body can no longer produce endogenous insulin, injections of exogenous insulin are required to maintain normal blood glucose levels.

As used herein, the term “treatment,” when used in the context of a therapeutic strategy to treat a disease or disorder, means any manner in which one or more of the symptoms of a disease or disorder are ameliorated or otherwise beneficially altered. As used herein, amelioration of the symptoms of a particular disease or disorder refers to any lessening, whether permanent or temporary, lasting or transient that can be attributed to or associated with treatment by the compositions and methods of the present invention (e.g., promotion of beta cell survival; increased insulin production in a subject).

The terms “effective amount” and “effective to treat,” as used herein, refer to an amount or a concentration of one or more compounds or a pharmaceutical composition described herein utilized for a period of time (including in vitro and in vivo acute or chronic administration and periodic or continuous administration) that is effective within the context of its administration for causing an intended effect or physiological outcome.

Effective amounts of one or more compounds or a pharmaceutical composition for use in the present invention include amounts that promote beta cell survival or increase levels of insulin production, or a combination thereof.

The term “subject” is used throughout the specification to describe an animal, human or non-human, to whom treatment according to the methods of the present invention is provided.

The beta cells used in the invention can be derived from a pluripotent stem cells of a subject with diabetes. Such pluripotent stem cells can be obtained by a number of methods such as the illustrative method shown herein, which is by iPSC.

By “pluripotent stem cells”, it is meant cells that can a) self-renew and b) differentiate to produce all types of cells in an organism. The term “induced pluripotent stem cell” encompasses pluripotent stem cells, that, like embryonic stem (ES) cells, can be cultured over a long period of time while maintaining the ability to differentiate into all types of cells in an organism, but that, unlike ES cells (which are derived from the inner cell mass of blastocysts), are derived from somatic cells, that is, cells that had a narrower, more defined potential and that in the absence of experimental manipulation could not give rise to all types of cells in the organism. iPS cells have an hESC-like morphology, growing as flat colonies with large nucleo-cytoplasmic ratios, defined borders and prominent nuclei. In addition, iPS cells express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. In addition, the iPS cells are capable of forming teratomas. In addition, they are capable of forming or contributing to ectoderm, mesoderm, or endoderm tissues in a living organism.

In one embodiment, the invention provides a method of identifying a compound that is an ER stress reliever. The compound can be a small molecule, a nucleic acid (e.g., DNA or RNA), antisense, RNAi, peptide, polypeptide, mimetic and the like. The method includes contacting a beta cell, in vitro or in vivo, with a test compound and measuring the level of insulin produced prior to and following contacting with the test compound, wherein an increase in insulin levels after contacting is indicative of an ER stress reliever compound. In one aspect, the beta cell is derived from a subject having diabetes. In a particular aspect, the beta cell is derived from a pluripotent stem cell of a subject having diabetes. The beta cell can be derived from differentiation of a pluripotent stem cell, for example, using iPSC.

The beta cells of the invention can be derived by various methods using for example, adult stem cells, embryonic stem cells (ESCs), epiblast stem cells (EpiSCs), and/or induced pluripotent stem cells (iPSCs; somatic cells that have been reprogrammed to a pluripotent state). Illustrative iPSCs are stem cells of adult origin into which the genes Oct-4, Sox-2, c-Myc, and Klf have been transduced, as described by Takahashi and Yamanaka (Cell 126(4):663-76 (2006)). Other exemplary iPSC's are adult stem cells into which OCT4, SOX2, NANOG, and LIN28 have been transduced (Yu, et al., Science 318:1917-1920 (2007)). One of skill in the art would know that a cocktail of reprogramming factors could be used to produce iPSCs such as factors selected from the group consisting of OCT4, SOX2, KLF4, MYC, Nanog, and Lin28. Further, the methods described herein for producing iPSCs are illustrative of the method of the present invention for deriving beta cells.

Differentiation of pluripotent stem cells may be monitored by a variety of methods known in the art. Changes in a parameter between a stem cell and a differentiation factor-treated cell may indicate that the treated cell has differentiated. Microscopy may be used to directly monitor morphology of the cells during differentiation. As an example, the differentiating pancreatic cells may form into aggregates or clusters of cells. The aggregates/clusters may contain as few as 10 cells or as many as several hundred cells. The aggregated cells may be grown in suspension or as attached cells in the pancreatic cultures.

Changes in gene expression may also indicate beta cell differentiation. Increased expression of beta cell-specific genes may be monitored at the level of protein by staining with antibodies. Antibodies against insulin, Glut2, Igf2, islet amyloid polypeptide (IAPP), glucagon, neurogenin 3 (ngn3), pancreatic and duodenal homeobox 1 (PDX1), somatostatin, c-peptide, and islet-1 may be used. Cells may be fixed and immunostained using methods well known in the art. For example, a primary antibody may be labeled with a fluorophore or chromophore for direct detection. Alternatively, a primary antibody may be detected with a secondary antibody that is labeled with a fluorophore, or chromophore, or is linked to an enzyme. The fluorophore may be fluorescein, FITC, rhodamine, Texas Red, Cy-3, Cy-5, Cy-5.5. Alexa.sup.488, Alexa.sup.594, QuantumDot.sup.525, QuantumDot.sup.565, or QuantumDot.sup.653. The enzyme linked to the secondary antibody may be HRP, beta-galactosidase, or luciferase. The labeled cell may be examined under a light microscope, a fluorescence microscope, or a confocal microscope. The fluorescence or absorbance of the cell or cell medium may be measured in a fluorometer or spectrophotomer.

Changes in gene expression may also be monitored at the level of messenger RNA (mRNA) using RT-PCR or quantitative real time PCR. RNA may be isolated from cells using methods known in the art, and the desired gene product may be amplified using PCR conditions and parameters well known in the art. Gene products that may be amplified include insulin, insulin-2, Glut2, Igf2, LAPP, glucagon, ngn3, PDX1, somatostatin, ipfl, and islet-1. Changes in the relative levels of gene expression may be determined using standard methods. The expression of alpha-, beta-, gamma-, and delta-cell specific markers may show that the cell populations are composed of all four distinct types and three major types of pancreatic cells.

The compounds of the invention, together with a conventionally employed adjuvant, carrier, diluent or excipient may be placed into the form of pharmaceutical compositions and unit dosages thereof, and in such form may be employed as solids, such as tablets or filled capsules, or liquids such as solutions, suspensions, emulsions, elixirs, or capsules filled with the same, all for oral use, or in the form of sterile injectable solutions for parenteral (including subcutaneous use). Such pharmaceutical compositions and unit dosage forms thereof may comprise ingredients in conventional proportions, with or without additional active compounds or principles, and such unit dosage forms may contain any suitable effective amount of the active ingredient commensurate with the intended daily dosage range to be employed.

When employed as pharmaceuticals, the sulfonamide derivatives of this invention are typically administered in the form of a pharmaceutical composition. Such compositions can be prepared in a manner well known in the pharmaceutical art and comprise at least one active compound. Generally, the compounds of this invention are administered in a pharmaceutically effective amount. The amount of the compound actually administered will typically be determined by a physician in the light of the relevant circumstances, including the condition to be treated, the chosen route of administration, the actual compound administered, the age, weight, and response of the individual patient, the severity of the patient's symptoms, and the like.

The pharmaceutical compositions of these inventions can be administered by a variety of routes including oral, rectal, transdermal, subcutaneous, intravenous, intramuscular, intrathecal, intraperitoneal and intranasal. Depending on the intended route of delivery, the compounds are preferably formulated as either injectable, topical or oral compositions. The compositions for oral administration may take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampoules or syringes of the liquid compositions or pills, tablets, capsules or the like in the case of solid compositions. In such compositions, the sulfonamide compound is usually a minor component (from about 0.1 to about 50% by weight or preferably from about 1 to about 40% by weight) with the remainder being various vehicles or carriers and processing aids helpful for forming the desired dosing form.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatine; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As above mentioned, the sulfonamide derivatives of formula I in such compositions is typically a minor component, frequently ranging between 0.05 to 10% by weight with the remainder being the injectable carrier and the like.

The above described components for orally administered or injectable compositions are merely representative. Further materials as well as processing techniques and the like are set out in Part 5 of Remington's Pharmaceutical Sciences, 20th Edition, 2000, Marck Publishing Company, Easton, Pa., which is incorporated herein by reference.

The compounds of this invention can also be administered in sustained release forms or from sustained release drug delivery systems. A description of representative sustained release materials can also be found in the incorporated materials in Remington's Pharmaceutical Sciences.

The compounds of the invention can be co-administered with insulin, either prior to, simultaneously with or following administration of invention compounds. Insulin is a polypeptide composed of 51 amino acids which are divided between two amino acid chains: the A chain, with 21 amino acids, and the B chain, with 30 amino acids. The chains are linked together by two disulfide bridges. Insulin preparations have been employed for many years in diabetes therapy. Such preparations use not only naturally occurring insulins but also, more recently, insulin derivatives and insulin analogs.

Insulin analogs are analogs of naturally occurring insulins, namely human insulin or animal insulins, which differ by replacement of at least one naturally occurring amino acid residue by other amino acids and/or by addition/deletion of at least one amino acid residue, from the corresponding, otherwise identical, naturally occurring insulin. The amino acids in question may also be amino acids which do not occur naturally.

Insulin derivatives are derivatives of naturally occurring insulin or an insulin analog which are obtained by chemical modification. The chemical modification may consist, for example, in the addition of one or more defined chemical groups to one or more amino acids. Generally speaking, the activity of insulin derivatives and insulin analogs is somewhat altered as compared with human insulin.

The following examples illustrate the present invention, and are set forth to aid in the understanding of the invention, and should not be construed to limit in any way the scope of the invention as defined in the claims which follow thereafter.

### EXAMPLES

### Example 1

### Production of Insulin Producing Cells

The protocol for producing insulin producing cells is as follows:

Human embryonic stem cells or induced pluripotent stem cells are cultured under standard procedures and conditions that are known in the art. Prior to differentiation, cells are detached and dissociated using Dispase (3-5 mM @ RT) and, subsequently, Accutase (3-5 mM @ RT). Cells are suspended in human ES medium with ROCK inhibitor (Y27632) and filtered through 70 um (or 100 um) cell strainer. After that, cells are seeded a density of 400,000-800,000 cells/well (6-well plate) or 200,000-400,000 cell/well (12-well plate) or 50,000-200,000 cell/well (24-well plate) or 25,000-50,000 cell/well (96-well). Cells are kept grown for 1 or 2 days (the culture should be confluent).

On Day 1, cells are washed once with RPMI medium (with 1× Pen-Strep, 1× Glutamax). Then cells are cultured in RPMI medium (with 1× Pen-Strep, 1× Glutamax) containing human Activin A protein (100 ng/ml), human Wnt3A protein (25 ng/ml) and 0.15 mM Ethylene glycol-bis(2-aminoethylether)-N,N,N′,N′-tetraacetic acid.

On Day 2 and 3, cells are cultured in RPMI medium (with 1× Pen-Strep, 1× Glutamax) containing human Activin A protein (100 ng/ml) and 0.2% FBS in RPMI medium (with 1× Pen-Strep, 1× Glutamax).

On Day 4 and 5: cells are cultured in RPMI medium (with 1× Pen-Strep, 1× Glutamax) containing human FGF 10 protein (50 ng/ml), KAAD-cyclopamine (0.25 uM) and 2% FBS.

On Day 6, 7 and 8, cells are cultured in DMEM (high glucose) medium (with 1× Pen-Strep, 1× Glutamax) containing human FGF10 protein (50 ng/ml), KAAD-cyclopamine (0.25 uM), retinoic acid (2 uM) and LDN-193189 (250 nM) and 1×B27.

On Day 9 and 10, cells are cultured in CMRL medium (with 1× Pen-Strep, 1× Glutamax) containing exedin-4 (50 ng/ml), SB431542 (2 uM) 1×B27.

On Day 11 and 12, cells are culture in CMRL medium (with 1× Pen-Strep, 1× Glutamax) containing 4-(4-Hydroxy-3,5-diiodophenoxy)-3,5-diiodobenzeneacetic acid (20 pM) and 1×B27.

Cells can be maintained in CMRL medium (with 1× Pen-Strep, 1× Glutamax) containing 1×B27.

### Example 2

### Characterization of Insulin Producing Cell Functionality

The disclosure provides a method to characterize the functionality of above mentioned insulin-producing pancreatic cells by measuring insulin secretion in response to stimuli including glucose and potassium. Insulin-producing cells are washed in CMRL medium containing 5.6 mM glucose for one hour. Then cells are incubated in CMRL medium with 5.6 mM glucose for one hour and the medium is collected. Then, cells are incubated in CMRL medium containing 16.9 mM glucose or 35 mM potassium for one hour and the medium is collect. The levels of human c-peptide in the media are measured as indicator of insulin secretion.

### Example 3

### Transplantation of Pancreatic Progenitor Cells

The disclosure provides a method to transplant abovementioned pancreatic progenitor cells into mice. Insulin-producing cells are digested by trypsin and suspended in CMRL medium for 12-24 hours. The cells are collected and 2×10⁶ cells are transplanted under the kidney capsule of one NSG mouse.

### Example 4

### Functionality of Transplanted Cells

The disclosure provides a method to access functionality of cells transplanted into mice by measuring insulin secretion in response to glucose. Mice are deprived of food overnight (12-14 hrs), but have water available. In the morning, each mouse is weighed, injected intraperitoneally with a glucose solution (in saline, 1 mg/g body weight) and put into an empty cage (no food or water). Every half an hour the mouse is analyzed for blood glucose level by tail vein bleeding. Urine glucose concentration is also examined Blood samples are collect right before glucose injection and after half an hour of glucose injection. Human c-peptide levels in the blood serum are measured.

The disclosure provides methods to investigate causal factors of beta cell phenotypes in diabetes comprising evaluating functionality of insulin-producing cells in the dish and transplanted cells in mice. The cells with mutations in the genes relate to beta cell development and/or functionality show defects in insulin secretion in response to circulating glucose concentrations.

Further applications of the disclosure include screening for small molecules that will attenuate phenotypes of beta cells with genetic defects causing beta cell dysfunction, and screening for molecules that will enhance the efficiency of generation of insulin-producing cells from stem cells.

### Example 5

### Generation of Induced Pluripotent Stem Cells

Induced pluripotent cells suitable for use with the methods described herein can be generated by (a) obtaining a source cell by taking a skin biopsy from a mammal (e.g. a mouse or a human), (b) establishing a fibroblast cell line from the skin biopsy, (c) infecting the fibroblast cell line with a retrovirus or a Sendai virus capable of directing expression of human transcription factors Oct4, Sox2, Klf4 and C-Myc in the fibroblast cell line. In certain embodiments, one or more colonies of induced pluripotent stem cells can be isolated 3 weeks after infection with the retrovirus or Sendai virus. In certain embodiments, the isolated one or more colonies of induced pluripotent stem cells can be expanded to establish one or more induced pluripotent stem cells.

### Example 6

### Understanding Intrinsic Beta Cell Defects in Monogenic Forms of Diabetes

Recently developed patient-specific stem cells can be useful for the study of human genetics and diseases, including diabetes. To take the advantage of this technology and, at the same time, to assess its feasibility in the study of diabetes, models for monogenic diabetes with beta cell autonomous dysfunctions were generated. The model cells were examined to determine whether beta cells carrying genetic mutations show corresponding cellular and molecular pathologies.

Maturity-onset diabetes of the young (MODY), a subtype of monogenic forms of diabetes, is caused by single gene mutations that directly affect beta cell development and functions. While several defects are caused by mutations of transcription factors, MODY2, one of the most common forms of monogenic diabetes, results from functional hypomorphs of glucokinase (GCK). GCK serves as a glucose sensor for the beta cell and alterations of the activity of GCK can result in a glucose-sensing defect). Hypomorphic mutations of GCK (of which many have been identified) lead to chronic, mild hyperglycemia.

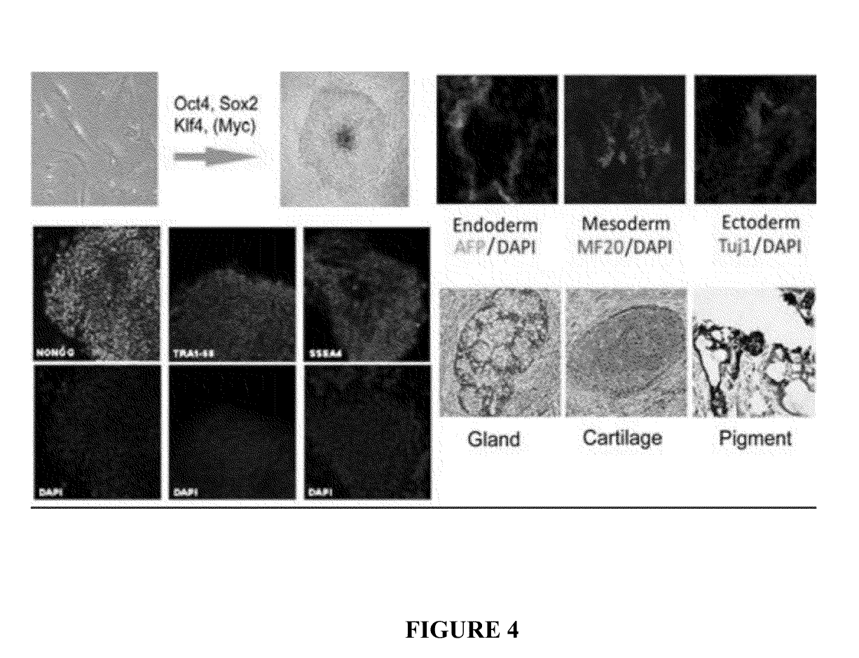

The methods described herein have been used to generate pluripotent stem cell lines from skin fibroblasts. Fibroblast cell lines and iPS cell lines were generated from two MODY2 patients with missense mutations in GCK. The pluripotency of these iPS cells was verified by immunocytochemistry, embryoid body and teratoma formation assays. The resulting embryoid bodies and teratomas contained cell types of three germ layers-endoderm, mesoderm and ectoderm (FIG. 4).

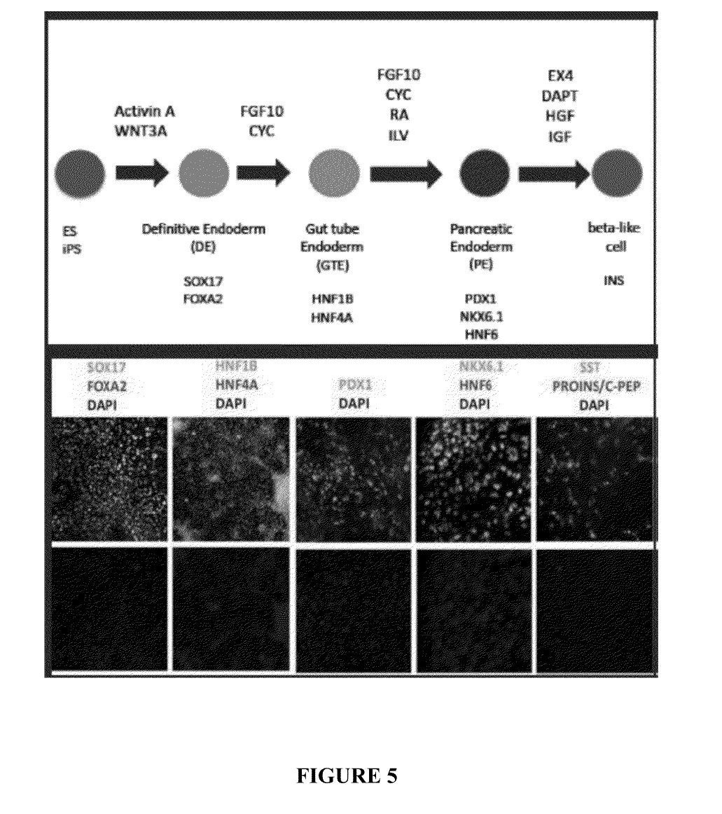

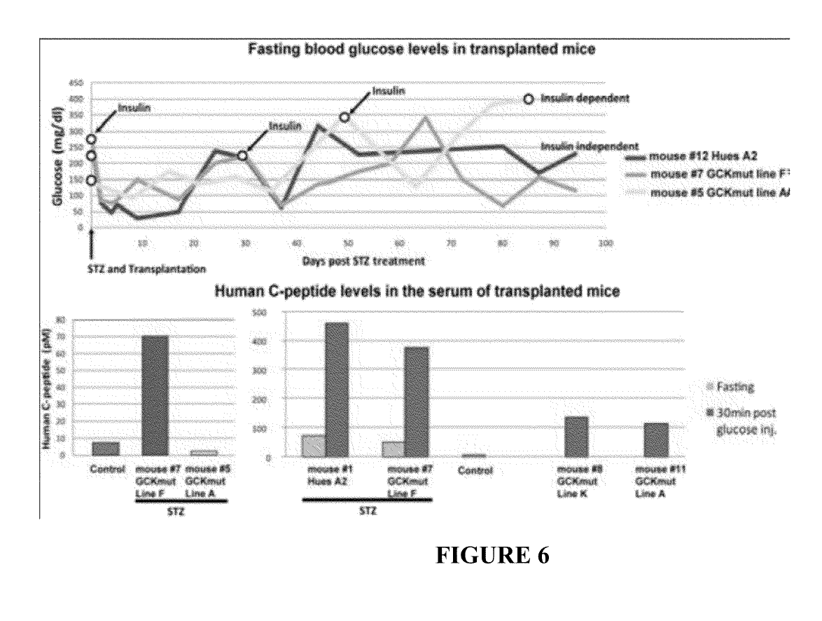

These stem cells were differentiated in vitro into beta-like cells (FIG. 5) and were also transplanted similarly derived pancreatic endoderm into immunocompromised mice as another means of promoting the differentiation of these cells into functioning beta cells (FIG. 6). These beta-like cells were able to produce and secrete insulin and could response to glucose in the culture dish and in mice. The results of this analysis show differentiation of patient-specific iPS cells toward pancreatic endoderm and insulin-producing cells in vitro and in vivo. The results of this analysis also show beta cells derived from patients are less responsive to glucose comparing to the cells from healthy controls. The results of this analysis further provide systems suitable for testing functionality of beta cells

### REFERENCES

1. Methods for increasing definitive endoderm production, U.S. Pat. No. 7,695,963
2. D'Amour, K. A., Bang, A. G., Eliazer, S., Kelly, O. G., Agulnick, A. D., Smart, N. G., Moorman, M. A., Kroon, E., Carpenter, M. K., and Baetge, E. E. (2006). Production of pancreatic hormone-expressing endocrine cells from human embryonic stem cells. Nat Biotechnol 24, 1392-1401.
3. Kroon, E., Martinson, L. A., Kadoya, K., Bang, A. G., Kelly, O. G., Eliazer, S., Young, H., Richardson, M., Smart, N. G., Cunningham, J., et al. (2008). Pancreatic endoderm derived from human embryonic stem cells generates glucose-responsive insulin-secreting cells in vivo. Nat Biotechnol 26, 443-452.

### Example 7

### A Stem Cell Model of Diabetes Due to Glucokinase Deficiency

Diabetes is a disorder characterized by loss of beta cell mass and/or beta cell function, leading to deficiency of insulin relative to metabolic need. To determine whether stem cell derived beta cells faithfully reflect the phenotype of a diabetic subject, we generated stem cells from diabetic subjects (MODY2) with heterozygous loss-of-function of the gene encoding glucokinase (GCK). We found that heterozygous GCK mutations reduced glucose-responsive insulin secretion in stem cell derived beta cells in vitro as well as in vivo after transplantation into mice. Compound heterozygous GCK mutations reduced the number of insulin-producing cells generated from iPSCs, suggesting a role of GCK in beta cell proliferation. Importantly these phenotypes were fully reverted upon gene sequence correction by homologous recombination. Our results demonstrate that stem cell-derived beta-like cells accurately reflect systemic phenotypes of MODY 2 subjects, providing a platform for mechanistic analysis of the pathogenesis of more prevalent types of diabetes.

Recent progress in somatic cell reprogramming has allowed the generation of induced pluripotent stem (iPS) cells from diabetic subjects (1). iPS cells and human embryonic stem cells have the capacity to differentiate into insulin-producing cells (2), which present key properties of true beta cells, including glucose-stimulated insulin secretion (3). However, whether in vitro derived insulin-producing cells can faithfully replicate pathologic phenotypes, be used to evaluate the functional relevance of disease mechanisms, and to test strategies to restore normal beta cell function is not clear. As proof-of-principle, we chose to model a monogenic form of diabetes, MODY2.

Maturity-onset diabetes of the young (MODY) is caused by single gene mutations, resulting in defects in the development, proliferation/regeneration, and/or function of beta cells (4). MODY accounts for 1 to 5 percent of all instances of diabetes in the United States (5), and MODY2 (the most prevalent) accounts for 8-60% of all MODY cases, depending on ethnicity (6, 7).

Glucokinase links blood glucose levels to insulin secretion by converting glucose to glucose-6-phosphate, the rate-limiting step in glycolysis. The catalytic capacity of glucokinase (GCK) in beta cells determines the threshold for glucose stimulated insulin secretion. The normal threshold for glucose-stimulated insulin secretion is ˜5 mmol/l in healthy human subjects. In MODY2 subjects, this threshold is increased to ˜7 mmol/l due to hypofunction of one allele of GCK, resulting in mild hyperglycemia (8). The loss of both GCK alleles results in permanent neonatal diabetes (9). Conversely, activating mutations of GCK result in persistent hyperinsulinemic hypoglycemia, due to a decreased glucose threshold for insulin secretion (e.g. <3.7 mmol/l for mutation Y214C) (10). While the MODY2 systemic phenotypes, elevated blood glucose levels and delayed insulin secretion, are well characterized in human subjects (11), the consequences of the responsible mutations for detailed aspects of beta cell development and function are difficult to assess. For instance, whether GCK affects processes such as insulin biosynthesis or beta cell mass could not be determined. In a mouse model, heterozygous loss of GCK causes hyperglycemia, early-onset diabetes (10 weeks old), reduced beta cell response to glucose (12), and an inability to increase beta cell mass under conditions of insulin resistance (13). Complete lack of GCK in mouse pancreatic beta cells throughout development leads to marked glycosuria at birth and severe hyperglycemia and death from dehydration (12), which represents neonatal diabetes in human. Mouse islets with homozygous loss of GCK showed blunted response to glucose. The relevance of these different possible effects of GCK on the functionality of human beta cells is unclear. Patient-specific stem cells and beta cells generated from these patients could, potentially, be used to directly address these questions.

Here, we generated pancreatic hormone-expressing cells deficient for glucokinase due to missense mutations or targeted gene disruption. Induced pluripotent stem cells (iPSCs) from MODY2 subjects heterozygous for hypomorphic GCK mutations differentiated normally into insulin-producing pancreatic endocrine cells. In contrast, stem cells with two inactive GCK alleles showed a reduced capacity to generate insulin-producing cells. Hetero- or homozygosity for hypomorphic GCK alleles reduced insulin secretion in response to glucose in iPS-derived insulin-producing cells. Functional phenotypes resulting from GCK mutations were fully reverted after correction of the mutation by homologous recombination. These results demonstrate that the phenotypes of stem cell-derived patient-specific insulin producing cells recapitulate the functional phenotypes observed in vivo, and enable analysis of aspects of cellular physiology not otherwise possible.

Stem Cells with an Allelic Series at the GCK Locus

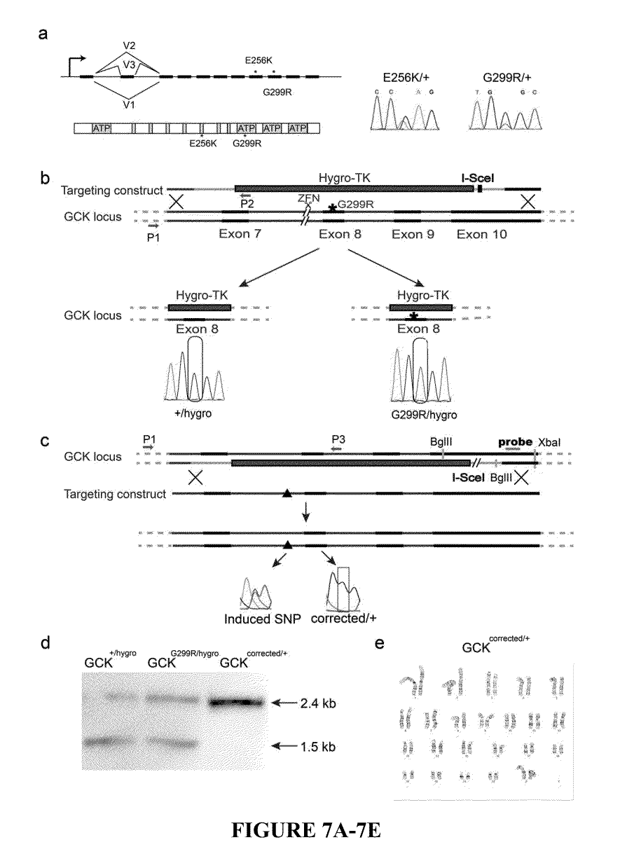

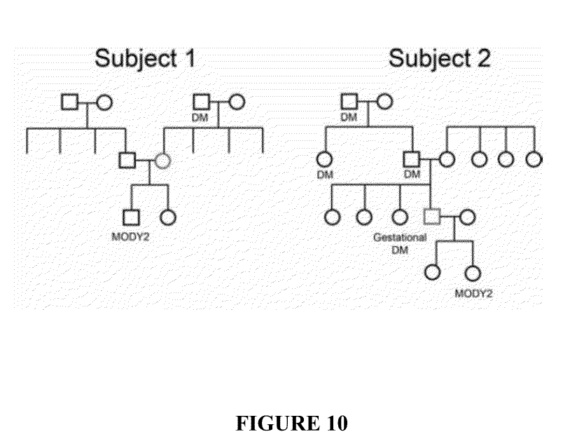

We obtained skin biopsies from two MODY2 subjects and established fibroblast cell lines. One subject is a 38 year old Caucasian female who was diagnosed with diabetes at the age of 21 years. The other subject is a 56 year old Caucasian male who was diagnosed with diabetes at age 47. Both of them were non-obese (BMI=21 to 26 kg/m²) and positive for measurable, but low serum C-peptide (0.1 to 0.4 ng/ml). Diabetes control was excellent (HbAlC's:S 6.5%) on insulin or sulfonylurea-related agents (Table 1). Due to their strong family history of diabetes (FIG. 10) and negative results for antibodies associated with type 1 diabetes, they underwent genetic testing. Exonic sequencing of GCK revealed that the female patient carries a missense mutation (G299R) in one of the ATP binding domains and the male patient has a missense mutation (E256K) in a substrate-binding site (FIG. 7A).

TABLE 1
Summary of clinical characteristics of the 2 MODY2 subjects

| | | Age at | | | | Family | Controlled |
|---|---|---|---|---|---|---|---|
| | Genetic | Diabetes | Anti-GAD | | | history of | with oral |
| | Diagnosis | Diagnosis | Antibodies | BMI | Race | diabetes | agents |
| Subject 1 | GCK | 21 | Neg | 21 | Caucasian | 3 | yes |
| | mutation | | | | | generations | |
| | gly229>arg | | | | | | |
| Subject 2 | GCK | 47 | Neg | 26 | Caucasian | 2 | yes |
| | mutation | | | | | generations | |
| | glu256>lys | | | | | | |

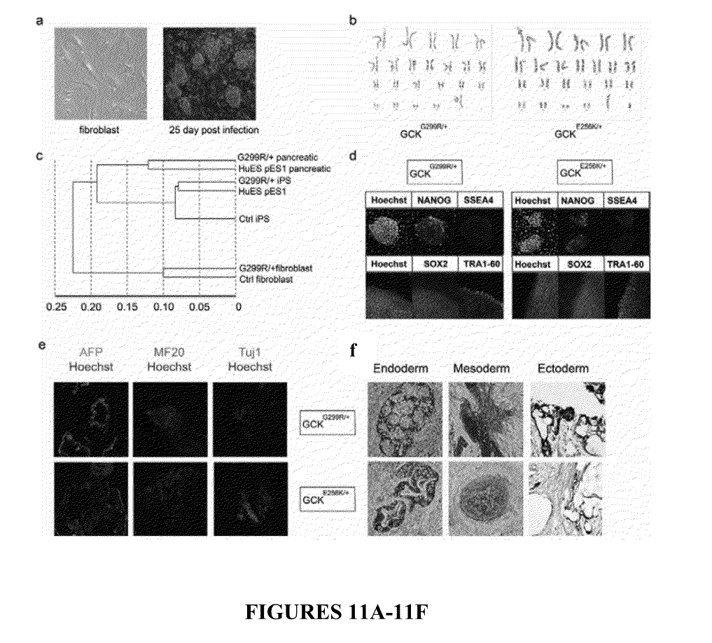

Induced pluripotent stem cell lines were generated using Sendai viruses containing Oct4, Sox2, Klf4 and c-Myc (14). The iPS cells with the hypomorphic GCK mutations indicated closely resembled human embryonic stem cells in their gene expression profiles and capability to differentiate (FIG. 11). Because of the genetic diversity in humans, the choice of appropriate comparison cells is critical for functional comparisons between mutant and non-mutant cells (15). In order to generate cell lines with identical genetic background, but with different genotypes at the GCK locus, we performed targeted genetic modifications (FIG. 7B). Homologous recombination in human stem cell lines has recently been made possible by the use of locus-specific “designer” nucleases (16, 17). We designed a two-step targeting protocol that allowed the precise correction of the mutant base pair without leaving a footprint of exogenous DNA. We first targeted the GCK locus with a linearized construct containing a PGK-hygro-TK fusion gene, flanked by two segments of the GCK locus corresponding to intron 6 and exon 10 in GCK^{G299R/+} cells. Messenger RNA encoding a zinc finger nuclease to induce a double-strand break (DSB) 1150 bp upstream of the G299R mutation was electroporated into GCK^{G299R/+} cells with the targeting plasmid to introduce a double strand break and facilitate homologous recombination. Hygromycin-resistant colonies of transfected GCK^{G299R/+} cells were expanded and tested for homologous integration using PCR primers annealing to the genomic sequence and to the hygro-TK cassette (FIG. 7B). Of 201 hygromycin-resistant colonies, 14 (7%) showed targeting of the construct to either the wild type or the mutant allele, resulting in GCK^{G299R/hygro} and GCK^{+/hygro} cells, respectively (FIG. 7C).

In a second step, to correct the mutant allele and eliminate all vector sequences, a plasmid containing the wild type GCK locus, but marked with a PCR-induced polymorphism (‘induced SNP’ in FIG. 7C), was transfected into GCK^{+/hygro} cells. A plasmid encoding the endonuclease I-SceI site was co-transfected to induce a DSB at the I-SceI recognition site located in the hygro-TK cassette to facilitate homologous recombination (FIG. 7B). Ganciclovir-resistant colonies would have lost the hygro-TK cassette either due to homologous recombination or non-homologous end joining. Using PCR with one primer outside of the targeting construct and one primer within the construct, followed by sequencing of the induced SNP, we selected for homologous integration events. 2 of 96 colonies (2% efficiency) contained the induced polymorphism targeted to the GCK locus (FIG. 7D); these were designated GCK^{corrected/+} cells. We performed Southern blotting to confirm that GCK^{corrected/+} cells contained two wild type alleles at the GCK locus, indicated by a single band of wild type size (FIG. 7E). These targeted manipulations resulted in an allelic series of cells that were wild type (GCK^{corrected/+}), hypomorph (GCK^{G299R/+}) and or null (GCK^{G299R/hygro}) for GCK function on the same genetic background, allowing us to exclude potential confounding effects of different genetic backgrounds in subsequent experiments.

Efficient Beta Cell Generation from GCK Deficient iPS Cells

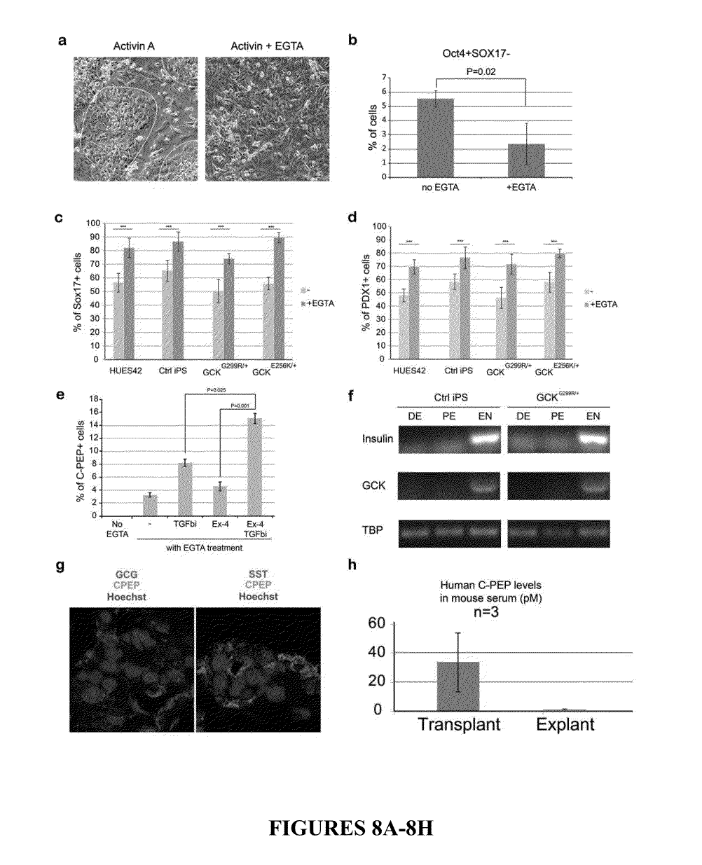

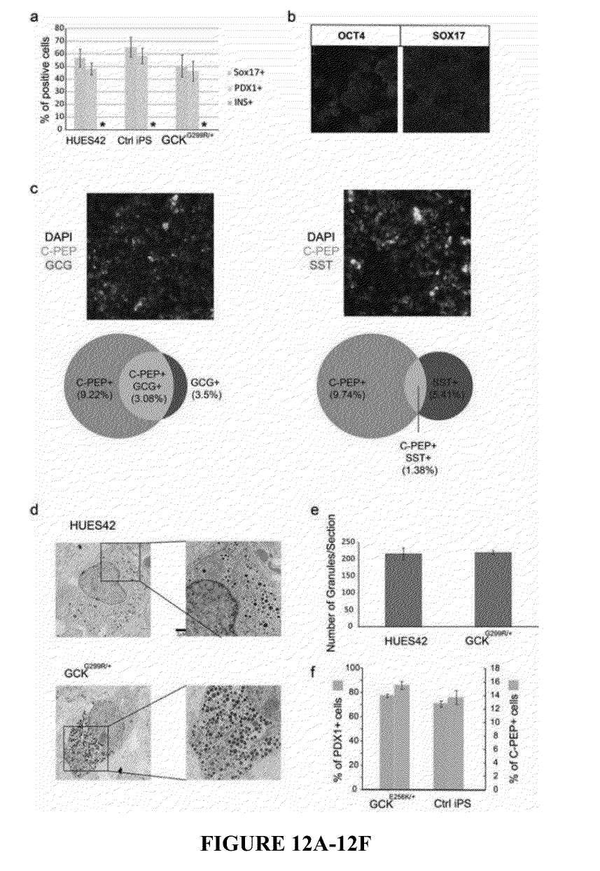

Human embryonic stem cells and iPS cells can be differentiated towards insulin producing cells after stepwise differentiation into definitive endoderm (SOX17+), pancreatic progenitors (PDX1+) and endocrine progenitors (NGN3+) (2, 18). While the published protocols were sufficient to yield Sox17− and Pdx1− positive cells, the efficiency was low and differed greatly among different iPS cell lines, and insulin-producing cells were not obtained (FIG. 12A). We noticed that 3 days after induction of differentiation, large colonies with the morphology of pluripotent stem cells were still apparent. These cells retained Oct4 expression and failed to commit to the endoderm lineage, as evidenced by the lack of Sox17 expression (FIG. 12B). We reasoned that interfering with the maintenance of pluripotency should increase the efficiency of differentiation. Cell-to-cell interactions mediated by E-cadherin are critical for maintaining pluripotency of ES cells (19). In addition, E-cadherin is down-regulated during the epithelial-mesenchymal transition, occurring in vivo during differentiation into definitive endoderm (20). We found that when the calcium chelator, EGTA, an inhibitor of cadherins (21), was applied to stem cells on the first day of differentiation, less cell-cell contact was reflected by the loss of tight colony morphology (FIG. 8A); the percentage of OCT4+SOX17− control iPS cells was also reduced from 5% to 2% (FIG. 8B) and the percentage of endodermal (50×17+ OCT4−) cells was increased by 25.5% (FIG. 8C). This directly translated into a 21.7% increase in Pdx1 positive cells on day 8 of differentiation. The addition of EGTA also reduced the variability between cell lines: cell lines that had performed poorly without addition of EGTA (<50% PDX1+) showed a high yield of PDX1+ progenitor cells with the addition of EGTA (>70%) (FIG. 8D). To further improve differentiation conditions from pancreatic progenitor to beta-like cells, exendin-4 and SB431542, a TGFbeta signaling inhibitor, were added to progenitors. Both of these additions enhanced the differentiation efficiency of beta-like cells to 4.6% and 8.2% (C-PEP+), respectively, consistent with previous observations (22-24). A combination of exendin-4 and SB431542 produced the highest percentage of beta-like cells (15%) (FIG. 8E). We observed that 38% of the insulin-producing cells also immunostained for glucagon and 14% of the insulin-producing cells also expressed somatostatin, similar to previous observations (18) (FIG. 12C). Further differentiation into monohormonal cells occurred in vivo, after transplantation of pancreatic progenitor cells under the kidney capsule of immune-compromised mice. Three months after transplantation, 24 of 50 mice had detectable human C-peptide in their serum Immunohistochemistry of the isolated graft showed that hormone-expressing cells in the transplants expressed solely insulin, glucagon or somatostatin (FIG. 8G). To determine whether the C-peptide originated from the transplants, we removed the transplants from 7 mice, and found that none retained detectable human C-peptide in serum (FIG. 8H).

Heterozygous GCK Mutations Specifically Affect Glucose Mediated Insulin Secretion

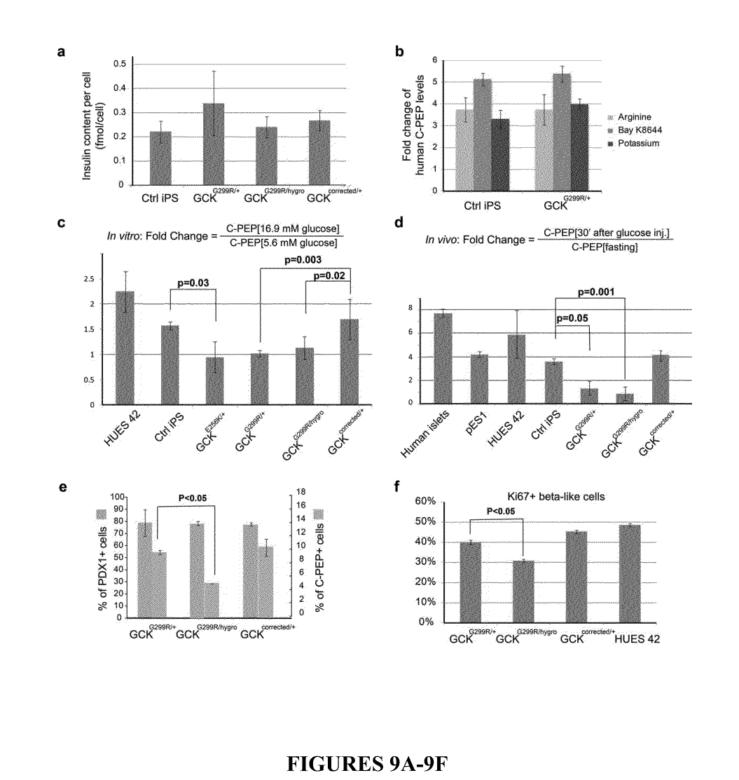

We assessed the temporal expression pattern of GCK during the in vitro differentiation process. We measured GCK mRNA levels at definitive endoderm (day 3), pancreatic endoderm (day 8) and endocrine (day 12) stages. Expression of GCK was detected only at the endocrine stage, coinciding with the expression of insulin (FIG. 8F). GCK mutations could affect beta cell function by reducing insulin production/processing, or by interfering with insulin secretion in response to glucose, or to glycolysis-independent secretagogues. These possibilities are not mutually exclusive. We found that insulin content was comparable in control beta-like cells and cells with genotype of GCK^{G299R/+}, GCK^{G299R/hygro} and GCK^{corrected/+} (FIG. 9A). By electron microscopy, cellular granule morphology and numbers were comparable in wild type (average 173 granules per cross-section) and GCK^{G299R/+} (average 220 granules per cross-section) (FIG. 12D, E). If GCK effects are mediated solely by glucose sensing, insulin secretion in response to secretagogues acting independent of glycolysis should be unaffected.

We found that GCK^{G299R/+} cells responded to arginine (3-4 fold), potassium (3-4 fold), and to Bay K8644, a calcium channel agonist (5-fold) increases in C-peptide release, identical to control cells (FIG. 9B). Beta-like cells derived from human ES cells and control iPS cells showed 1.5-2 fold increase in C-peptide secretion when ambient glucose concentrations were increased from 5.6 mM (physiological) to 16.9 mM. In contrast, GCK^{E256K/+}, GCK^{G299R/+} and GCK^{G299R/hygro} cells showed no increase. Importantly, correction of the G299R mutation to the wild type nucleotide sequence, restored glucose responsiveness: GCK^{corrected/+} cells showed a 1.7-fold increase in glucose-stimulated C-peptide secretion (FIG. 9C). To determine if these differences between control and GCK mutant cells were also present in vivo, we performed intraperitoneal glucose tolerance tests on transplanted mice. Human islets, and beta-like cells derived from human ES and control iPS cells showed a 4 to 6-fold increase in serum human C-peptide concentrations upon glucose administration. In contrast, GCK^{G299/+} cells showed only a 2-fold increase in serum C-peptide concentration, and GCK^{G299R/hygro} cells showed no increase. The gene-corrected cells showed a 4-fold induction in C-peptide release (FIG. 9D, Table 2). Taken together, these results demonstrated that GCK mutations specifically affect glucose-mediated insulin secretion.

TABLE 2
Circulating human C-peptide levels in the transplanted mice. 300 islets
or 2-3 million in vitro differentiated cells were transplanted into each
mouse. Some iPS-derived

| | Human c-peptide levels (pM) | | |
|---|---|---|---|
| Transplanted cells | Prior to glucose inj. | 30 min after glucose inj. | Ratio |
| Human islets | 66.0 | 538.5 | 8.2 |
| Human islets | 55.0 | 413.4 | 7.5 |
| Human islets | 43.1 | 323.9 | 7.5 |
| pES1 | 24.5 | 104.2 | 4.3 |
| pES1 | 9.5 | 42.8 | 4.5 |
| pES1 | 31.1 | 124.1 | 4.0 |
| HUES 42 | 35.7 | 160.0 | 4.5 |
| HUES 42 | 43.5 | 223.4 | 5.1 |
| HUES 42 | 27.3 | 225.6 | 8.3 |
| Ctrl iPS | 22.3 | 87.2 | 3.9 |
| Ctrl iPS | 21.0 | 75.8 | 3.6 |
| Ctrl iPS | 40.7 | 140.3 | 3.4 |
| GCK^{G299R/+} | 69.0 | 152.4 | 2.2 |
| GCK^{G299R/+} | 12.2 | 13.8 | 1.1 |
| GCK^{G299R/+} | 44.5 | 34.4 | 0.8 |
| GCK^{G299R/+} | 39.4 | 54.54 | 1.4 |
| GCK^{G299R/hygro} | 80.2 | 64.0 | 0.8 |
| GCK^{G299R/hygro} | 58.8 | 90.7 | 1.5 |
| GCK^{G299R/hygro} | 51.2 | 19.8 | 0.4 |
| GCK^{corrected/+} | 58.5 | 240.1 | 4.11 |
| GCK^{corrected/+} | 66.0 | 251.9 | 3.81 |

implants produced amounts of human C-peptide that were comparable to those produced by the human islet implants.

Compound Heterozygous Mutations in GCK Affect Beta Cell Proliferation

The relatively late expression of GCK in beta cell development that we observed (FIG. 8F) suggests that GCK mutations should not affect the generation of pancreatic progenitors. Indeed, when we differentiated GCK^{G299R/+}, GCK^{G299R/hygro} and GCK^{corrected/+} cells into pancreatic endoderm, we found that all showed identical efficiency in generating pancreatic progenitors (PDX1+) (FIG. 9E). However, when we further differentiated the progenitor cells into beta-like cells, a significant reduction in beta-like cell generation was observed in GCK^{G299R/hygro} cells (5% C-peptide positive), compared to heterozygous loss of GCK (GCK^{G299R/+}) (10%) and gene-corrected cells (GCK^{corrected/+}) (10%) (FIG. 9E). This could be caused by defect in either differentiation of progenitor cells or proliferation of beta-like cells. The fact that we observed a reduction of Ki67 positive beta-like cells (31% of C-peptide positive cells) in the GCK^{G299R/hygro} genotype, compared to the genotypes GCK^{G299R/+} (41%), GCK^{corrected/+} (45%) and control GCK^{+/+} (HUES42, 49%) (FIG. 9F) suggests a defect of cell replication due to lack of GCK. GCK^{E256K/+} cells (15% c-peptide positive), like GCK^{G299R/+} cells, did not show any significant reduction in rates of cell division of beta-like cells compared to control iPS-derived cells (FIG. 12F).

Discussion

In this study, we tested the fidelity with which known cell-autonomous beta cell defects in a monogenic form of beta cell dysfunction are reflected by iPS-derived insulin-producing cells. We found that the ability of stem cell-derived beta cells to respond to glucose depended on gene dosage of functional GCK alleles: beta cells heterozygous for a hypomorphic GCK mutation, generated from stem cells with a MODY2 genotype, showed reduced insulin secretion in response to glucose, but not to other insulin secretagogues. And iPS-derived beta cells deficient for both alleles showed no glucose-stimulated insulin secretion. Therefore, stem cell derived beta cells recapitulate key aspects of the MODY2 phenotype, or of permanent neonatal diabetes, caused by the absence of one or both GCK alleles, respectively (9). These observations validate the concept of using stem cell-derived beta cells for disease modeling. We also found that beta-like cells carrying two inactive GCK alleles, but not cells with one or two functional GCK alleles, showed reduced rates of replication in vitro. Reduced replication of beta-like cells in addition to impaired glucose responsiveness suggests that beta cell mass may be reduced in cases of permanent neonatal diabetes. Consistent with this inference, Porat and colleagues recently demonstrated a role for GCK in regulating beta cell proliferation in adult mice (25). Because of the difficulty in accessing patient tissues, beta cell mass has not directly been determined in subjects with GCK mutations. Indications that beta cell mass or insulin production might be affected in neonatal diabetes are indirect: insulin release in response to sulfonylurea is insufficient to maintain glucose homeostasis in patients with neonatal diabetes due to GCK mutations (26). Meanwhile, MODY2 subjects, including one of our subjects, are typically well managed clinically with sulfonylurea therapy (i.e. glipizide or glyburide). Our results imply that both defective glucose-stimulated insulin secretion and reduced beta cell replication may contribute to the hypoinsulinemia resulting from homozygosity for hypomorphic GCK alleles, while in MODY2 subjects, beta cell mass may be unaffected.

iPS-derived cells should enable novel insights into the molecular-cell biology of beta cell failure in virtually all forms of diabetes. Stem cell models of other monogenic forms of diabetes, such as neonatal diabetes caused by mutations in KCNJ11, or Wolfram syndrome, caused by mutations in WFS1, should not only allow deeper insight into the relevant mutation-specific molecular cell biology, but in doing so, also shed light on the molecular physiology of the beta cell in other, more prevalent clinical circumstances. Common variants of WFS1 (27), KCNJ11 (28), and GCK (29, 30) increase the risk of T2D diabetes. Stem cell-based approaches will permit analysis of the molecular basis for these associations, and allow the investigation of genes modifying penetrance of specific mutations that affect beta cell function. Importantly, we were also able to demonstrate that the specific correction of the mutant base pair in the GCK locus by homologous recombination restores normal beta cell function. The generation of autologous beta cells in combination with gene correction may ultimately be useful for cell replacement to restore normal glucose homeostasis.

Materials and Methods:

Research Subjects and Cell Lines

Biopsies of upper arm skin were obtained from two subjects using local anesthesia (lidocaine) and an AcuPunch biopsy kit (Acuderm Inc). Samples were coded and transported to the laboratory. Biopsies were cut in 10-12 small pieces, and 2-3 pieces of minced skin were placed around a silicon droplet in a well of six-well dish. A glass cover slip was placed over the biopsy pieces and 5 ml of biopsy plating media were added. After 5 days, biopsy pieces were grown in culture medium for 3-4 weeks. Biopsy plating medium was composed of DMEM, FBS, GlutaMAX, Anti-Anti, NEAA, 2-Mercaptoethanol and nucleosides (all from Invitrogen) and culture medium contained DMEM, FBS, GlutMAX and Pen-Strep (all from Invitrogen). All studies were approved by the Columbia IRB and ESCRO committees. All Research subjects gave informed written consent.

Generation of Induced Pluripotent Stem Cells

Primary fibroblasts were converted into pluripotent stem cells using CytoTune™-iPS Sendai Reprogramming Kit (Invitrogen). 50,000 fibroblast cells were seeded per well of a six-well dish at passage three and allowed to recover overnight. Next day, Sendai viruses expressing human transcription factors Oct4, Sox2, Klf4 and C-Myc were mixed in fibroblast medium to infect fibroblast cells according to the manufacturer's instructions. Two days later, the medium was exchanged to human ES medium supplemented with the ALK5 inhibitor SB431542 (2 μM; Stemgent), the MEK inhibitor PD0325901 (0.5 μM; Stemgent), and thiazovivin (0.5 μM; Stemgent). Human ES medium contained KO-DMEM, KSR, GlutMAX, NEAA, 2-Mercaptoethanol, PenStrep and bFGF (all from Invitrogen). On day 7-10 post infection, cells were detached using TrypLE and passaged onto feeder cells. Individual colonies of induced pluripotent stem cells were picked between days 21-28 post infection and each iPS cell line was expanded from a single colony. All iPS cells lines were cultured on mouse embryonic fibroblast cells with human ES medium. Karyotyping was performed by Cell Line Genetics Inc. For teratoma analysis, 1-2 million cells from each iPS cell line were detached and collected after TrypLE (Invitrogen) treatment. Cells were suspended in 0.5 ml of human ES media. The cell suspension was mixed with 0.5 ml metrigel (BD Biosciences) and injected subcutaneously into dorsal flanks of an immunodeficient mouse (Stock No.:005557, The Jackson Laboratory). 8-12 weeks after injection, teratomas were harvested, fixed overnight with 4% paraformaldehyde and processed according to standard procedures for paraffin embedding. The samples were then sectioned and HE (hematoxylin and eosin) stained.

Gene Expression Analysis

Total RNA was isolated from cells with RNAeasy kit (Qiagen). For quantitative PCR analysis, cDNA was synthesized using Promega RT system (Promega). Primers for qRT-PCR were listed in Table 3. For microarray analysis, RNA was prepared using Illumina Total Prep RNA amplification kit (Ambion). cDNA was synthesized hybridized to HumanHT-12 v4 Beadchip kit (Illumina) The global expression profiles of the samples were analyzed using GenomeStudio Softwre (Illumina) and a hierarchical cluster tree was generated based on the correlation coefficients between samples.

TABLE 3
Primer sequences

| | | SEQ |
|---|---|---|
| | | ID |
| primer | sequences | NO: |
| GCK-5arm-forward | ccgctcgagcggtgcatcttccagct | 10 |
| GCK-5arm-reverse | cccaagcttgggcaccttccctgcct | 11 |
| GCK-3arm-forward | ccgctcgagcgggctggaatcaatttcccaga | 12 |
| GCK-3arm-reverse | cggaattccgcgtgatgctgttccagagaa | 13 |
| GCK-correction-forward | ccgctcgagcggtccccaagacacttccacat | 14 |
| GCK-correction-reverse | ggactagtccataggcgttccactgacagg | 15 |
| P1 | gcatcttccagctcttcgac | 16 |
| P2 | ctaaagcgcatgctccagac | 17 |
| P3 | aggccctagtttcccatcc | 18 |
| Southern Probe forward | tccagatgctcctgtcagtg | 19 |
| Southern Probe reverse | gagccaaagcaattccacat | 20 |
| INS RTPCR forward | ttctacacacccaagacccg | 21 |
| INS RTPCR reverse | caatgccacgcttctgc | 22 |
| GCK RTPCR forward | ctgaacctcaaaccccaaac | 23 |
| GCK RTPCR reverse | tgccaggatctgctctacct | 24 |
| GLUT2 RTPCR forward | catgtgccacactcacacaa | 25 |
| GLUT2 RTPCR reverse | atccaaactggaaggaaccc | 26 |

Directed Differentiation into Beta-Like Cells

ES or iPS cells were dissociated using Dispase (3-5 min) and, subsequently, Accutase (5 min) Cells were suspended in human ES medium containing 10 uM ROCK inhibitor (Y27632) and filtered through 70 um cell strainer. Cells were then plated at a density of 400,000 cell/well in 12-well plates. After 1 or 2 days, when cells reached 80-90% confluency, differentiation was started. Detailed formulations of differentiation medium are listed in Table 4. Typically, cells were assayed between day 12 and day 16. For measuring proliferation rate, cells were assayed at day 12. Insulin contents were measured using Insulin ELISA kit (Mercodia).

TABLE 4
Beta-cell differentiation medium compositions.

| | | Basic | |
|---|---|---|---|
| Stage | Day | Medium | Supplement |
| Mesendoderm | 1 | RPMI | Activin A (100 ng/ml) |
| | | | Wnt3A (25 ng/ml) |
| | | | 75 uM EGTA |
| Definitive Endoderm | 2-3 | RPMI | Activin A (100 ng/ml), |
| | | | 0.2% FBS |
| Primitive Gut Tube | 4-5 | RPMI | FGF10 (50 ng/ml), |
| | | | KAAD-cyclopamine (0.25 uM) |
| | | | 2% FBS |
| Posterior Foregut | 6-8 | DMEM | FGF10 (50 ng/ml), |
| | | | KAAD-cyclopamine (0.25 uM) |
| | | | Retinoic acid (2 uM) |
| | | | LDN-193189 (250 nM) |
| | | | B27 |
| Pancreatic Endoderm | 9-10 | CMRL | Exendin-4 (50 ng/ml) |
| | | | SB431542 (2 uM) |
| | | | B27 |
| Endocrine | 11+ | CMRL | B27 |

Gene Targeting

A targeting vector (pBS-PGK-hytk-IsceI-LoxP) was constructed by cloning a PGK-hygro-TK cassette into a pBlueScript SK+ vector. A LoxP site was added 5′ of the cassette. A LoxP and an I-SceI site were cloned behind the 3′ end of the cassette. Two DNA fragments, “homologous arms”, from the glucokinase (GCK) gene (see Table 3 for primer sequences) were cloned into the pBS-PGK-hytk-IsceI-LoxP vector at 5′ and 3′ end of the cassette. A correction construct was created by cloning a DNA fragment of GCK (see Table 3 for primer sequences) into pCR2.1-TOPO vector using TOPO TA cloning kit (Invitrogen).

A pair of zinc-finger nucleases (ZFN) was designed by Sigma to recognize the following sequence in intron 7 of GCK: CGTCAATACCGAGTGgggcgcCTTCGGGGACTCCGGC (UPPERCASE: ZFN-binding site, lowercase: cut site) (SEQ ID NO: 27). 5 μg of each ZFN-encoding plasmid (RNA) and 5 μg of the targeting plasmid (DNA digested with ClaI and NotI, gel purified) were used to transfect 1 million GCK^{G299R/+} cells. Transfection was performed using Amaxa Nucleofector (program A-13) and Human Stem Cell Solution I (Lonza). After transfection, cells were seeded on 10 cm culture dish and allowed to recover for 2 day. Cells were then selected by 2-days of exposure to hygromycin (50 ng/ml). Resistant colonies were screened by PCR. The GCK^{+/hygro} cells were transfected by 5 μg of the correction plasmid and 5 μg of a plasmid carrying the I-SceI enzyme using the method described above. After transfection, cells were treated with 2 μg/ml ganciclovir for 2 days. PCR and sequencing were used to screen the resistant colonies and Southern blotting was used to characterize the genomic structure.

Southern blotting was performed using the DIG System following manufacturer's instruction (Roche). Primers for probe synthesis are listed in Table 3. DNA from stem cells was prepared using High Pure PCR Template Preparation Kit (Roche). 10 μg of DNA from each cell line was digested with BglII and XbaI.

Immunostaining

Cultured cells were briefly washed with PBS and fixed with 4% paraformaldehyde for 30 minutes at room temperature. Embryoid bodies and mouse kidneys were fixed with 4% paraformaldehyde overnight at 4° C., dehydrated using 15% (w/v) sucrose and 30% (w/v) sucrose solution and embedded in OCT compound (Tissue-Tek) before frozen under −80° C. Prior to staining, cells or frozen sections were blocked in 5% normal donkey serum for 30 minutes. Primary antibodies used in the study were as follows: mouse-anti-C-peptide (05-1109; Millipore), goat-anti-glucagon (A056501; DAKO), goat-anti-PDX1 (AF2419; R&D systems), goat-anti-SOX17 (AF1924; R&D systems), mouse-anti-OCT4 (sc-5279; Santa Cruz Biotechnology), rabbit-anti-SOX2 (09-0024; Stemgent), mouse-anti-SSEA4 (MAB1435; R&D systems), goat-anti-NANOG (AF1997; R&D systems), mouse-anti-TRA1-60 (MAB4360; Millipore), rabbit-anti-AFT (A000829; DAKO), mouse-anti-NKX2.2 and mouse-anti-MF20 (DSHB), rabbit-anti-TUJ1 (T3952; Sigma), sheep-anti-NGN3 (SAB3300089; Sigma), rabbit-anti-Ki67 (ab15580, Abcam). Appropriate second antibodies were obtained from Invitrogen. Quantification of positively stained cells was performed using the Celigo Cytometer system (Cyntellect).

Transplantation

On day 12 of differentiation, cells were dissociated using trypLE (5 minutes at room temperature). Aliquots of 2-3 million cells were collected into an eppendorf tube. Cells were spun down and the supernatant discarded. 10-15 ul matrigel (BD Biosciences) was added into each tube. Each tube of cell mixture was transplanted under the kidney capsule of an immunodeficient mouse NOD.Cg-Prkdc^{scid} Il2rg^{tm1Wjl}/SzJ (005557; The Jackson Laboratory), which lacks mature T or B cells, NK cells and cytokine signaling (32), following a previously described protocol (33). Also, ˜300 human islets obtained from National Disease Research Interchange were transplanted into each immunodeficient mouse. Three month after transplantation, human c-peptide was detected in the serum of the recipient mouse. An intraperitoneal glucose tolerance test was performed between 100-120 days after transplantation.

Insulin Secretion Assay

Typically, cells were cultured in 12-well dishes. After 12 days of differentiation, cells were washed for 1 hour in CMRL medium. Cells were then incubated in 300 μl CMRL medium containing 5.6 mM glucose for 1 hour and the medium was collected. Subsequently, 300 μA CMRL medium containing 16.9 mM glucose or other secretagogues was used to treat cells for 1 hour, following which the medium was collected. For in vivo tests, mice were deprived of food overnight with ad libitium access to water. After 12-14 hours of fasting, capillary blood glucose concentrations were determined by tail vein bleed using an AlphaTRACK glucometer (Abbott). Venous blood samples were collected via the submandibular vein. Intraperitoneal glucose was then administered (1 mg/g body weight) and ½ hour later blood samples were obtained via the submandibular vein. Blood samples were kept at room temperature for 2 hours and serum was obtained by centrifuging blood samples at 4000 rpm for 15 min. C-peptide concentrations in medium or mouse serum were measure using an ultrasensitive human C-peptide ELISA kit according to manufacturer's instructions (Mercodia). All mouse studies were reviewed and approved by the institutional animal care and use committee (IACUC) of Columbia University.

Transmission Electron Microscopy

Cells were fixed with 2.5% glutaraldehyde in 0.1 M Sorenson's buffer (pH 7.2) for 1 hour. Further processing and imaging of the samples was performed by Diagnostic Service, Department of Pathology and Cell Biology, Columbia University. Insulin granules were defined as electron-dense granular structures using a magnification of ×7,500. The number of insulin granules was determined for 3 cells of each cell line by manual counting.

### REFERENCES

1. Park, I. H. et al. Disease-specific induced pluripotent stem cells. Cell 134, 877-886 (2008).
2. Maehr, R. et al. Generation of pluripotent stem cells from patients with type 1 diabetes. Proc Natl Acad Sci USA 106, 15768-15773 (2009).
3. Kroon, E. et al. Pancreatic endoderm derived from human embryonic stem cells generates glucose-responsive insulin-secreting cells in vivo. Nat Biotechnol 26, 443-452 (2008).
4. Fajans, S. S., Bell, G. I. & Polonsky, K. S. Molecular mechanisms and clinical pathophysiology of maturity-onset diabetes of the young. N Engl J Med 345, 971-980 (2001).
5. Dean, H. & McEntyre, J. The Genetic Landscape of Diabetes. National Center for Biotechnology Information (US) (2004).
6. Ehtisham, S., Hattersley, A. T., Dunger, D. B. & Barrett, T. G. First UK survey of paediatric type 2 diabetes and MODY. Arch Dis Child 89, 526-529 (2004).
7. Estalella, I. et al. Mutations in GCK and HNF-1alpha explain the majority of cases with clinical diagnosis of MODY in Spain. Clin Endocrinol (Oxf) 67, 538-546 (2007).
8. Byrne, M. M. et al. Insulin secretory abnormalities in subjects with hyperglycemia due to glucokinase mutations. J Clin Invest 93, 1120-1130 (1994).
9. Njolstad, P. R. et al. Permanent neonatal diabetes caused by glucokinase deficiency: inborn error of the glucose-insulin signaling pathway. Diabetes 52, 2854-2860 (2003).
10. Cuesta-Munoz, A. L. et al. Severe persistent hyperinsulinemic hypoglycemia due to a de novo glucokinase mutation. Diabetes 53, 2164-2168 (2004).
11. Froguel, P. et al. Familial hyperglycemia due to mutations in glucokinase. Definition of a subtype of diabetes mellitus. N Engl J Med 328, 697-702 (1993).
12. Terauchi, Y. et al. Pancreatic beta-cell-specific targeted disruption of glucokinase gene. Diabetes mellitus due to defective insulin secretion to glucose. J Biol Chem 270, 30253-30256 (1995).
13. Terauchi, Y. et al. Glucokinase and IRS-2 are required for compensatory beta cell hyperplasia in response to high-fat diet-induced insulin resistance. J Clin Invest 117, 246-257 (2007).
14. Fusaki, N., Ban, H., Nishiyama, A., Saeki, K. & Hasegawa, M. Efficient induction of transgene-free human pluripotent stem cells using a vector based on Sendai virus, an RNA virus that does not integrate into the host genome. Proc Jpn Acad Ser B Phys Biol Sci 85, 348-362 (2009).
15. Zhu, H., Lensch, M. W., Cahan, P. & Daley, G. Q. Investigating monogenic and complex diseases with pluripotent stem cells. Nat Rev Genet. 12, 266-275 (2011).
16. Zou, J. et al. Gene targeting of a disease-related gene in human induced pluripotent stem and embryonic stem cells. Cell Stem Cell 5, 97-110 (2009).
17. Hockemeyer, D. et al. Genetic engineering of human pluripotent cells using TALE nucleases. Nat Biotechnol 29, 731-734 (2011).
18. D'Amour, K. A. et al. Production of pancreatic hormone-expressing endocrine cells from human embryonic stem cells. Nat Biotechnol 24, 1392-1401 (2006).
19. Redmer, T. et al. E-cadherin is crucial for embryonic stem cell pluripotency and can replace OCT4 during somatic cell reprogramming EMBO Rep 12, 720-726 (2011).
20. Thiery, J. P. & Sleeman, J. P. Complex networks orchestrate epithelial-mesenchymal transitions. Nat Rev Mol Cell Biol 7, 131-142 (2006).
21. Alattia, J. R., Tong, K. I., Takeichi, M. & Ikura, M. Cadherins. Methods Mol Biol 172, 199-210 (2002).
22. Song, W. J. et al. Exendin-4 stimulation of cyclin A2 in beta-cell proliferation. Diabetes 57, 2371-2381 (2008).
23. Nostro, M. C. et al. Stage-specific signaling through TGFbeta family members and WNT regulates patterning and pancreatic specification of human pluripotent stem cells. Development 138, 861-871 (2011).
24. Furuya, F., Shimura, H., Yamashita, S., Endo, T. & Kobayashi, T. Liganded thyroid hormone receptor-alpha enhances proliferation of pancreatic beta-cells. J Biol Chem 285, 24477-24486 (2010).
25. Porat, S. et al. Control of pancreatic beta cell regeneration by glucose metabolism. Cell Metab 13, 440-449 (2011).
26. Turkkahraman, D. et al. Permanent neonatal diabetes mellitus caused by a novel homozygous (T168A) glucokinase (GCK) mutation: initial response to oral sulphonylurea therapy. J Pediatr 153, 122-126 (2008).
27. Sandhu, M. S. et al. Common variants in WFS1 confer risk of type 2 diabetes. Nat Genet 39, 951-953 (2007).
28. Scott, L. J. et al. A genome-wide association study of type 2 diabetes in Finns detects multiple susceptibility variants. Science 316, 1341-1345 (2007).
29. Dupuis, J. et al. New genetic loci implicated in fasting glucose homeostasis and their impact on type 2 diabetes risk. Nat Genet. 42, 105-116 (2010).
30. Vaxillaire, M. et al. Impact of common type 2 diabetes risk polymorphisms in the DESIR prospective study. Diabetes 57, 244-254 (2008).
31. Noggle, S. et al. Human oocytes reprogram somatic cells to a pluripotent state. Nature 478, 70-75 (2011).
32. Shultz, L. D. et al. Human lymphoid and myeloid cell development in NOD/LtSz-scid IL2R gamma null mice engrafted with mobilized human hemopoietic stem cells. J Immunol 174, 6477-6489 (2005).
33. Szot, G. L., Koudria, P. & Bluestone, J. A. Transplantation of pancreatic islets into the kidney capsule of diabetic mice. J Vis Exp, 404 (2007).

### Example 8

### A Stem Cell Model of Diabetes Due to Glucokinase Deficiency

Diabetes is a disorder characterized by loss of beta cell mass and/or beta cell function, leading to deficiency of insulin relative to metabolic need. To determine whether stem cell-derived beta cells recapitulate molecular-physiological phenotypes of a diabetic subject, induced pluripotent stem (iPS) cells were generated from diabetic subjects (MODY2) with heterozygous loss-of-function of the gene encoding glucokinase (GCK). These stem cells differentiated into beta cells with an efficiency comparable to controls, and expressed markers of mature beta cells, urocortin-3 and zinc transporter 8 upon transplantation into mice. While insulin secretion in response to arginine or other secretagogues was identical to cells from healthy controls, GCK mutant beta cells required higher glucose levels to stimulate insulin secretion. Importantly this glucose-specific phenotype was fully reverted upon gene sequence correction by homologous recombination. These results demonstrate that iPS cell-derived beta cells reflect beta cell-autonomous phenotypes of MODY2 subjects, providing a platform for mechanistic analysis of specific genotypes on beta cell function.

Recent progress in somatic cell reprogramming has allowed the generation of induced pluripotent stem cells (iPSCs) from diabetic subjects (A1). Human pluripotent stem cells, including iPS cells and human embryonic stem cells, have the capacity to differentiate into insulin-producing cells (A2), which display key properties of true beta cells, including glucose-stimulated insulin secretion upon maturation in vivo (A3). iPS cells have been generated from patients with various types of diabetes (A2, A4, A5). However, whether iPSC-derived beta cells can accurately replicate pathologic phenotypes, and be used to test strategies to restore normal beta cell function, is not clear. As proof-of-principle, a monogenic form of diabetes, MODY2, was modeled (A6).

Maturity-onset diabetes of the young (MODY) is caused by single gene mutations, resulting in defects in the development, proliferation/regeneration, and/or function of beta cells (A7). MODY accounts for 1 to 5 percent of all instances of diabetes in the United States (A8), and MODY2, caused by mutations in the glucokinase (GCK) gene, accounts for 8-60% of all MODY cases, depending on population sampling (A9, A10). Glucokinase links blood glucose levels to insulin secretion by converting glucose to glucose-6-phosphate, the rate-limiting step in glycolysis. The catalytic capacity of glucokinase in beta cells determines the threshold for glucose stimulated insulin secretion. Due to hypofunction of one allele of GCK, the dose-response curve relating glucose and insulin secretion obtained with graded glucose infusions is shifted to the right in the MODY2 subjects, resulting in mild hyperglycemia (A11). Subjects with permanent neonatal diabetes, caused by the absence of both GCK alleles, are insulin-dependent at birth and show intrauterine growth retardation (A12). In a mouse model, heterozygous loss of GCK causes hyperglycemia, early-onset diabetes (10 weeks old), reduced response to glucose stimulation (A13), and an inability to increase beta cell mass under conditions of insulin resistance (A14). Mouse islets with homozygous loss of GCK fail to increase insulin release in response to glucose in vitro (A13).

These well-characterized consequences in mice and humans allow assessment of the accuracy of stem cell models for diabetes. Such models will offer significant advantages over a genetically manipulated mouse or human subjects for preclinical testing of therapeutic strategies and for drug screening, and for studies designed to gain insight into the molecular mechanisms how specific genotypes affect beta cell function and cause diabetes in human subjects. For example, while it is known that GCK affects glucose-stimulated insulin secretion, whether insulin biosynthesis and/or beta cell proliferation is also affected could not be determined in human subjects.

It was found that induced pluripotent stem cells (iPSCs) from MODY2 subjects heterozygous for hypomorphic GCK mutations differentiated into insulin-producing beta cells with an efficiency comparable to controls. In contrast, stem cells with two inactive GCK alleles showed a reduced capacity to generate insulin-producing cells. Hypomorphic GCK alleles reduced insulin secretion specifically in response to glucose, but not in response to other secretagogues, including arginine. The responsiveness to glucose was restored when the GCK mutation was corrected by homologous recombination. These results demonstrate that iPSC-derived patient-specific beta cells recapitulate the anticipated functional phenotypes observed in human subjects, and enable analysis of aspects of cellular physiology not otherwise possible.

Stem Cells with an Allelic Series at the GCK Locus

Skin biopsies were obtained from two MODY2 subjects, a 38 year old Caucasian female diagnosed with diabetes at the age of 21 years and a 56 year old Caucasian male who was diagnosed with diabetes at age 47. Both of them had a family history of diabetes, were negative for antibodies associated with type 1 diabetes, non-obese (BMI=21 to 26 kg/m2) and positive for measurable, but low serum C-peptide (0.1 to 0.4 ng/ml) (FIG. 10). MODY2 subjects typically display mild fasting hyperglycemia and can generally be managed with dietary therapy alone, while additional pharmacotherapy is sometimes used to optimally control blood glucose excursions (A15). In the two MODY2 subjects from whom skin biopsies were obtained, diabetes control was excellent (HbAlC's≦6.5%) on insulin, or sulfonylurea-related agents (Table 1). Exonic sequencing of GCK revealed that the female subject carries a missense mutation (G299R), and the male subject a missense mutation (E256K) (FIG. 7A). Both mutations have been shown to be functionally hypomorphic, with less than 1% of activity of the wild type allele (A16).

Induced pluripotent stem cell lines were generated from skin cell lines using non-integrating Sendai viruses (FIGS. 11A-B) (A17). The iPS cells with the hypomorphic GCK mutations had the expression profile of pluripotent cells and the capability to differentiate into endodermal, mesodermal and ectodermal tissues (FIGS. 11C-E). Because of genetic diversity in humans, controlling for effects of the genetic background is critical for functional comparisons between mutant and non-mutant cells (A18). To generate cell lines with identical genetic background, but with different genotypes at the GCK locus, targeted genetic modifications were performed (FIG. 7B). A two-step targeting protocol was designed that allowed the precise correction of the mutant base pair without leaving a footprint of exogenous DNA. First, the GCK locus was targeted with a linearized construct containing a PGK-hygro-TK fusion gene, flanked by two segments of the GCK locus corresponding to intron 6 and exon 10 in GCK^{G299R/+} cells. Messenger RNA encoding a zinc finger nuclease to induce a double-strand break (DSB) 1150 bp upstream of the G299R mutation was introduced into GCK^{G299R/+} cells with the targeting plasmid to facilitate homologous recombination. Hygromycin-resistant colonies of transfected GCK^{G299R/+} cells were expanded and tested for homologous integration using PCR primers annealing to the genomic sequence and to the hygro-TK cassette (FIG. 7B). Of 201 hygromycin-resistant colonies, 14 (7%) showed targeting of the construct to either the wild type or the mutant allele, resulting in GCK^{G299R/hygro} and GCK^{+/hygro} cells, respectively (FIG. 7C). Cells carrying the GCK^{hygro} allele with exons 7 to 10 disrupted, in combination with the G299R mutation are expected to have very little, if any, GCK activity.

In a second step, two wild type copies of GCK were restored in GCK^{+/hygro} cells using a plasmid containing the wild type GCK locus, marked with a SNP in intron 7 to be able to distinguish the two copies of GCK. A plasmid encoding the endonuclease I-SceI site was co-transfected to induce a DSB at an I-SceI recognition site located in the hygro-TK cassette to facilitate homologous recombination and to replace all vector sequences, including the TK gene (FIG. 7B). Ganciclovir-resistant colonies were screened for homologous integration using PCR with one primer outside of the targeting construct and one primer within the construct, followed by sequencing of the induced SNP. 2 of 96 colonies (2% efficiency) had correctly targeted to the GCK locus and restored two wild type copies of GCK, which was also confirmed by Southern blotting (FIG. 7D); these cells were karyotypically normal (FIG. 7E), and designated GCK^{corrected/+}. These targeted manipulations resulted in an allelic series of cells that were wild type (GCK^{corrected/+}), hypomorphic (GCK^{G299R/+}) and or null (GCK^{G299R/hygro}) for GCK function on the same genetic background, allowing exclusion of potential confounding effects of different genetic backgrounds in subsequent experiments.

Efficient Beta Cell Generation from GCK Deficient iPS Cells

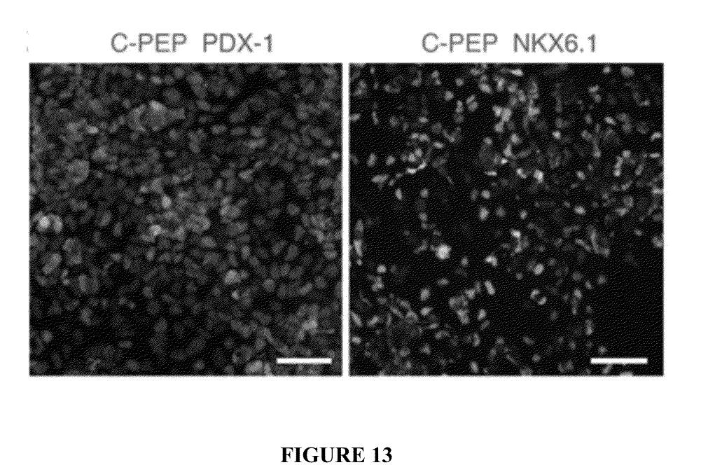

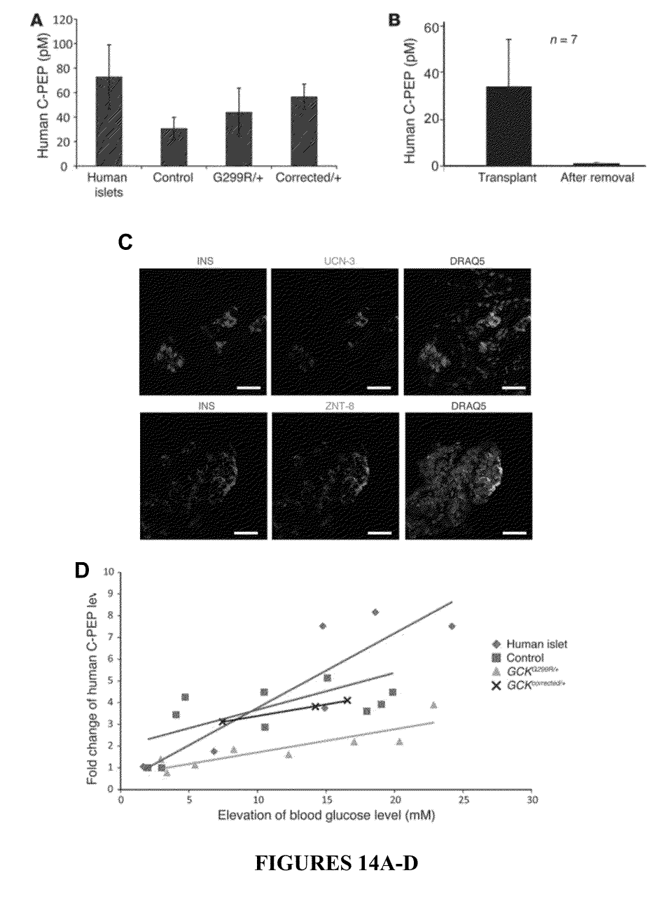

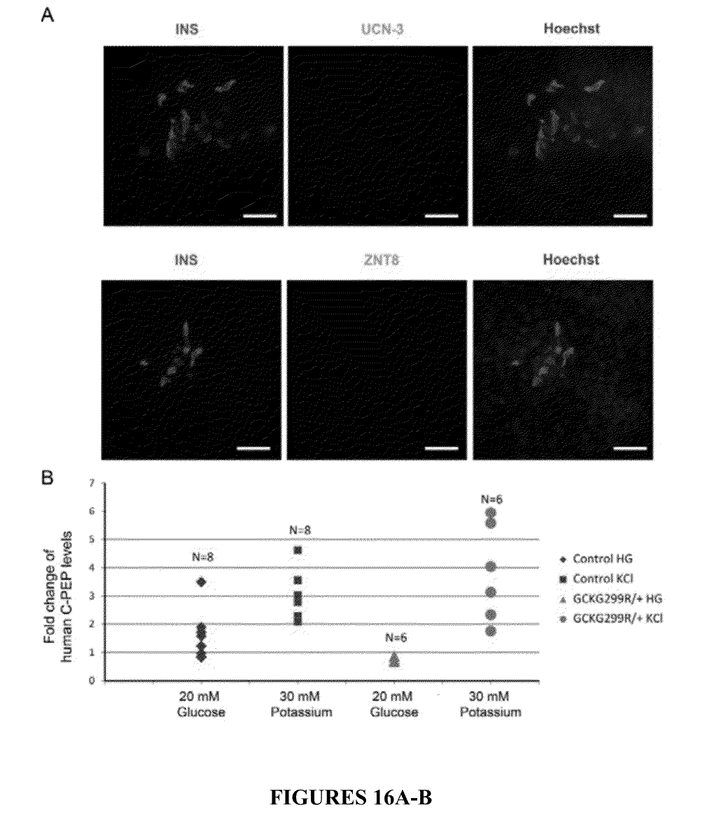

Human embryonic stem cells and iPS cells can be differentiated towards insulin producing cells after stepwise differentiation into definitive endoderm (SOX17+), pancreatic progenitors (PDX1+) and endocrine progenitors (NGN3+) (A2, A19). While published protocols yielded SOX17− and PDX1− positive cells, insulin-producing cells were not obtained (FIG. 12A). Three days after induction of differentiation (Stage 1) it was noticed that colonies with the morphology of pluripotent stem cells were still apparent. These cells retained Oct4 expression and failed to commit to the endoderm lineage, as evidenced by the lack of Sox17 expression (FIG. 12B). It was reasoned that interfering with the pluripotent state should increase the ability of Activin to direct differentiation towards the endoderm lineage. Cell-to-cell interactions mediated by E-cadherin are critical for maintaining pluripotency of ES cells (A20). When the calcium chelator EGTA, an inhibitor or cadherin-mediated cell-cell attachment, was added on the first day of differentiation, the tight colony morphology of iPS cells was lost (FIG. 8A). In parallel, the percentage of OCT4+SOX17− cells was reduced from 5% to 2% (FIG. 8B), while the percentage of endodermal (SOX17+OCT4−) cells was increased by 25.5% (FIG. 8C). These responses, in the aggregate, resulted in a 21.7% (mean of 4 different cell lines) increase in PDX1 positive cells on day 8 of differentiation (Stage 3) (FIG. 8D). To further improve differentiation conditions from pancreatic progenitor to beta cells, exendin-4, a glucagon-like peptide-1 agonist, and SB431542, a TGFbeta signaling inhibitor, were added to Stage 3 progenitor cells. Both of these additions enhanced the differentiation efficiency of beta cells to 4.6% and 8.2% (C-PEP+), respectively, consistent with previous observations (A21-A23). A combination of exendin-4 and SB431542 treatment from day 9 to day 12 produced the highest percentage of beta cells (15%) (FIG. 8E). It was found that our modified protocol efficiently induced differentiation of both ES and iPS cells (FIG. 8E; FIG. 9E; FIG. 12F). When differentiated into beta cells, control and MODY2 stem cells showed similar efficiency of generating C-PEP+ cells from PDX1+ progenitors (FIG. 12F). These cells expressed beta cell transcriptional factor PDX-1 and NKX6.1 (FIG. 13). To assess the temporal expression pattern of GCK during the in vitro differentiation process, GCK mRNA levels were measured at definitive endoderm (day 3 of differentiation), pancreatic endoderm (day 8) and endocrine (day 12) stages. Expression of GCK was detected only at the endocrine stage, coinciding with the expression of insulin (FIG. 8F). It was observed that in the differentiation culture 38% of the insulin-producing cells also immunostained for glucagon and 14% of the insulin-producing cells also expressed somatostatin, similar to previous observations (A19) (FIG. 12C). Cells co-producing insulin and glucagon also appear during development of human fetal pancreas (A24), suggesting that these cells were not fully differentiated. Further differentiation into monohormonal cells occurred in vivo, after transplantation of cells at day 12 of differentiation under the kidney capsule of immune-compromised mice. Three months after transplantation, 24 of 50 mice had detectable human C-peptide in their serum (FIG. 14A). To determine whether the C-peptide originated from the transplants, we removed the transplants from 7 mice, and found that none retained detectable human C-peptide in the serum (FIG. 14B) Immunohistochemistry of the isolated graft showed that hormone-expressing cells in the transplants expressed solely insulin, glucagon or somatostatin (FIG. 8G). It was also observed the presence of urocortin-3 and zinc transporter 8 in the insulin-positive cells in the transplants (FIG. 14C), while these markers of mature beta cells were absent in beta cells derived in vitro (FIG. 16A) (A25, A26).

GCK Mutations Specifically Affect Glucose Mediated Insulin Secretion

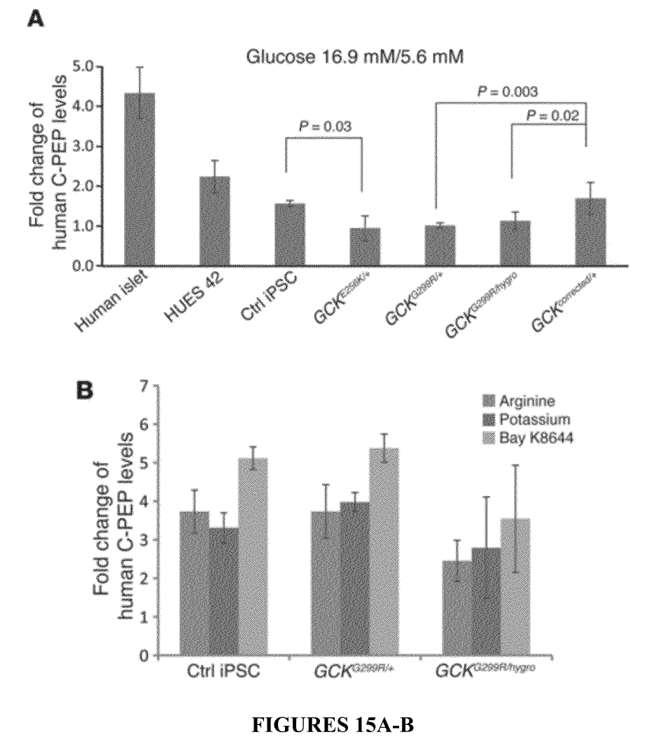

Beta cells in MODY2 patients with GCK mutations are able to respond to glucose but with reduced sensitivity (A11). To determine if this phenotype can be recapitulated by iPSC-derived beta cells, intraperitoneal glucose tolerance tests were performed on transplanted mice. Both human C-peptide and glucose concentrations were measured in the blood and a dose-responsiveness of c-peptide to circulating blood glucose concentration was found. The sensitivity of human insulin-producing transplanted cells were evaluated by assessing the slopes of these relationships. GCK^{G299R/+} cells showed a reduced sensitivity to glucose compared to control cells (FIG. 14D). Gene correction in GCK^{corrected/+} cells restored glucose sensitivity to that of control cells. If GCK effects are mediated solely by impact on glucose sensing, insulin secretion in response to secretagogues acting independently of glycolysis should be unaffected. To test this possibility, first glucose-stimulated insulin secretion (GSIS) assays were performed on in vitro differentiated beta cells. In order to bracket physiologically-relevant concentrations of glucose, iPSC-derived beta cells and human islets were treated with 5.6 mM and 16.9 mM. 2.5 mM and 20 mM glucose were also used to treat control and MODY2 iPSC-derived beta cells. Beta cells derived from human ES cells and control iPS cells showed increased in C-peptide secretion (mean: 2.1 fold, range 0.8-3.5; 21 of 28 biological replicates showed >1.2 fold increase). In contrast GCK^{E256K/+}, GCK^{G299R/+} and GCK^{G299R/hygro} cells showed no increase (mean: 0.9, fold, range 0.7-1.1; none of the 25 biological replicas showed >1.2 fold increase) (FIG. 15A; FIG. 16B). Importantly, correction of the G299R mutation to the wild type nucleotide sequence, restored glucose responsiveness: GCK^{corrected/+} cells showed a 1.6-fold increase in glucose-stimulated C-peptide secretion (range: 1.1-2.3; 4 of 5 biological replicas showed >1.2 fold increase, P=0.003) (FIG. 15A). When exposed to other secretagogues, GCK^{G299R/+} and GCK^{G299R/hygro} cells increased C-peptide release in response to arginine (3-4 fold), potassium (3-4 fold), and to Bay K8644, a calcium channel agonist (3-5 fold), identical to control cells (FIG. 15B; FIG. 16B). Therefore, GCK mutations specifically affect glucose-mediated insulin secretion.

GCK mutations may also affect other aspects of beta cell function, such as production/processing of insulin precusors, or by interfering with insulin secretion or beta cell proliferation. These different possibilities have thus far not been addressed in human cells. It was found that insulin content was comparable in control beta cells and cells with genotype of GCK^{G299R/+}, GCK^{G299R/hygro} and GCK^{corrected/+} (FIG. 9A). By electron microscopy, cellular granule morphology and numbers were comparable in wild type (average 173 granules per cross-section) and GCK^{G299R/+} (average 220 granules per cross-section) (FIGS. 12D-E). It was also found that heterozygous loss of GCK didn't alter the yield of beta cells from PDX1+ progenitors, but a reduction in beta cell generation was observed in GCK^{G299R/hygro} cells (5% C-peptide positive versus 10% in GCK^{G299R/+} and GCK^{e}

(FIG. 9E). This difference could be caused by reduced replication of beta cells, because a reduction of Ki67 positive beta cells was observed (31% of C-peptide positive cells) in the GCK^{G299R/hygro} genotype, compared to the genotypes GCK^{G299R/+} (41%), GCK^{corrected/+} (45%) and control GCK^{+/+} (HUES42, 49%) (FIG. 9F). Therefore, haploinsufficiency of GCK does not affect insulin biosynthesis and proliferation of iPSC-derived beta cells in vitro.

Discussion

In this study, the fidelity with which beta cell-autonomous defects in a monogenic form of diabetes are reflected by iPSC-derived insulin-producing cells was tested. It was found that MODY2 beta cells responded to elevated glucose with lower sensitivity compared to gene-corrected control cells, but were otherwise comparable to control cells in insulin production and processing, and insulin secretion in response to other secretagogues, such as arginine. These findings demonstrate that cells heterozygous for hypomorphic GCK mutations recapitulate key aspects of the MODY2 phenotype.

The observation of anticipated phenotypes using iPSC-derived beta cells suggests that differences between GCK mutant and control cells that cannot readily be investigated in human cells may also reflect aspects of the human disease. It was found that in vitro differentiated beta cells carrying two inactive GCK alleles, but not cells with one or two functional GCK alleles, yielded a lower number of beta cells, at least partially by effects on proliferation. Though the beta cells generated in vitro show high rates of proliferation that are more similar those of the embryonic than the adult pancreas (A27), GCK is expressed in beta cells of fetal islets (A28, A29), and Porat et al. recently demonstrated a role for GCK in regulating beta cell proliferation in adult mice (A30). It was also found that in vitro, but not upon further differentiation in vivo, GCK mutant beta cells failed to increase insulin secretion at high ambient glucose concentrations. Whether this difference reflects an involvement of GCK in establishing responsiveness to glucose during functional maturation of beta cells remains to be investigated.

iPSC-derived cells can enable novel insights into the molecular-cell biology of beta cell failure in virtually all forms of diabetes. Stem cell models of diabetes should not only allow deeper insight into the consequences of specific mutations on beta cell function, but in doing so, also shed light on the molecular physiology of the beta cell in prevalent clinical circumstances. Common variants of WFS1 (A31), KCNJ11 (A32), and GCK (A33, A34) increase the risk of T2D diabetes. Stem cell-based approaches may also allow the investigation of genes modifying penetrance of specific mutations that affect beta cell function. Importantly, it was also possible to demonstrate that the specific correction of the mutant base pair in the GCK locus by homologous recombination restores glucose-stimulated insulin secretion. This system of homologous recombination offers a significant advantage over previously reported techniques, as it is both efficient, and does not result in the introduction of exogenous DNA sequences, such as loxP sites (A35). The generation of autologous beta cells in combination with gene correction may ultimately be useful for cell replacement to restore normal glucose homeostasis.

Methods

Research Subjects and Cell Lines

Biopsies of upper arm skin were obtained from two MODY2 subjects and a healthy subject using local anesthesia (lidocaine) and an AcuPunch biopsy kit (Acuderm Inc). Samples were coded and transported to the laboratory. Biopsies were cut in 10-12 small pieces, and 2-3 pieces of minced skin were placed around a silicon droplet in a well of six-well dish. A glass cover slip was placed over the biopsy pieces and 5 ml of biopsy plating media were added. After 5 days, biopsy pieces were grown in culture medium for 3-4 weeks. Biopsy plating medium was composed of DMEM, FBS, GlutaMAX, Anti-Anti, NEAA, 2-Mercaptoethanol and nucleosides (all from Invitrogen) and culture medium contained DMEM, FBS, GlutMAX and Pen-Strep (all from Invitrogen). HUES42 was chosen from a collection of Harvard University embryonic stem cell lines based on its robust and consistent ability to produce beta cells in vitro (A36).

Generation of Induced Pluripotent Stem Cells

Primary fibroblasts were converted into pluripotent stem cells using CytoTune™-iPS Sendai Reprogramming Kit (Invitrogen). 50,000 fibroblast cells were seeded per well of a six-well dish at passage three and allowed to recover overnight. Next day, Sendai viruses expressing human transcription factors Oct4, Sox2, Klf4 and C-Myc were mixed in fibroblast medium to infect fibroblast cells according to the manufacturer's instructions. Two days later, the medium was exchanged to human ES medium supplemented with the ALK5 inhibitor SB431542 (2 μM; Stemgent), the MEK inhibitor PD0325901 (0.5 μM; Stemgent), and thiazovivin (0.5 μM; Stemgent). Human ES medium contained KO-DMEM, KSR, GlutMAX, NEAA, 2-Mercaptoethanol, PenStrep and bFGF (all from Invitrogen). On day 7-10 post infection, cells were detached using TrypLE and passaged onto feeder cells. Individual colonies of induced pluripotent stem cells were picked between days 21-28 post infection and each iPS cell line was expanded from a single colony. All iPS cells lines were cultured on mouse embryonic fibroblast cells with human ES medium. Karyotyping was performed by Cell Line Genetics Inc. For teratoma analysis, 1-2 million cells from each iPS cell line were detached and collected after TrypLE (Invitrogen) treatment. Cells were suspended in 0.5 ml of human ES media. The cell suspension was mixed with 0.5 ml matrigel (BD Biosciences) and injected subcutaneously into dorsal flanks of an immunodeficient mouse (NOD.Cg-Prkdc^{scid} Il2rg^{tm1Wjl}/SzJ, Stock No.:005557, The Jackson Laboratory) (A37). 8-12 weeks after injection, teratomas were harvested, fixed overnight with 4% paraformaldehyde and processed according to standard procedures for paraffin embedding. The samples were then sectioned and HE (hematoxylin and eosin) stained.

Gene Expression Analysis

Total RNA was isolated from cells with RNAeasy kit (Qiagen). For quantitative PCR analysis, cDNA was synthesized using Promega RT system (Promega). Primers for qRT-PCR were listed in Table 3. For microarray analysis, RNA was prepared using Illumina Total Prep RNA amplification kit (Ambion) and hybridized to HumanRef-8 v3 Beadchip kit (Illumina). The global expression profiles of the samples were analyzed with normalization to average and subtraction of background using GenomeStudio Software (Illumina) and a hierarchical cluster tree was generated based on the correlation coefficients between samples. All array data are available on Gene Expression Omnibus under accession number GSE45777.

Directed Differentiation into Beta Cells

ES or iPS cells were dissociated using Dispase (3-5 min) and, subsequently, Accutase (5 min) Cells were suspended in human ES medium containing 10 uM ROCK inhibitor (Y27632) and filtered through 70 um cell strainer. Cells were then plated at a density of 400,000 cell/well in 12-well plates. After 1 or 2 days, when cells reached 80-90% confluency, differentiation was started. Detailed formulations of differentiation medium are listed in Table 4. Typically, cells were assayed between day 12 and day 16. For measuring proliferation rate, cells were assayed at day 12. Insulin contents were measured using Insulin ELISA kit (Mercodia).

Gene Targeting

A targeting vector (pBS-PGK-hytk-IsceI-LoxP) was constructed by cloning a PGK-hygro-TK cassette into a pBlueScript SK+ vector. A LoxP site was added upstream of the cassette. A LoxP and an I-SceI site were cloned downstream of the cassette. Two DNA fragments, “homologous arms”, from the glucokinase (GCK) gene (see Table 3 for primer sequences) were cloned into the pBS-PGK-hytk-IsceI-LoxP vector at 5′ and 3′ end of the cassette. A correction construct was created by cloning a DNA fragment of GCK (see Table 3 for primer sequences) into pCR2.1-TOPO vector using TOPO TA cloning kit (Invitrogen).

A pair of zinc-finger nucleases (ZFN) was designed by Sigma to recognize the following sequence in intron 7 of GCK: CGTCAATACCGAGTGgg-cgcCTTCGGGGACTCCGGC (UPPERCASE: ZFN-binding site, lowercase: cut site) (SEQ ID NO: 27). 5 μg of each ZFN-encoding plasmid (RNA) and 5 μg of the targeting plasmid (DNA digested with ClaI and NotI, gel purified) were used to transfect 1 million GCK^{G299R/+} cells. Transfection was performed using Amaxa Nucleofector (program A-13) and Human Stem Cell Solution I (Lonza). After transfection, cells were seeded on a 10 cm culture dish and allowed to recover for 2 day. Cells were then selected by 2-days of exposure to hygromycin (50 μg/ml). Resistant colonies were screened by PCR. The GCK^{+/hygro} cells were transfected by 5 μg of the correction plasmid and 5 μg of a plasmid carrying the I-SceI enzyme using the method described above. After transfection, cells were treated with 2 μg/ml ganciclovir for 2 days. PCR and sequencing were used to screen the resistant colonies and Southern blotting was used to confirm targeted integration. Southern blotting was performed using the DIG System following manufacturer's instruction (Roche). Primers for probe synthesis are listed in Table 3. DNA from stem cells was prepared using High Pure PCR Template Preparation Kit (Roche). 10 μg of DNA from each cell line was digested with BglII and XbaI.

Immunostaining

Cultured cells were briefly washed with PBS and fixed with 4% paraformaldehyde for 30 minutes at room temperature. Embryoid bodies and mouse kidneys were fixed with 4% paraformaldehyde overnight at 4° C., dehydrated using 15% (w/v) sucrose and 30% (w/v) sucrose solution, embedded in OCT compound (Tissue-Tek) and frozen at −80° C. Fixed cells or frozen sections were blocked in 5% normal donkey serum for 30 minutes. Primary antibodies used in the study were as follows: mouse-anti-C-peptide (05-1109; Millipore), goat-anti-glucagon (A056501; DAKO), goat-anti-PDX1 (AF2419; R&D systems), goat-anti-SOX17 (AF1924; R&D systems), mouse-anti-OCT4 (sc-5279; Santa Cruz Biotechnology), rabbit-anti-SOX2 (09-0024; Stemgent), mouse-anti-SSEA4 (MAB1435; R&D systems), goat-anti-NANOG (AF1997; R&D systems), mouse-anti-TRA1-60 (MAB4360; Millipore), rabbit-anti-AFT (A000829; DAKO), mouse-anti-NKX2.2 and mouse-anti-MF20 (DSHB), rabbit-anti-TUJ1 (T3952; Sigma), sheep-anti-NGN3 (SAB3300089; Sigma), rabbit-anti-Ki67 (ab15580, Abcam), rabbit-anti-UCN-3 (HPA038281, sigma), rabbit-anti-ZNT8 (Thermo Scientific, PA5-21010). Appropriate second antibodies were obtained from Invitrogen. Quantification of positively stained cells was performed using the Celigo Cytometer system (Cyntellect).

Transplantation

On day 12 of differentiation, cells were dissociated using trypLE (5 minutes at room temperature). Aliquots of 2-3 million cells were collected into an eppendorf tube. Cells were spun down and the supernatant discarded. 10-15 ul matrigel (BD Biosciences) was added into each tube. Each tube of cell mixture was transplanted under the kidney capsule of an immunodeficient mouse NOD.Cg-Prkdc^{scid} Il2rg^{tm1Wjl}/SzJ (005557; The Jackson Laboratory) (A37), following a previously described protocol (A38). For human islet transplantation ˜300 human islets obtained from National Disease Research Interchange were transplanted. Three months after transplantation, human c-peptide was determined in the serum of recipient mice. An intraperitoneal glucose tolerance test was performed between 100-120 days after transplantation.

Insulin Secretion Assay

Typically, cells were cultured in 12-well dishes. After 12 days of differentiation, cells were washed for 1 hour in CMRL medium. Cells were then incubated in 300 μl CMRL medium containing 5.6 mM glucose for 1 hour and the medium was collected. Subsequently, 300 μl CMRL medium containing 16.9 mM glucose or other secretagogues was used to treat cells for 1 hour, following which the medium was collected. For in vivo tests, mice were deprived of food overnight with ad libitum access to water. After 12-14 hours of fasting, capillary blood glucose concentrations were determined by tail bleed using an AlphaTRACK glucometer (Abbott). Venous blood samples were collected via the submandibular vein. Intraperitoneal glucose was then administered (1 mg/g body weight) and ½ hour later blood samples were obtained via the submandibular vein. Blood samples were kept at room temperature for 2 hours and serum was obtained by centrifuging blood samples at 4000 rpm for 15 min.

C-peptide concentrations in medium or mouse serum were measure using an ultrasensitive human C-peptide ELISA kit according to manufacturer's instructions (Mercodia). All mouse studies were reviewed and approved by the institutional animal care and use committee (IACUC) of Columbia University.

Transmission Electron Microscopy

Cells were fixed with 2.5% glutaraldehyde in 0.1 M Sorenson's buffer (pH 7.2) for 1 hour. Further processing and imaging of the samples was performed by Diagnostic Service, Department of Pathology and Cell Biology, Columbia University. Insulin granules were defined as electron-dense granular structures using a magnification of ×7,500. The number of insulin granules was determined for 3 cells of each cell line by manual counting.

Statistics

2 tailed Student's t test was used to determine statistical significance of differences between 2 groups. P values less than 0.05 were considered significant.

### REFERENCES

A1. Park, I. H., Arora, N., Huo, H., Maherali, N., Ahfeldt, T., Shimamura, A., Lensch, M. W., Cowan, C., Hochedlinger, K., and Daley, G. Q. 2008. Disease-specific induced pluripotent stem cells. Cell 134:877-886.
A2. Maehr, R., Chen, S., Snitow, M., Ludwig, T., Yagasaki, L., Goland, R., Leibel, R. L., and Melton, D. A. 2009. Generation of pluripotent stem cells from patients with type 1 diabetes. Proc Natl Acad Sci USA 106:15768-15773.
A3. Kroon, E., Martinson, L. A., Kadoya, K., Bang, A. G., Kelly, O. G., Eliazer, S., Young, H., Richardson, M., Smart, N. G., Cunningham, J., et al. 2008. Pancreatic endoderm derived from human embryonic stem cells generates glucose-responsive insulin-secreting cells in vivo. Nat Biotechnol 26:443-452.
A4. Ohmine, S., Squillace, K. A., Hartjes, K. A., Deeds, M. C., Armstrong, A. S., Thatava, T., Sakuma, T., Terzic, A., Kudva, Y., and Ikeda, Y. 2012. Reprogrammed keratinocytes from elderly type 2 diabetes patients suppress senescence genes to acquire induced pluripotency. Aging (Albany N.Y.) 4:60-73.
A5. Teo, A. K., Windmueller, R., Johansson, B. B., Dirice, E., Njolstad, P. R., Tjora, E., Raeder, H., and Kulkarni, R. N. 2013. Derivation of Human Induced Pluripotent Stem Cells from Patients with Maturity Onset Diabetes of the Young. J Biol. Chem.
A6. Velho, G., Froguel, P., Clement, K., Pueyo, M. E., Rakotoambinina, B., Zouali, H., Passa, P., Cohen, D., and Robert, J. J. 1992. Primary pancreatic beta-cell secretory defect caused by mutations in glucokinase gene in kindreds of maturity onset diabetes of the young. Lancet 340:444-448.
A7. Fajans, S. S., Bell, G. I., and Polonsky, K. S. 2001. Molecular mechanisms and clinical pathophysiology of maturity-onset diabetes of the young. N Engl J Med 345:971-980.
A8. Dean, H., and McEntyre, J. 2004. The Genetic Landscape of Diabetes. National Center for Biotechnology Information (US).
A9. Ehtisham, S., Hattersley, A. T., Dunger, D. B., and Barrett, T. G. 2004. First UK survey of paediatric type 2 diabetes and MODY. Arch Dis Child 89:526-529.
A10. Estalella, I., Rica, I., Perez de Nanclares, G., Bilbao, J. R., Vazquez, J. A., San Pedro, J. I., Busturia, M. A., and Castano, L. 2007. Mutations in GCK and HNF-1alpha explain the majority of cases with clinical diagnosis of MODY in Spain. Clin Endocrinol (Oxf) 67:538-546.
A11. Byrne, M. M., Sturis, J., Clement, K., Vionnet, N., Pueyo, M. E., Stoffel, M., Takeda, J., Passa, P., Cohen, D., Bell, G. I., et al. 1994. Insulin secretory abnormalities in subjects with hyperglycemia due to glucokinase mutations. J Clin Invest 93:1120-1130.
A12. Njolstad, P. R., Sagen, J. V., Bjorkhaug, L., Odili, S., Shehadeh, N., Bakry, D., Sarici, S. U., Alpay, F., Molnes, J., Molven, A., et al. 2003. Permanent neonatal diabetes caused by glucokinase deficiency: inborn error of the glucose-insulin signaling pathway. Diabetes 52:2854-2860.
A13. Terauchi, Y., Sakura, H., Yasuda, K., Iwamoto, K., Takahashi, N., Ito, K., Kasai, H., Suzuki, H., Ueda, O., Kamada, N., et al. 1995. Pancreatic beta-cell-specific targeted disruption of glucokinase gene. Diabetes mellitus due to defective insulin secretion to glucose. J Biol Chem 270:30253-30256.
A14. Terauchi, Y., Takamoto, I., Kubota, N., Matsui, J., Suzuki, R., Komeda, K., Hara, A., Toyoda, Y., Miwa, I., Aizawa, S., et al. 2007. Glucokinase and IRS-2 are required for compensatory beta cell hyperplasia in response to high-fat diet-induced insulin resistance. J Clin Invest 117:246-257.
A15. Naylor, R., and Philipson, L. H. 2011. Who should have genetic testing for maturity-onset diabetes of the young? Clin Endocrinol (Oxf) 75:422-426.
A16. Gidh-Jain, M., Takeda, J., Xu, L. Z., Lange, A. J., Vionnet, N., Stoffel, M., Froguel, P., Velho, G., Sun, F., Cohen, D., et al. 1993. Glucokinase mutations associated with non-insulin-dependent (type 2) diabetes mellitus have decreased enzymatic activity: implications for structure/function relationships. Proc Natl Acad Sci USA 90:1932-1936.
A17. Fusaki, N., Ban, H., Nishiyama, A., Saeki, K., and Hasegawa, M. 2009. Efficient induction of transgene-free human pluripotent stem cells using a vector based on Sendai virus, an RNA virus that does not integrate into the host genome. Proc Jpn Acad Ser B Phys Biol Sci 85:348-362.
A18. Zhu, H., Lensch, M. W., Cahan, P., and Daley, G. Q. 2011. Investigating monogenic and complex diseases with pluripotent stem cells. Nat Rev Genet. 12:266-275.
A19. D'Amour, K. A., Bang, A. G., Eliazer, S., Kelly, O. G., Agulnick, A. D., Smart, N. G., Moorman, M. A., Kroon, E., Carpenter, M. K., and Baetge, E. E. 2006. Production of pancreatic hormone-expressing endocrine cells from human embryonic stem cells. Nat Biotechnol 24:1392-1401.
A20. Redmer, T., Diecke, S., Grigoryan, T., Quiroga-Negreira, A., Birchmeier, W., and Besser, D. 2011. E-cadherin is crucial for embryonic stem cell pluripotency and can replace OCT4 during somatic cell reprogramming. EMBO Rep 12:720-726.
A21. Song, W. J., Schreiber, W. E., Zhong, E., Liu, F. F., Kornfeld, B. D., Wondisford, F. E., and Hussain, M. A. 2008. Exendin-4 stimulation of cyclin A2 in beta-cell proliferation. Diabetes 57:2371-2381.
A22. Nostro, M. C., Sarangi, F., Ogawa, S., Holtzinger, A., Corneo, B., Li, X., Micallef, S. J., Park, I. H., Basford, C., Wheeler, M. B., et al. 2011. Stage-specific signaling through TGFbeta family members and WNT regulates patterning and pancreatic specification of human pluripotent stem cells. Development 138:861-871.
A23. Furuya, F., Shimura, H., Yamashita, S., Endo, T., and Kobayashi, T. 2010. Liganded thyroid hormone receptor-alpha enhances proliferation of pancreatic beta-cells. J Biol Chem 285:24477-24486.
A24. De Krijger, R. R., Aanstoot, H. J., Kranenburg, G., Reinhard, M., Visser, W. J., and Bruining, G. J. 1992. The midgestational human fetal pancreas contains cells coexpressing islet hormones. Dev Biol 153:368-375.
A25. Blum, B., Hrvatin, S. S., Schuetz, C., Bonal, C., Rezania, A., and Melton, D. A. 2012. Functional beta-cell maturation is marked by an increased glucose threshold and by expression of urocortin 3. Nat Biotechnol 30:261-264.
A26. Chimienti, F., Favier, A., and Seve, M. 2005. ZnT-8, a pancreatic beta-cell-specific zinc transporter. Biometals 18:313-317.
A27. Kassem, S. A., Ariel, I., Thornton, P. S., Scheimberg, I., and Glaser, B. 2000. Beta-cell proliferation and apoptosis in the developing normal human pancreas and in hyperinsulinism of infancy. Diabetes 49:1325-1333.
A28. Mally, M. I., Otonkoski, T., Lopez, A. D., and Hayek, A. 1994. Developmental gene expression in the human fetal pancreas. Pediatr Res 36:537-544.
A29. Tu, J., Tuch, B. E., and Si, Z. 1999. Expression and regulation of glucokinase in rat islet beta- and alpha-cells during development. Endocrinology 140:3762-3766.
A30. Porat, S., Weinberg-Corem, N., Tornovsky-Babaey, S., Schyr-Ben-Haroush, R., Hija, A., Stolovich-Rain, M., Dadon, D., Granot, Z., Ben-Hur, V., White, P., et al. 2011. Control of pancreatic beta cell regeneration by glucose metabolism. Cell Metab 13:440-449.
A31. Sandhu, M. S., Weedon, M. N., Fawcett, K. A., Wasson, J., Debenham, S. L., Daly, A., Lango, H., Frayling, T. M., Neumann, R. J., Sherva, R., et al. 2007. Common variants in WFS1 confer risk of type 2 diabetes. Nat Genet. 39:951-953.
A32. Scott, L. J., Mohlke, K. L., Bonnycastle, L. L., Willer, C. J., Li, Y., Duren, W. L., Erdos, M. R., Stringham, H. M., Chines, P. S., Jackson, A. U., et al. 2007. A genome-wide association study of type 2 diabetes in Finns detects multiple susceptibility variants. Science 316:1341-1345.
A33. Dupuis, J., Langenberg, C., Prokopenko, I., Saxena, R., Soranzo, N., Jackson, A. U., Wheeler, E., Glazer, N. L., Bouatia-Naji, N., Gloyn, A. L., et al. 2010. New genetic loci implicated in fasting glucose homeostasis and their impact on type 2 diabetes risk. Nat Genet. 42:105-116.
A34. Vaxillaire, M., Veslot, J., Dina, C., Proenca, C., Cauchi, S., Charpentier, G., Tichet, J., Fumeron, F., Marre, M., Meyre, D., et al. 2008. Impact of common type 2 diabetes risk polymorphisms in the DESIR prospective study. Diabetes 57:244-254.
A35. Liu, G. H., Suzuki, K., Qu, J., Sancho-Martinez, I., Yi, F., Li, M., Kumar, S., Nivet, E., Kim, J., Soligalla, R. D., et al. 2011. Targeted gene correction of laminopathy-associated LMNA mutations in patient-specific iPSCs. Cell Stem Cell 8:688-694.
A36. Chen, A. E., Egli, D., Niakan, K., Deng, J., Akutsu, H., Yamaki, M., Cowan, C., Fitz-Gerald, C., Zhang, K., Melton, D. A., et al. 2009. Optimal timing of inner cell mass isolation increases the efficiency of human embryonic stem cell derivation and allows generation of sibling cell lines. Cell Stem Cell 4:103-106.
A37. Shultz, L. D., Lyons, B. L., Burzenski, L. M., Gott, B., Chen, X., Chaleff, S., Kotb, M., Gillies, S. D., King, M., Mangada, J., et al. 2005. Human lymphoid and myeloid cell development in NOD/LtSz-scid IL2R gamma null mice engrafted with mobilized human hemopoietic stem cells. J Immunol 174:6477-6489.
A38. Szot, G. L., Koudria, P., and Bluestone, J. A. 2007. Transplantation of pancreatic islets into the kidney capsule of diabetic mice. J Vis Exp:404.

### Example 9

### Culture Mediums

Third culture medium, alternative: “wherein the third culture medium further comprises human KGF and FBS in RPMI medium”. Explanation: KGF is a replacement for FGF10; KAAD-cyclopamine is omitted.

Fourth culture medium, alternative: “wherein the fourth culture medium further comprises KAAD-cyclopamine, 4-[(E)-2-(5,6,7,8-Tetrahydro-5,5,8,8-tetramethyl-2-naphthalenyl)-1-propenyl]benzoic acid (TTNPB), LDN-193189, Activin A and 1×B27”.

Explanation: TTNPB is a replacement for retinoic acid; FGF 10 is omitted; Activin A is a new addition.

Fifth culture medium, alternative: “wherein the fifth culture medium is a DMEM high glucose medium comprising 1× Pen-Strep and 1× Glutamax and wherein the fifth culture medium further comprises exendin-4, ALK5 inhibitor and 1×B27”. Explanation: DMEM high glucose medium replaces CMRL medium; ALK5 inhibitor is a replacement for SB431542.

### Example 10

### Patient-Specific Beta Cells Reveals Phenotypes Due to HNF1A Haploinsufficiency

Transcription factors control beta cell differentiation, replication and function. The majority of instances of congenital forms of diabetes are caused by haploinsufficiency of transcription factors (e.g. HNF1A, HNF4A, HNF1B, PDX1). These genes, for instance HNF1A and HNF4A, have been linked to type 2 diabetes in genome wide association study. Systematically characterizing cellular and molecular defects in pancreatic beta cells deficient of these transcription factors will shed light on the mechanisms that underlie beta cell development, function and survival, and hence point to molecules implicated in the development and progression of diabetes. Stem cells were generated from diabetic subjects with heterozygous loss-of-function mutations of the gene, HNF1A which accounts for cases of MODY. Using stem cellderived patient-specific beta cells, cells with HNF1A mutations have reduced insulin-production and glucose response. Global transcriptional analysis indicates that expression of genes involved in glycolysis are decreased. Using a novel 3D culture system, the long term functionality of HNF1A mutant cells were decreased. Results reveal that deficiency of HNF1A has broad range of effects that lead to multiple functional consequences.

Sequence variations in hepatocyte nuclear factors (HNF1A and HNF4A) have been associated with type 2 diabetes (T2D) in [specify types of studies]^{1,2}. MODY3 is due to dominant hypomorphic mutations in HNF1A. Beta cells of MODY3 subjects are hyporesponsive in vivo to glucose and arginine³. Individuals affected by MODY3 mutations are typically lean with diminished insulin secretion and progressive hyperglycemia in early childhood or adolescence, there they may be misdiagnosed as type 1 diabetes (T1D)⁴. But unlike type 1 diabetes patients, although HNF1A deficient patients fail to respond to glucose, they often retain responsiveness to other stimuli, such as sulfonylureas. These patients may respond to insulin secretogogues (sulfonylureas), but often require insulin to control dysglycemia⁵. The age of onset in MODY3 patients is advanced by in utero exposure to hyperglycemia (e.g. due to maternal MODY 3)⁶. The type and position of mutations also affect the age of onset and severity of diabetes⁷.

HNF1A total knockout mice have small pancreatic islets, but it is not clear if this characteristic simply reflects the much reduced somatic size and lean mass of HNF1A knockout mice. Severely reduced beta cell mass and impaired insulin secretion is observed in MIN6 cells and in mice overexpressing a dominant-negative form of HNF1A^{9,10}. First identified as a liver-specific transcription factor, HNF1A plays a role in liver but its functions differ in in pancreatic islets and liver cells. For example, selected HNF1A target genes (Slc2a2, Pklr, and HNF4A) are down-regulated in HNF1a-deficient pancreatic islets, but not in liver⁸.

Due to limited access to patients' pancreatic beta cells, how HNF1A deficiency causes beta cell dysfunction and diabetes in human is not fully understood. There are clear differences between mouse models and affected humans. For instance, HNF1A haploinsufficiency does not cause diabetes in mice. Several studies suggest that HNF1A regulates beta cell mass, but this inference has not been definitively demonstrated; and whether HNF1A plays a role in beta cell proliferation is not clear. A central question in the pathogenesis of MODY diabetes is that why the beta cell is more sensitive to haploinsufficiency of transcription factors, such as HNF1A, than other cell types.

To answer this question, it is necessary to characterize cellular and molecular changes caused by partial loss of HNF1A function in human beta cells. We generated induced pluripotent stem (iPS) cells from four MODY3 subjects (segregating for 3 mutations) and differentiated these cells into insulin producing beta cells. HNF1A mutations caused reduced insulin production and secretion. The glucose and arginine response was also blunted in MODY3 beta cells. Global transcriptional analysis showed reduced expression of genes involved in glycolysis. Also, long term functionality of HNF1A mutant cells were reduced in a 3D culture system. The results discussed herein demonstrate that deficiency of HNF1A has broad range of impact on beta cell biology and cause multiple functional consequences.

Results

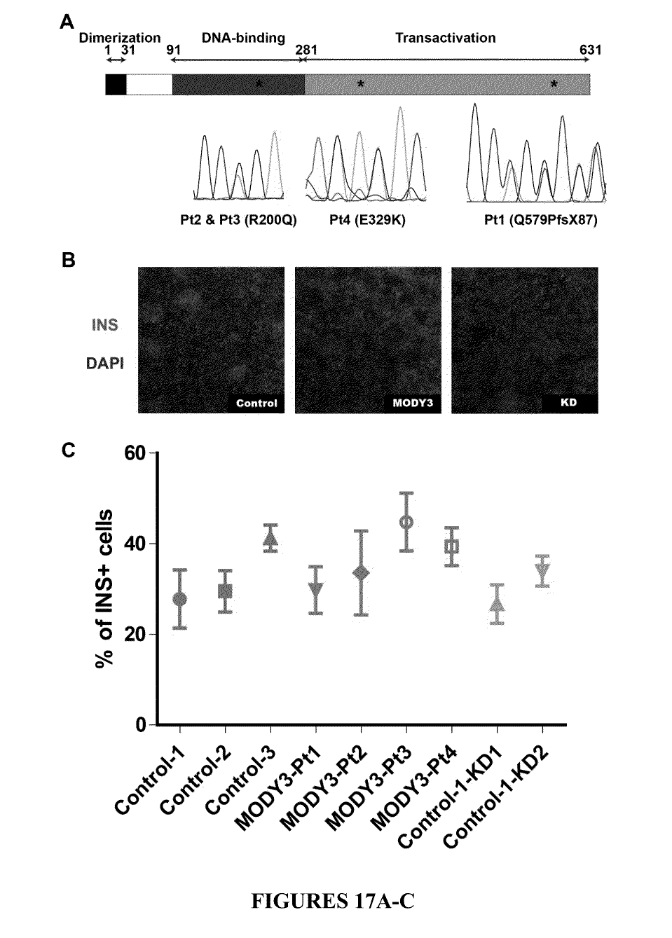

MODY3 iPS cells had normal beta cell differentiation but reduced insulin production. Skin biopsies were obtained from four MODY3 subjects and established fibroblast cell lines. One subject (MODY3-Pt1) was diagnosed with diabetes. The second and third subjects (MODY3-Pt2 and MODY3-Pt3) are diagnosed with diabetes. The fourth subject (MODY3-Pt4). All of them were non-obese and positive for measurable, but low serum C-peptide. Diabetes control was excellent on insulin or sulfonylurea-related agents. Due to their strong family histories of dominantly inherited diabetes and negative results for antibodies associated with type 1 diabetes, they underwent genetic testing. Exonic sequencing of HNF1A revealed that the MODY3-Pt1 carries an insertion mutation in transactiviation domain (FIG. 17a and Table 6). The other 3 subjects harbor missense mutations (FIG. 17a and Table 6).

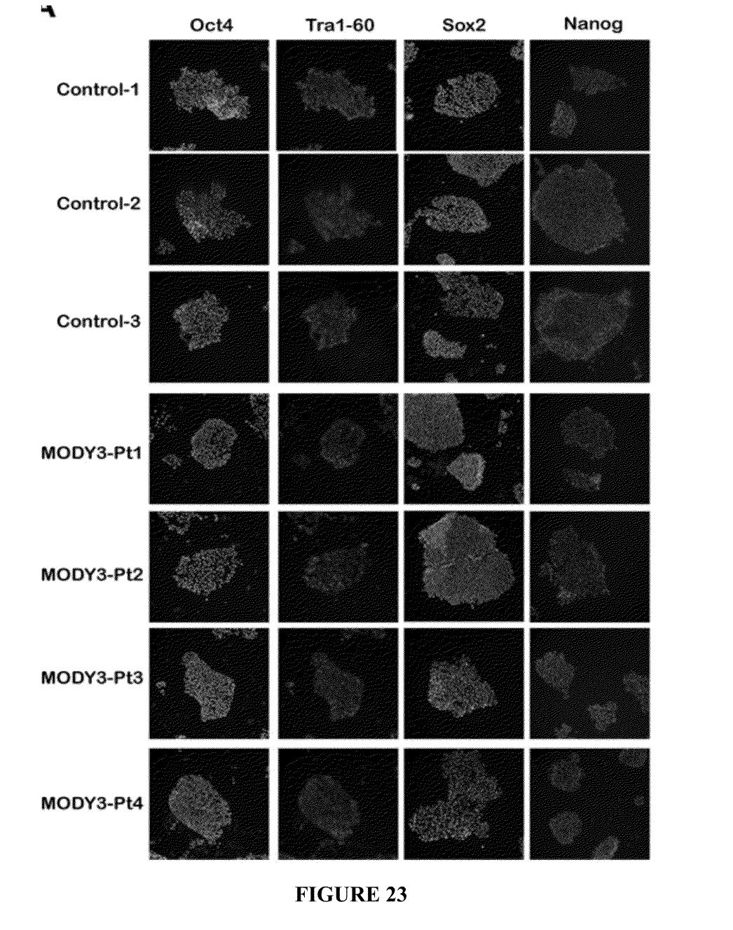

Induced pluripotent stem cell lines were generated using integration-free Sendai viruses containing Oct4, Sox2, Klf4 and c-Myc¹¹. 3 healthy control (human ES and iPS) cell lines are included in the study (Table 6). All the cell lines in this study expressed pluripotent marker genes (Oct4, Tra1-60, Sox2 and Nanog) and were able to spontaneously differentiate into 3 germ layers (FIG. 23).

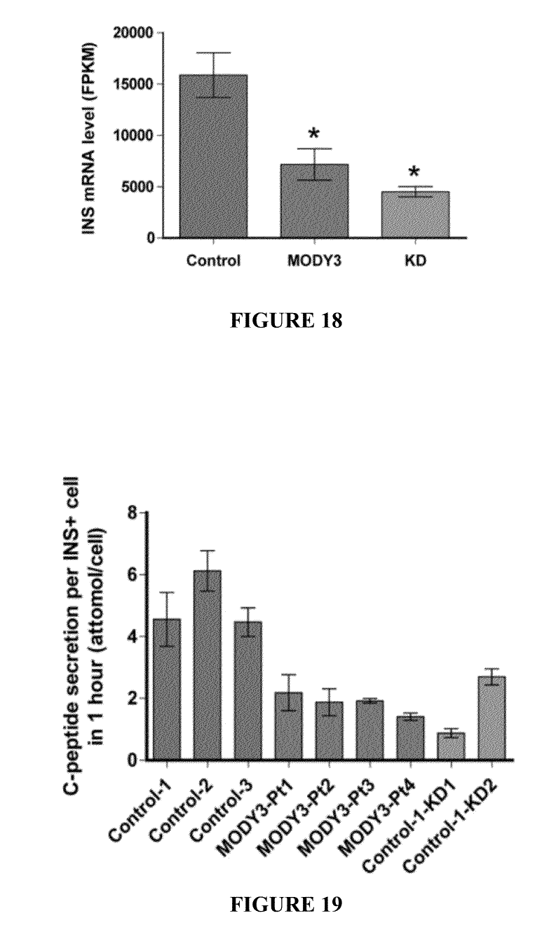

Using a previously described differentiation protocol with a few modifications^{12,13}, the stem cells were directed to pancreatic linage and derived insulin-producing beta cells. The differentiation efficiencies of the MODY3 cell lines (MODY3-Pt1 29.8%, MODY3-Pt2 33.6%, MODY3-Pt3 44.8%, and MODY3-Pt4 39.4%) were comparable to the control cell lines (Control-1 27.8%, Control-2 29.5%, and Control-3 41.2%) (FIGS. 17B and C). To control for differences in genetic background, 2 stable transgenic cell lines were generated in which HNF1A mRNA level was knocked down by shRNA. No significant differences in differentiation efficiency were noted in the KD (knockdown) cell lines (Control-1-KD1 26.7%, and Control-1-KD2 34%) (FIGS. 17B and C). Insulin (INS) mRNA levels were greatly decreased in MODY3 (55% downregulated) and KD (72% downregulated) cell lines (FIG. 18). As a consequence, the amount of insulin secreted by MODY3 (average 1.9 attomol per cell) or KD (average 1.8 attomol per cell) cells was significantly less than control cells (average 5.1 attomol per cell) (FIG. 19).

Dysfunctional Glucose and Arginine Response in MODY3 Beta Cells.

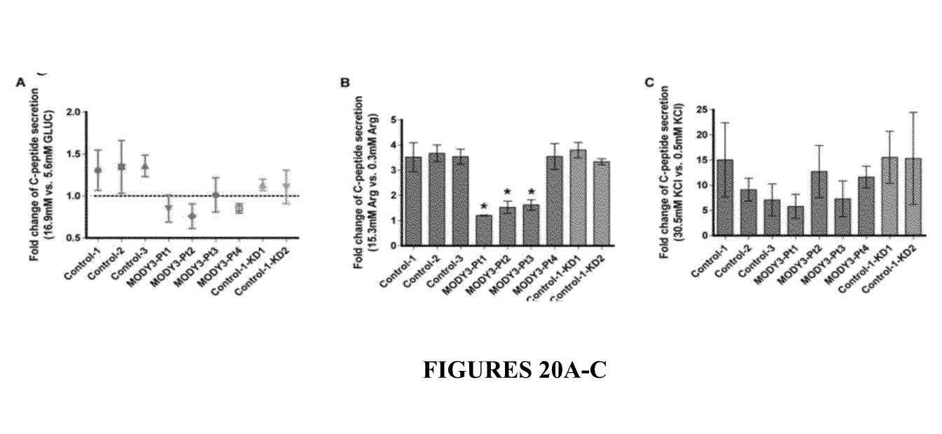

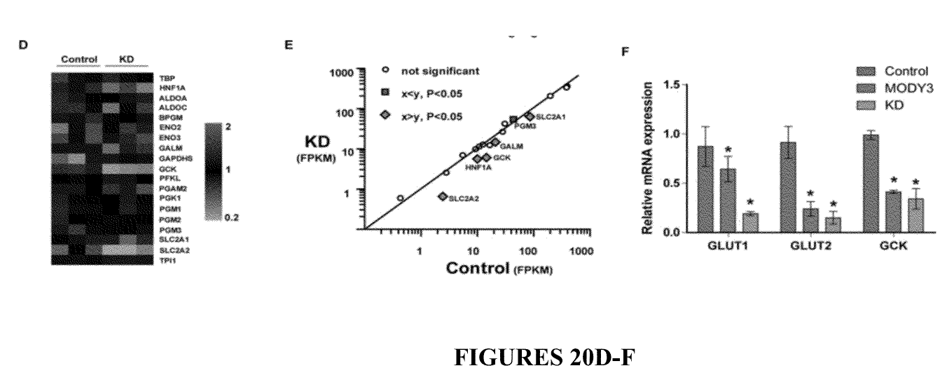

Beta cells respond to various stimuli, such as glucose, arginine or potassium, by increasing insulin secretion. Cells were challenged with 16.9 mM glucose and amount of insulin secreted were compared to the amount of insulin secreted at 5.6 mM glucose. Control cells showed a marginal response to glucose (Control-1 1.3 fold, Control-2 1.3 fold and Control-3 1.4 fold) (FIG. 20A). However, the response to glucose was blunted or significantly reduced in MODY3 (MODY3-Pt1 0.9 fold, MODY3-Pt2 0.8 fold, MODY3-Pt3 1.0 fold and MODY3-Pt4 0.9 fold) and KD (Control-1-KD1 1.1 fold and Control-1-KD2 1.1 fold) cell lines (FIG. 20A). Interestingly, the response to 15 mM arginine was severely reduced in 3 MODY3 cell lines (MODY3-Pt1 1.2 fold, MODY3-Pt2 1.5 fold and MODY3-Pt3 1.6 fold) (FIG. 20B). However, MODY3-Pt4 cells and KD cell lines (MODY3-Pt4 3.5 fold, Control-1-KD1 3.8 fold and Control-1-KD2 3.3 fold) showed arginine response comparable to control cell lines (Control-1 3.5 fold, Control-2 3.7 fold and Control-3 3.5 fold). There was no significant difference in the response to 30 mM KCl.

Decreased Expression of Glucose Transporters and Glucokinase.

To assess the molecular mechanisms for the defect in glucose response in MODY3 beta cells, the transcriptome of control and KD cells was analyzed. Although most genes in the glycolysis pathway were not affected by haploinsufficiency of HNF1A, glucose transporters and glucokinase were significantly downregulated in KD cells (FIGS. 20D and E, P<0.05). Using real time RT-PCR, mRNA levels of glucose transporter 1 (GLUT1), glucose transporter 2 (GLUT2) and glucokinase (GCK) were decreased in both MODY3 (GLUT1 36%, GLUT2 76% and GCK 59% downregulated) and KD (GLUT1 81%, GLUT2 85% and GCK 66% downregulated) cells (FIG. 20F).

Long Term Functionality was Compromised in MODY3 Beta Cells.

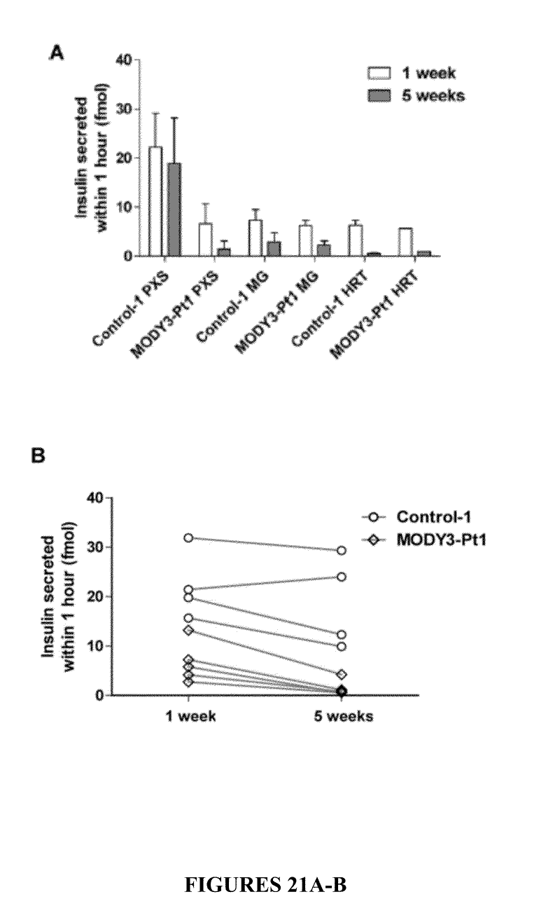

In order to maintain long term survival of beta cells in vitro, a 3D culture system was developed. Porcine pancreas was decellularized to serve as matrix for beta cells to adhere and grow. Decellularized pancreas tissue improved beta cell function and/or survival more than Matrigel or decellularized heart tissue (FIG. 21A). After 5 weeks of culture, MODY3 beta cells displayed a significantly reduced insulin release compared to control cells on pancreas matrix (FIGS. 21A and B).

MODY3 Cells Failed to Cope with Higher Level of Glucose or Fatty Acid.

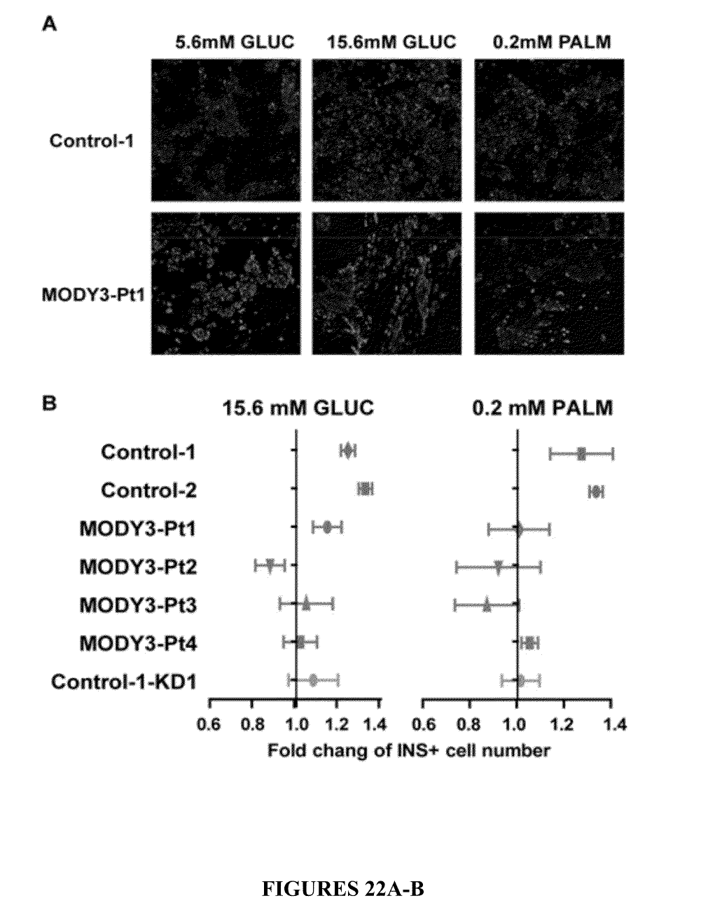

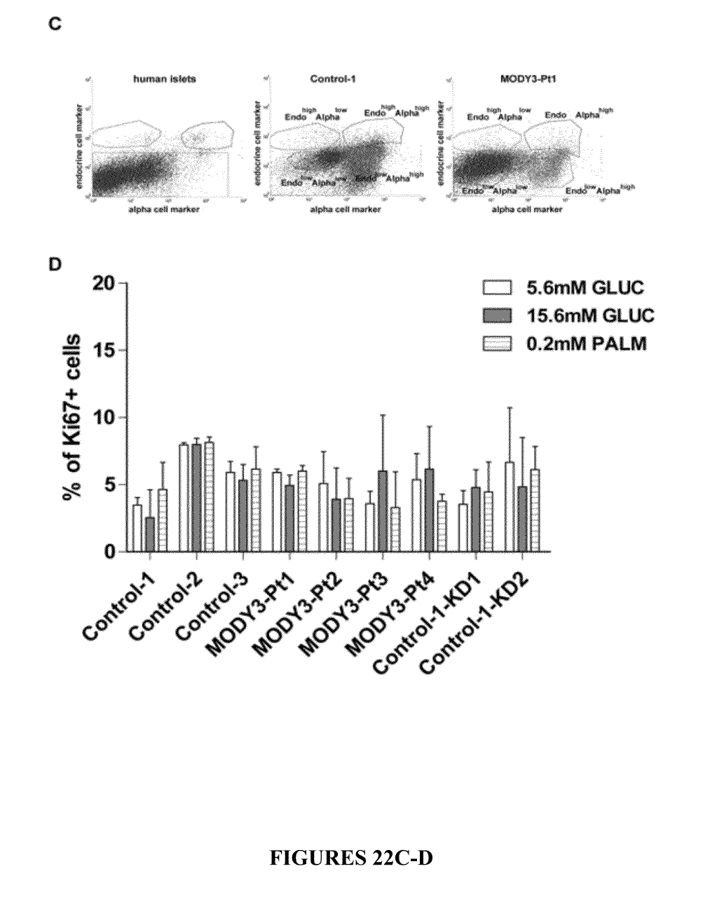

During development of human pancreas, environmental factors affect beta cell mass. MODY3 mutations carriers have an earlier age of onset if they have been exposed to diabetes in utero⁶. Cells were cultured with either 15.6 mM glucose or 0.2 mM palmitate to mimic such developmental conditions. Control stem cells responded to higher glucose or palmitate levels by producing more insulin positive cells (FIGS. 22A and B). In contrast, MODY3 stem cells failed to increase beta cell number under these culture conditions (FIGS. 22A and B). While it is reasonable to assume that the increased number of beta cells is contributed by enhanced specification from progenitors, it is also possible that beta cell replication may be elevated under high glucose or palmitate levels. To test this, beta cell population were purified using florescence activated cell sorting and cultured the beta cells with 15.6 mM glucose or 0.2 mM palmitate (FIG. 22C). Ki67 staining indicated no alteration of replication rates in either control or MODY3 cells among all culture conditions (FIG. 22D).

Discussion

The hepatocyte nuclear factor genes encode a family of transcription factors. In humans, heterozygous mutations in these genes (e.g. HNF1A, HNF4A and HNF1B) cause maturity-onset diabetes of the young (MODY), which is characterized by progressive beta-cell dysfunction. Homozygous HNF1A, HNF1B or HNF4A mutations have not been identified in humans, indicating their crucial function during development. Mice with one defective copy of the HNF1A or HNF4A gene show no diabetic phenotypes, contrary to the situation in humans. The conflicting observations in human and mouse may be due to the experimental design in mouse models but may also be attributed to the species differences.

Previously, using patient-specific beta cells from MODY2 subjects with hypomorphic mutations in the glucokinase gene, reduced glucokinase function led to decreased response to glucose in beta cells in vitro and in vivo¹³. In this study, reduced glucokinase expression was also observed in MODY3 beta cells, which can contribute to the diminished glucose response. Additional genes, including insulin and glucose transporters, were affected by partial loss of HNF1A. This can be the reason that MODY3 subjects display more severe clinical phenotypes than MODY2 subjects.

At the cellular level, HNF1A deficiency causes multiple defects in beta-cell, including insulin production and secretion, glucose and arginine response and long term functionality. Similar phenomena have been observed in beta-cell from Wolfram syndrome subjects, in which insulin production and secretion are affected due to elevated ER stress¹². In the case of MODY3 mutations, downregulated glucokinase and glucose transporters affects glucose metabolism and can cause loss of glucose response in terms of insulin secretion and differentiation from progenitors to beta cells. But blunted responses to arginine and palmitate indicate that HNF1A is affecting other target genes. The approach of generating patient-specific beta cells provides a platform to explore molecular factors that link genetic or epigenetic circumstances to diabetic phenotypes.

Methods

Research Subjects and Cell Lines.

Four MODY3 subjects and 2 healthy subjects volunteered to donate skin biopsies, which were obtained from upper arm using local anesthesia (lidocaine) and an AcuPunch biopsy kit (Acuderm Inc). Samples were coded to protect subjects' identity (Table 6). Biopsies were cut into small pieces (approximately 5×5 mm in size). 2-3 pieces of minced skin were placed next to a droplet of silicon in a well of six-well dish. A glass cover slip (22×22 mm) was placed over the biopsy pieces and silicon droplet. 5 ml of biopsy plating media were added and kept for 5 days. After that, biopsy pieces were grown in culture medium for 3-4 weeks. Biopsy plating medium was composed of DMEM, FBS, GlutaMAX, Anti-Anti, NEAA, 2-Mercaptoethanol and nucleosides (all from Invitrogen) and culture medium contained DMEM, FBS, GlutMAX and Pen-Strep (all from Invitrogen).

Generation and Characterization of Induced Pluripotent Stem Cells.

Primary fibroblasts were converted into pluripotent stem cells using CytoTune™-iPS Sendai Reprogramming Kit (Invitrogen). 50,000 fibroblast cells (between passage 2-5) were seeded in a well of six-well dish and allowed to recover overnight. Next day, the cells were infected by Sendai viruses expressing human transcription factors Oct4, Sox2, Klf4 and C-Myc mixed in fibroblast medium according to the manufacturer's instructions. Two days later, the medium was exchanged to human ES medium supplemented with the ALK5 inhibitor SB431542 (2 μM; Stemgent), the MEK inhibitor PD0325901 (0.5 μM; Stemgent), and thiazovivin (0.5 μM; Stemgent). Human ES medium contained KO-DMEM, KSR, GlutMAX, 2-Mercaptoethanol, NEAA, PenStrep and bFGF (all from Invitrogen). On day 7-10 post infection, cells were detached using TrypLE (Invitrogen) and passaged onto mouse embryonic fibroblast feeder cells. Individual colonies of induced pluripotent stem cells were manually picked between day 21-28 post infection and each iPS cell line was expanded from a single colony. All iPS cells lines were cultured on mouse embryonic fibroblast cells with human ES medium. HUES42 was chosen as a control cell line from a collection of Harvard University embryonic stem cell lines based on its robust and consistent ability to produce beta cells in vitro¹⁴. Karyotyping was performed by Cell Line Genetics Inc. For teratoma analysis, 1-2 million cells from each iPS cell line were dissociated and collected after TrypLE treatment. Cells were suspended in 0.5 ml of human ES media and then mixed with 0.5 ml matrigel (BD Biosciences). The mixture was injected subcutaneously into dorsal flanks of an immunodeficient mouse (NOD.Cg-Prkdcscid Il2rgtm1Wjl/SzJ, Stock No.:005557, The Jackson Laboratory)¹⁵. 8-12 weeks after injection, teratomas were harvested, fixed overnight with 4% paraformaldehyde and processed according to standard procedures of paraffin embedding, section and HE (hematoxylin and eosin) staining.

HNF1A Gene Knockdown.

Two lentiviruses containing shRNA sequences against HNF1A mRNA were purchased from MISSION shRNA library (Sigma). Lentivirus TRCN0000017193 targets the following sequence in the 3′UTR of HNF1A mRNA: CCGGC CTTGT TCTGT CACCA ATGTA CTCGA GTACA TTGGT GACAG AACAA GGTTTT TACTCCC ATGAAG ACGCA GAACT CGAGT TCTGC GTCTT CATGG GAGTG TTTTT (SEQ ID NO: 9). Control-1 iPS were infected by the lentiviruses according to manufacturer's instruction. One puromycin-resistant colony was selected and expanded from infection by each lentivirus.

Gene Expression Analysis.

Total RNA was isolated from cells with RNeasy Mini Kit (Qiagen). For quantitative PCR analysis, cDNA was generated using Promega RT system (Promega). Primers for qRT-PCR are listed in Table 5. RNA sequencing was performed by Columbia Genome Center. The sequencing data was analyzed using FlexArmy and Ingenuity IPA softwares.

TABLE 5
Primers for qRT-PCR

| | | SEQ |
|---|---|---|
| | | ID |
| primer | sequences | NO: |
| GCK RTPCR forward | ctgaacctcaaaccccaaac | 28 |
| GCK RTPCR reverse | tgccaggatctgctctacct | 29 |
| GLUT1 RTPCR forward | atggagcccagcagcaa | 30 |
| GLUT1 RTPCR reverse | actcctcgatcaccttctgg | 31 |
| GLUT2 RTPCR forward | catgtgccacactcacacaa | 32 |
| GLUT2 RTPCR reverse | atccaaactggaaggaaccc | 33 |

Beta Cell Differentiation.

Human ES or iPS cells were dissociated using TrypLE (Invitrogen). Cells were suspended in human ES medium containing 10 uM ROCK inhibitor (Y27632) and filtered through a 70 um cell strainer. Cells were then plated at a density of 800,000 cell/well in 12-well plates. Differentiation was started after 1 or 2 days, when cells reached 80-90% confluency. From day 1 to 3, definitive endoderm cells was generated from stem cells using STEMdiff Definitive Endoderm Kit (STEMCELL Technologies). During day 4 and 5, cells were cultured with RPMI medium (1× PenStrep, 1× GlutMAX) containing 2% FBS and KGF (50 ng/ml). During day 6 to 8, cells were cultured with DMEM-HG medium (1× PenStrep) containing and KAAD-cyclopamine (250 nM), retinoic acid (2 μM), LDN-193189 (250 nM) and 1×B27. During day 9-12, cells were cultured with DMEM-HG medium (1× PenStrep) containing exendin-4 (50 ng/ml), ALK5 inhibitor II (1 μM) and 1×B27. From day 13, cells were cultured in CMRL medium (1× PenStrep, 1× GlutMAX) containing 1×B27.

Immunostaining.

Cultured cells were washed once with PBS and fixed with 4% paraformaldehyde for 30 minutes at room temperature. Primary antibodies used in the study were as follows: mouse-anti-C-peptide (05-1109; Millipore), mouse-anti-glucagon (G2654; Sigma), rabbit-anti-OCT4 (09-0023; Stemgent), rabbit-anti-SOX2 (09-0024; Stemgent), rabbit-anti-NANOG (4903; Cell Signaling), mouse-anti-TRA1-60 (MAB4360; Millipore), rabbit-anti-Ki67 (ab15580, Abcam). Appropriate second antibodies were obtained from Invitrogen. Quantification of positively stained cells was performed using the Celigo Cytometer system (Cyntellect).

Transplantation.

After 12 days of differentiation, cells were detached using TrypLE (5 minutes at room temperature). Aliquots of 2-3 million cells were collected in eppendorf tubes, spun down and the supernatant was discarded. Then 10-15 ul matrigel (BD Biosciences) was added to each tube and mixed. Each tube of cell mixture was transplanted under the kidney capsule of an immunodeficient mouse NOD.Cg-Prkdcscid Il2rgtm1Wjl/SzJ (005557; The Jackson Laboratory) 15, following a previously described protocol 16. Three months after transplantation, human c-peptide was determined in the serum of recipient mice. An intraperitoneal glucose tolerance test was performed between 100-120 days after transplantation.

Insulin Secretion Assay.

Insulin secretion assay was perform during day 13 to 15 of differentiation, cells were washed for 1 hour in CMRL medium. Cells were then incubated in 300 μl CMRL medium containing 5.6 mM glucose for 1 hour and the medium was collected. Subsequently, 300 μl CMRL medium containing 16.9 mM glucose or 15.2 mM arginine or 30.8 mM potassium was used to treat cells for 1 hour, following which the medium was collected. For in vivo tests, mice were deprived of food overnight with ad libitum access to water. After 12-14 hours of fasting, capillary blood glucose concentrations were determined by tail bleed using an AlphaTRACK glucometer (Abbott). Venous blood samples were collected via the submandibular vein. Intraperitoneal glucose was then administered (1 mg/g body weight) and half an hour later blood samples were obtained via the submandibular vein. Blood samples were kept at room temperature for 2 hours and serum was obtained by centrifuging blood samples at 4000 rpm for 15 min. C-peptide concentrations in medium or mouse serum were measure using an ultrasensitive human C-peptide ELISA kit according to manufacturer's instructions (Mercodia).

Statistics.

Two-tailed Student's t test was used to determine statistical significance of differences between 2 groups. P values less than 0.05 were considered significant.

TABLE 6
Genetic information of cell lines included in the study.

| Cell Line | Genetic Diagnosis | Internal Reference |
|---|---|---|
| Control-1 | healthy | HUES42 |
| Control-2 | healthy | 1013A |
| Control-3 | healthy | 1016A |
| MODY3-Pt1 | Q579PfsX87 | 1056K |
| MODY3-Pt2 | R200Q | 1075A |
| MODY3-Pt3 | R200Q | 1076A |
| MODY3-Pt4 | E329K | 1124A |

### REFERENCES

1. Silander, K. et al. Genetic variation near the hepatocyte nuclear factor-4 alpha gene predicts susceptibility to type 2 diabetes. Diabetes 53, 1141-1149 (2004).
2. Saxena, R. et al. Large-scale gene-centric meta-analysis across 39 studies identifies type 2 diabetes loci. American journal of human genetics 90, 410-425 (2012).
3. Vaxillaire, M. et al. Insulin secretion and insulin sensitivity in diabetic and non-diabetic subjects with hepatic nuclear factor-1alpha (maturity-onset diabetes of the young-3) mutations. Eur J Endocrinol 141, 609-618 (1999).
4. Lambert, A. P. et al. Identifying hepatic nuclear factor 1alpha mutations in children and young adults with a clinical diagnosis of type 1 diabetes. Diabetes Care 26, 333-337 (2003).
5. Timsit, J., Bellanne-Chantelot, C., Dubois-Laforgue, D. & Velho, G. Diagnosis and management of maturity-onset diabetes of the young. Treat Endocrinol 4, 9-18 (2005).
6. Klupa, T. et al. Determinants of the development of diabetes (maturity-onset diabetes of the young-3) in carriers of HNF-1alpha mutations: evidence for parent-of-origin effect. Diabetes Care 25, 2292-2301 (2002).
7. Bellanne-Chantelot, C. et al. The type and the position of HNF1A mutation modulate age at diagnosis of diabetes in patients with maturity-onset diabetes of the young (MODY)-3. Diabetes 57, 503-508 (2008)
8. Servitja, J. M. et al. Hnf1alpha (MODY3) controls tissue-specific transcriptional programs and exerts opposed effects on cell growth in pancreatic islets and liver. Mol Cell Biol 29, 2945-2959 (2009).
9. Yamagata, K. et al. Overexpression of dominant-negative mutant hepatocyte nuclear factor-1 alpha in pancreatic beta-cells causes abnormal islet architecture with decreased expression of E-cadherin, reduced beta-cell proliferation, and diabetes. Diabetes 51, 114-123 (2002).
10. Tanizawa, Y. et al. Overexpression of dominant negative mutant hepatocyte nuclear factor (HNF)-1alpha inhibits arginine-induced insulin secretion in MIN6 cells. Diabetologia 42, 887-891 (1999).
11. Fusaki, N., Ban, H., Nishiyama, A., Saeki, K. & Hasegawa, M. Efficient induction of transgene-free human pluripotent stem cells using a vector based on Sendai virus, an RNA virus that does not integrate into the host genome. Proc Jpn Acad Ser B Phys Biol Sci 85, 348-362 (2009).
12. Shang, L. et al. Beta cell dysfunction due to increased ER stress in a stem cell model of Wolfram syndrome. Diabetes (2013).
13. Hua, H. et al. iPSC-derived beta cells model diabetes due to glucokinase deficiency. J Clin Invest 123, 3146-3153 (2013).
14. Chen, A. E. et al. Optimal timing of inner cell mass isolation increases the efficiency of human embryonic stem cell derivation and allows generation of sibling cell lines. Cell Stem Cell 4, 103-106 (2009).
15. Shultz, L. D. et al. Human lymphoid and myeloid cell development in NOD/LtSz-scid IL2R gamma null mice engrafted with mobilized human hemopoietic stem cells. J Immunol 174, 6477-6489 (2005).
16. Szot, G. L., Koudria, P. & Bluestone, J. A. Transplantation of pancreatic islets into the kidney capsule of diabetic mice. J Vis Exp, 404 (2007).

### Example 11

### Methods

Research Subjects and Cell Lines

Skin biopsies from subjects WS-1 and WS-2 were obtained at the Naomi Berrie Diabetes Center (New York), using an AcuPunch biopsy kit (Acuderm Inc). Fibroblast cells from WS-3, WS-4 and carrier were obtained from Coriell Research Institute (New Jersey), with the respective product number of GM01610, GM01611 and GM01701. All human subjects research was approved by the Columbia IRB and ESCRO committees. Research subjects signed informed consent and samples were coded. Skin biopsies were cut into 10-12 small pieces, and every 2-3 pieces were placed under a glass cover slip in a well of a six-well dish. The cover slips were adhered to the bottom of the culture dish by silicon droplets. 5 ml of biopsy plating media were added into each well. 5 days later, culture medium was used to replace the plating medium. Biopsy pieces were grown in culture medium for 3-4 weeks, with medium changes twice weekly. Biopsy plating medium contained DMEM, FBS, GlutaMAX, Anti-Anti, NEAA, 2-Mercaptoethanol and nucleosides and culture medium was composed of DMEM, FBS, GlutaMAX and Pen-Strep (all from Invitrogen).

Generation of Induced Pluripotent Stem Cells

Induced pluripotent stem cells were generated from fibroblast cells using the CytoTune™-iPS Sendai Reprogramming Kit (Invitrogen). 50,000 fibroblast cells were seeded in a well of six-well dish at passage three in fibroblast medium. Next day, Sendai viruses expressing human transcription factors Oct4, Sox2, Klf4 and C-Myc were mixed in fibroblast medium to infect fibroblast cells according to the manufacturer's instructions, 2 days later, the medium was exchanged to human ES medium supplemented by the MEK inhibitor PD0325901 (0.5 μM; Stemgent), ALK5 inhibitor SB431542 (2 μM; Stemgent), and thiazovivin (0.5 μM; Stemgent). Alternatively, iPS cells were generated with retroviral vectors (Takahashi, Tanabe et al. 2007) and tested for transgene inactivation by RT-PCR. Human ES medium contained the following: KO-DMEM, KSR, GlutaMAX, NEAA, 2-Mercaptoethanol, PenStrep and bFGF (all from Invitrogen). Individual colonies of induced pluripotent stem cells were recognized based on morphology and picked between day 21-28 post infection. Each iPS cell line was expanded from a single colony. All iPS cells lines were cultured on feeder cells with human ES medium. Karyotyping of the cells was performed by Cell Line Genetics Inc. (Wisconsin). To generate embryoid bodies, 1-2 million iPS cells of each line were detached by TrypLE (Invitrogen) treatment; cells were then collected and cultured into a low-attachment 6-well culture dish with human ES medium containing 10.mu.M ROCK inhibitor (Y27632). The next day, medium was changed to fibroblast culture medium and keep culturing for 3 weeks. Cells formed sphere morphology and were collected for immunostaining analysis. For teratoma analysis, 1-2 million cells of each iPS cell line were detached and collected by TrypLE treatment. Cells were suspended in 0.5 ml of human ES medium and mixed with 0.5 ml matrigel (BD Biosciences) and injected subcutaneously into dorsal flanks of a NOD.Cg-Prkdcscid Il2rgtm1Wjl/SzJ (NSG) mouse (Stock No. 005557, The Jackson Laboratory). 8-12 weeks after injection, teratomas were collected, fixed overnight with 4% paraformaldehyde and processed for paraffin embedding according to standard procedures. Then the samples were sectioned and HE (hematoxylin and eosin) stained.

Beta Cells Differentiation

Human ES or iPS cells were dissociated by Dispase (3-5 mins) and Accutase (5 mins, Sigma). Cells were suspended in human ES medium containing 10 μM Y27632, a ROCK inhibitor, and filtered through a 70 μm cell strainer. Then cells were seeded at a density of 800,000 cells/well in 12-well plates. After 1 or 2 days, when cells reached 80-90% confluence, differentiation was started. On Day 1: cells were briefly washed once with RPMI medium, then were treated with Activin A (100 ng/ml), Wnt3A (25 ng/ml) and 0.075 mM EGTA in RPMI medium. On day 2-3: cells were treated with Activin A (100 ng/ml) and 0.2% FBS in RPMI medium. On day 4-5: cells were treated with FGF10 (50 ng/ml), KAAD-cyclopamine (0.25 μM) and 2% FBS in RPMI medium. On day 6-8: cells were treated with FGF10 (50 ng/ml), KAAD-cyclopamine (0.25 μM), retinoic acid (2 μM) and LDN-193189 (250 nM), B27 in DMEM medium. On day 9-10: cells were treated with exendin-4 (50 ng/ml), SB431542 (2 μM) and B27 in CMRL medium. On day 11-12, cells were treated with T4 (thyroid hormone, 0.02 nM) and B27 in CMRL medium. After day 12, cells were incubated in CMRL medium with B27. Cells were analyzed between day 14 and day 16.

Immunostaining

Cells were washed once with PBS and then fixed by 4% paraformaldehyde for 30 minutes at room temperature. Embryoid bodies and mouse kidneys were fixed with 4% paraformaldehyde overnight at 4.degree. C., dehydrated using 15% (w/v) sucrose and 30% (w/v) sucrose solution and embedded in OCT compound (Tissue-Tek), and then frozen under −80° C. Cells or sections were blocked in 5% normal donkey serum for 30 minutes at room temperature. Primary antibodies used in the study were as follows: mouse-anti-SSEA4 (MAB1435; R&D systems), rabbit-anti-SOX2 (09-0024; stemgent), mouse-anti-TRA1-60 (MAB4360; Millipore), goat-anti-NANOG (AF1997; R&D systems), mouse-anti-TRA1-81 (MAB4381; Millipore), mouse-anti-OCT4 (sc-5279; Santa Cruz Biotechnology), rabbit-anti-AFP (A000829; DAKO), mouse-anti-SMA (A7607; Sigma), rabbit-anti-TUJ1 (T3952; Sigma), goat-anti-SOX17 (AF1924; R&D systems), goat-anti-PDX1 (AF2419; R&D systems), mouse-anti-C-peptide (05-1109; Millipore), rabbit-anti-glucagon (A056501; DAKO). Anti WFS1 antibody was generously provided by Dr. Urano, Fumihiko. Second antibodies were obtained from Molecular Probes (Invitrogen). Cell images were acquired by using an Olympus 1×71 fluorescence microscope and confocal microscope (ZEISS).

Unfolded Protein Response (UPR) Analysis

Wolfram and control iPSCs or fibroblasts were incubated with indicated dosages of thapsigargin (TG) or tunicamycin (TM) (Both were from Sigma) for 6 hours after an overnight starvation. 1 mM Sodium 4-phenylbutyrate (4PBA) (EMD Chemicals Inc.) was administrated one hour prior to and through TG or TM treatment. Cells were harvested and subjected to RNA and protein analysis. In vitro differentiated beta cells were treated with 10 nM TG for 12 hours, or 0.5 μg/ml TM for 6 hours with or without 1 mM 4PBA treatment one hour prior to and through TG or TM treatment. For long-term 4PBA treatment, cells were incubated with 1 mM 4PBA starting on day 9 of differentiation, when cells reached pancreatic endoderm stage, and maintained until day 15. Then cells were subjected to insulin secretion, RNA and protein analysis. RNA was isolated using RNAeasy plus kit (Qiagen). cDNA was generated by using RT kit (Promega). Primers for PCR analysis were as follows: XBP-1 for gel-imaging (Lee, Won et al.) forward 5′ GAAGCCAAGGGGAATGAAGT 3′ (SEQ ID NO:1), reverse 5′ GGGAAGGGCATTTGAAGAAC 3′ (SEQ ID NO:2); sXBP-1 for QPCR (Merquiol, Uzi et al. 2011) forward 5′ CTGAGTCCGCAGCAGGTG 3′(SEQ ID NO:3), reverse 5′ TGCCCAACAGGATATCAGACT 3′ (SEQ ID NO:4); GRP78 forward 5′ CACAGTGGTGCCTACCAAGA 3′(SEQ ID NO:5), reverse 5′ TGATTGTCTTTTGTCAGGGGT 3′ (SEQ ID NO:6); Insulin forward 5′ TTCTACACACCCAAGACCCG 3′(SEQ ID NO:7), reverse 5′ CAATGCCACGCTTCTGC 3′(SEQ ID NO:8). GRP78 protein level was determined by western blot using mouse-anti GRP78 antibody (Santa Cruz, sc-166490).

Insulin and Proinsulin Content Measurement

To determine Insulin or proinsulin content within the cell, differentiated cells were collected and lysed by M-PER protein extraction reagent (Thermo Scientific). Proinsulin and insulin contents were measured by using human proinsulin and insulin ELISA kits (Mercodia). Quantification of positively stained cells was analyzed using Celigo Cytometer system (Cyntellect), and flow cytometry analysis. To normalize insulin content to beta cell number, cultures were dissociated to single cells, and divided into three fractions: 20% of cells for cell number quantification, 40% for RNA analysis and 40% for ELISA assay to determine insulin content.

In Vitro Insulin and Glucagon Secretion Assay

Cells were cultured in 12-well dishes. After 14 days of differentiation, cells were washed for 1 hour in CMRL medium, then incubated in 300 μl CMRL medium containing 5.6 mM glucose for 1 hour and the medium was collected. After that, 300 μl CMRL medium containing 16.9 mM glucose, or 15 mM arginine, or 30 mM potassium, or 1 mM DBcAMP+16.9 mM glucose was used to treat cells for 1 hour and then the medium was collected. Human C-peptide concentration in the medium was measured by ultra-sensitive human C-peptide ELISA kit according to manufacturer's instructions (Mercodia). Glucagon levels in medium were measured by using Glucagon ELISA kit (ALPCO Diagnostics).

Transmission Electron Microscopy

Differentiated beta cells were treated with or without 10 nM TG for 12 hours, and then fixed in 2.5% glutaraldehyde in 0.1 M Sorenson's buffer (pH 7.2) for one hour. Samples were processed and imaged by Dignostic Service, Department of Pathology and Cell Biology, Columbia University. Secretory granule structure and endoplasmic reticulum (ER) morphology were visually recognized. The number of granules was determined using ImageJ software.

Transplantation and IPGTT

At 14 days of differentiation, cells were dissociated using TrypLE for 3 minutes at room temperature. 2-3 million cells were collected into an eppendorf tube, spun down and the supernatant was discarded. 10-15 μl matrigel (BD Biosciences) was mixed with the cell pellet, before transplanted into kidney capsule of a NOD.Cg-Prkdcscid Il2rgtm1Wjl/SzJ (NSG) mouse (Stock No. 005557, The Jackson Laboratory), following a previously described protocol (Szot, Koudria et al. 2007). Intraperitoneal glucose tolerance tests (IPGTT) were performed between 3 to 7 months after transplantation. Mice were deprived of food overnight (12-14 hours), but have water available. In the morning, blood glucose levels of the mice were measured by pricking the tail vein. Blood samples were collected by puncturing the submandibular vein, which locates at the backend of jaw. Then each mouse was weighed, intraperitoneal injected with a glucose solution (in saline, 1 mg/g body weight). Half an hour later, the mice were analyzed for blood glucose level and blood samples were collected again. Serum was obtained by centrifuging blood samples at 4000 rpm for 15 min. And human C-peptide concentration in the mouse serum was measured by using ultra-sensitive human C-peptide ELISA kit according to manufacturer's instructions (Mercodia). Alive nephrectomy was performed on a sub-group of receipt mice after human C-peptide was detected in the mouse serum.

### Example 12

### Wolfram iPS Cells Differentiate Normally into Beta Cells

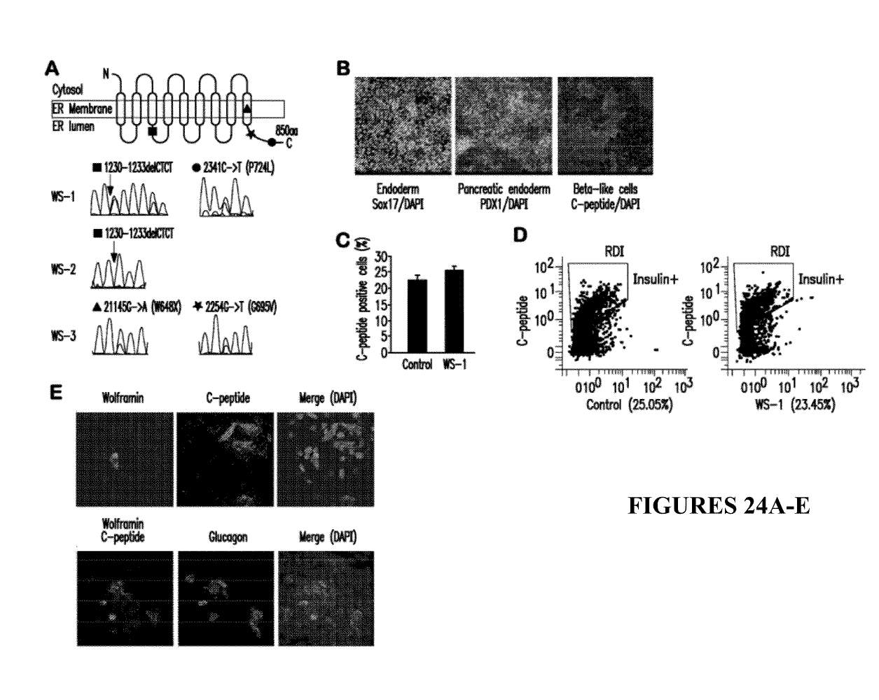

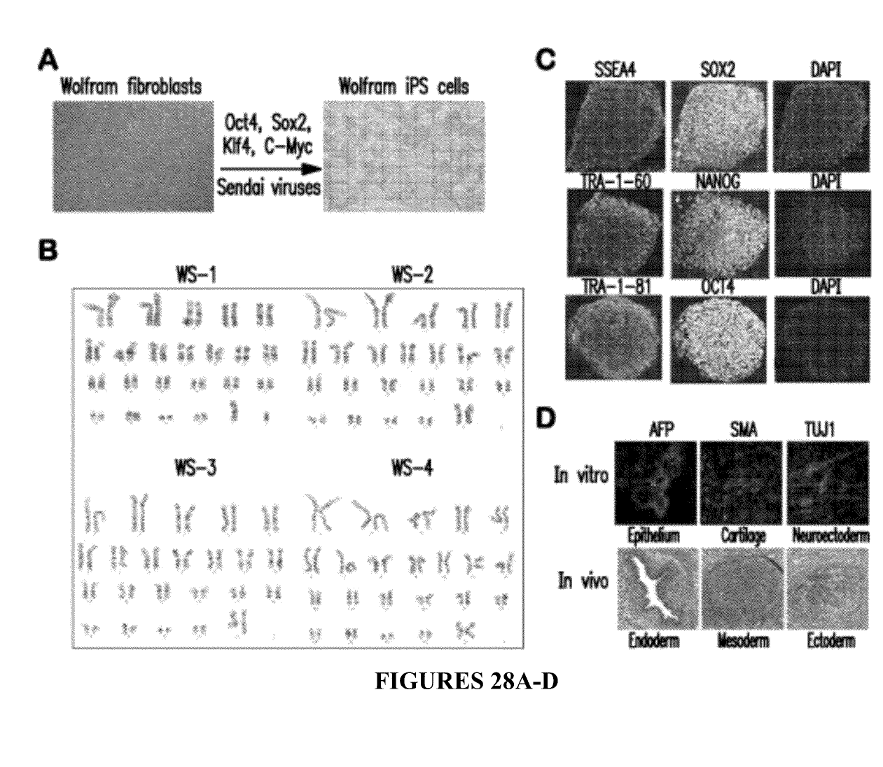

We obtained skin biopsies and established skin cell lines from two subjects affected with Wolfram syndrome, denoted: WS-1 and WS-2. Sequencing of the WFS1 locus revealed that WS-2 is homozygous for a frameshift mutation 1230-1233delCTCT (V412fsX440) (Colosimo, Guida et al. 2003), and that WS-1 is heterozygous for V412fsX440, and also carries a missense mutation P724L (Inoue, Tanizawa et al. 1998). An additional three skin cell lines were obtained from Coriell Research Institute from two siblings with Wolfram syndrome: WS-3 and WS-4, and an unaffected parent. Both WS-3 and WS-4 are heterozygous for the missense mutations W648X and G695V in the WFS1 protein (Inoue, Tanizawa et al. 1998) (FIG. 24A). All Wolfram subjects were insulin-dependent and affected by optic atrophy (Table 7). We generated induced pluripotent stem cells (iPSCs) from fibroblast cell lines using non-integrating Sendai virus vectors encoding the transcription factors Oct4, Sox2, Klf4 and c-Myc (FIG. 28A) (Fusaki, Ban et al. 2009). All iPS cell lines were karyotypically normal (FIG. 28B), expressed markers of pluripotency (FIG. 28C), and differentiated into cell types and tissues of all three germ layers in vitro and after injection into immune-compromised mice (FIG. 28D).

iPS cell lines from Wolfram and control subjects differentiated into insulin-producing cells as previously described. Differentiation efficiency of Wolfram cells was identical to controls: after 8 days of differentiation, 81.1% of total cells expressed PDX1, a marker for pancreatic endocrine progenitors, and after 13 days of differentiation, 25.6% of total cells expressed C-peptide, as determined by imaging and FACS analysis (FIG. 24B-D). To determine the expression pattern of WFS1, we performed immunostaining for WFS1 (Wolframin), insulin and glucagon. WFS1 was specifically expressed in insulin-producing cells, but not in glucagon-positive cells present in stem cell-derived islet cells from control and Wolfram subjects (FIG. 24E). Thus, stem cell-derived pancreatic cells show the expression patterns observed in the mouse pancreas, and should therefore be appropriate to study the consequences of WFS1 mutations.

TABLE 7
Information of genotypes and phenotypes of the research subjects.

| | | | Age of | Mutations in | |
|---|---|---|---|---|---|
| Cell Line | Source | Sex | Onset/Diagnosis | WFS1 gene | Remarks |
| WS-1 | Naomi | Male | 12 | 1230-1233delCTCT | Diabetes; |
| | Berrie | | | (V412fsX440), | Optic |
| | Diabetes | | | P724L | Atrophy; |
| | Center | | | | On insulin |
| WS-2 | Naomi | Female | 2 | 1230-1233delCTCT | Diabetes; |
| | Berrie | | | (V412fsX440), | Optic |
| | Diabetes | | | | Atrophy; |
| | Center | | | | On insulin |
| WS-3 | Corriell | Female | 11 | W648X, | Diabetes; |
| | Research | | | G695V | Optic |
| | Institute | | | | Atrophy; |
| | (GM01610) | | | | On insulin |
| WS-4 | Corriell | Female | 13 | W648X, | Diabetes; |
| | Research | | | G695V | Optic |
| | Institute | | | | Atrophy; |
| | (GM01611) | | | | On insulin |
| Carrier | Corriell | Male | Not affected | G695V | On insulin; |
| | Research | | | | Non- |
| | Institute | | | | diabetic; |
| | (GM01701) | | | | Father of |
| | | | | | WS-3 and |
| | | | | | WS-4 |
| Control | Harvard | Male | Not affected | Normal | Non- |
| (HUES42) | University | | | | diabetic |
| Control-2 | Naomi | Male | Not affected | Normal | Non- |
| (iPSC) | Berrie | | | | diabetic |
| | Diabetes | | | | |
| | Center | | | | |

### Example 13

### Activated UPR Reduces Insulin Synthesis in Wolfram Beta Cells

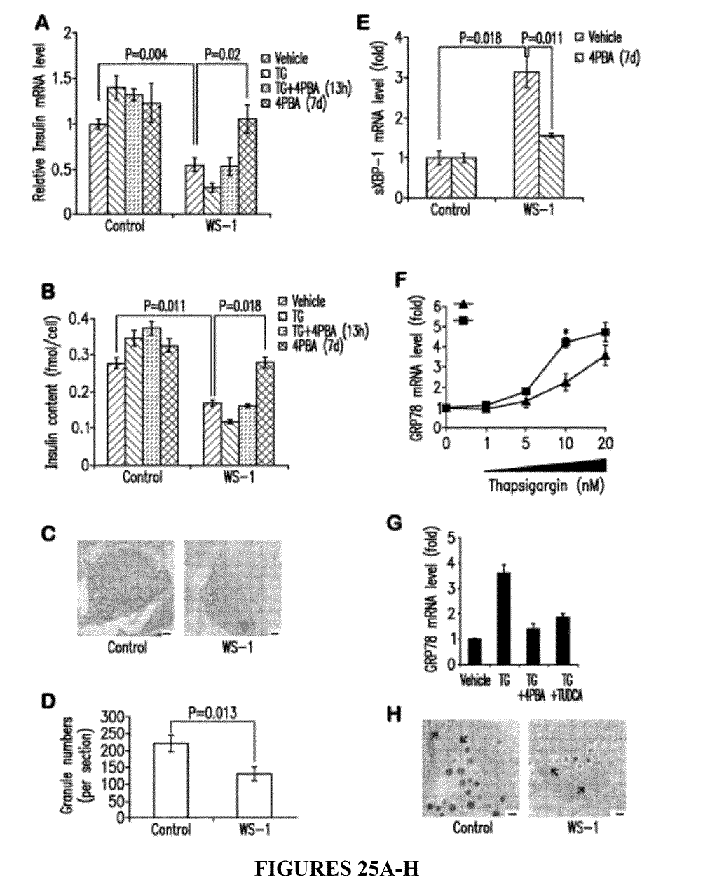

To investigate how WFS1 mutations affect beta-cell function, we first quantified insulin mRNA and protein content in Wolfram, and control stem cell-derived beta cells. To normalize insulin content to beta cell number, cultures were dissociated to single cells, and divided into three fractions to determine cell number, RNA level and insulin content. The insulin mRNA was normalized to TBP (TATA-binding protein) mRNA and to the percentage of insulin-positive cells in each sample. Similarly, insulin content was normalized to the total number of insulin-positive cells. WFS1 deficiency was associated with a 45% reduction in insulin mRNA levels compared to controls (FIG. 25A), and a 40% decrease of insulin protein content (FIG. 25B). This decrease was also reflected in the number of secretory granules imaged by transmission electron microscopy. Differentiated beta cells from unaffected individual contained abundant secretory granules. In contrast, a 41% reduction in the number of secretory granules was observed in Wolfram-derived beta cells (FIGS. 25C and D). To determine whether the lower insulin content in Wolfram beta cells was caused by increased insulin secretion, or by lower insulin synthesis, we determined the 1 hour secretion rate of C-peptide in response to 5.6 mM glucose. The rates were 0.00316 and 0.00384 fmol per hour for Wolfram and control cells, respectively. These rates are equal to 1.9% and 1.4% of insulin content in the Wolfram and control beta cells, respectively. Therefore, the reduced insulin content in Wolfram beta cells is not likely due to increased insulin secretion, but to lower rates of insulin synthesis.

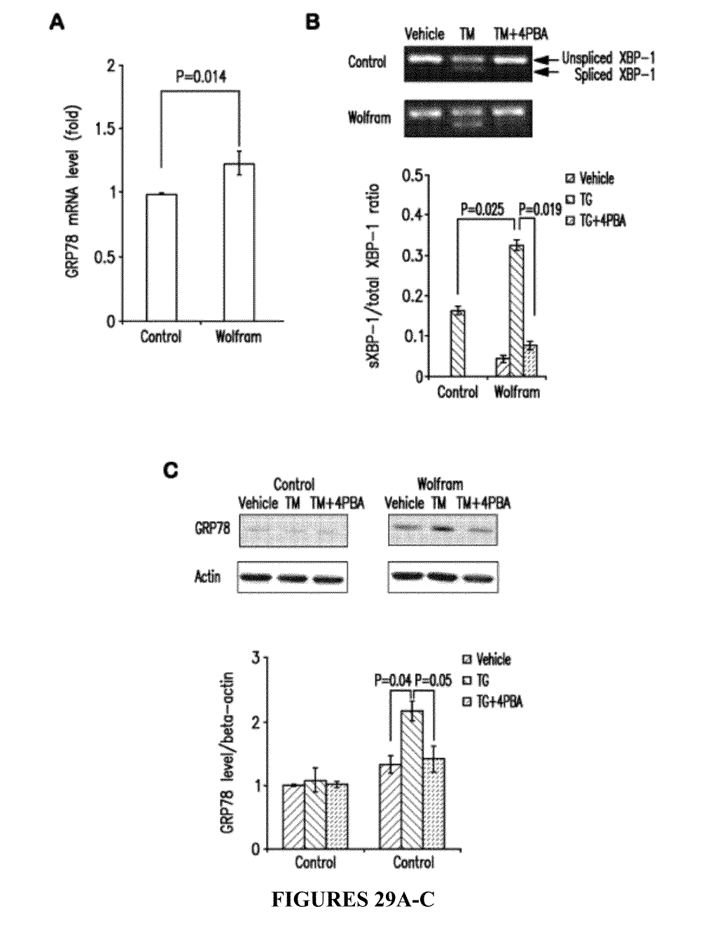

To determine the cause of the decreased insulin synthesis, we investigated the expression of components of the unfolded protein response (UPR) in Wolfram cells. IRE-1 kinase/ribonuclease and PERK, a kinase phosphorylating initiation factor 2a, sense increases in unfolded protein, and impose a state of translational repression in response to an increase in unfolded proteins. IRE-1alpha activity is reflected in the splicing of XBP-1 mRNA, allowing translation of a functional XPB-1 transcription factor (Iwawaki, Hosoda et al. 2001; Kimata, Ishiwata-Kimata et al. 2007). Long-term exposure of rat INS-1 cells to high glucose concentrations causes hyper-activation of IRE1, which leads to decreased insulin gene expression (Lipson, Fonseca et al. 2006). In beta cell cultures, iPS cells and fibroblasts, we found that levels of spliced XBP-1 mRNA, GRP78 mRNA and protein, were increased in Wolfram subject samples in comparison to controls (FIG. 25E, FIG. 29A-C). These differences between control and Wolfram cells were further enhanced by the imposition of experimental ER stress. In stem cells, thapsigargin (TG) caused a dose-dependent increase in GRP78 mRNA level and 6 hour of 10 nM TG treatment caused a greater increase of GRP78 mRNA in Wolfram cells than in control cells (4 fold versus 2 fold (FIG. 25F). Thapsigargin (TG) induces ER stress by disrupting intracellular calcium homeostasis through the inhibition of the Ca²⁺-ATPase responsible for Ca²⁺ accumulation in ER (Wong, Brostrom et al. 1993). Importantly, chemical chaperones sodium 4-phenylbutyrate (4PBA) (de Almeida, Picarote et al. 2007; Yam, Gaplovska-Kysela et al. 2007) and tauroursodeoxycholate (TUDCA) (Berger and Haller 2011) effectively reduced GRP78 mRNA levels in Wolfram cells treated with TG (FIG. 25G). Similarly, another ER stress inducer, tunicamycin (TM), which activates UPR by inhibiting N-linked glycosylation (Kozutsumi, Segal et al. 1988), induced a stronger UPR response in Wolfram iPS and fibroblast cells than in control cells. Spliced XBP-1 (sXBP-1) mRNA (FIG. 29B) and GRP78 protein levels (FIG. 29C) were higher in Wolfram cells. Both sXBP-1 and GRP78 were reduced by the addition of 4PBA.

If UPR signaling were responsible for the reduced insulin synthesis in Wolfram beta cells, elevated ER stress should further reduce insulin production, while reducing ER stress would protect insulin content. To test this inference, we experimentally increased or reduced UPR activation using TG or 4PBA in beta cell cultures. When Wolfram beta cells were generated in the presence of 4PBA from day 9 to day 15 of differentiation, sXBP-1 mRNA levels were reduced by 50% (FIG. 25E). Strikingly, this long-term incubation with 4PBA increased insulin mRNA in Wolfram cells by 1.9-fold and insulin content by 1.7-fold, to levels comparable to those in control cells without 4PBA (FIGS. 25A and B). When control cells were exposed to the same 7 d treatment of 4PBA during beta-cell differentiation, a moderate increase (1.2 fold) of insulin production was also observed (FIGS. 25A and B). Exposing Wolfram beta cells to the ER stressor TG had the opposite effect: production of insulin was reduced by 46% at the mRNA level and 31% at the protein level, while control cells were unaffected (FIGS. 25A and B). Experimentally induced ER stress also affected ER morphology: the ER was greatly dilated in Wolfram beta cells in the presence of TG, while control cells remained unaffected (FIG. 25H). These results suggest that WFS1 acts in beta cells to maintain ER function under protein folding stress.

### Example 14

### Normal Stimulated Insulin Secretion in WFS1 Mutant Cells

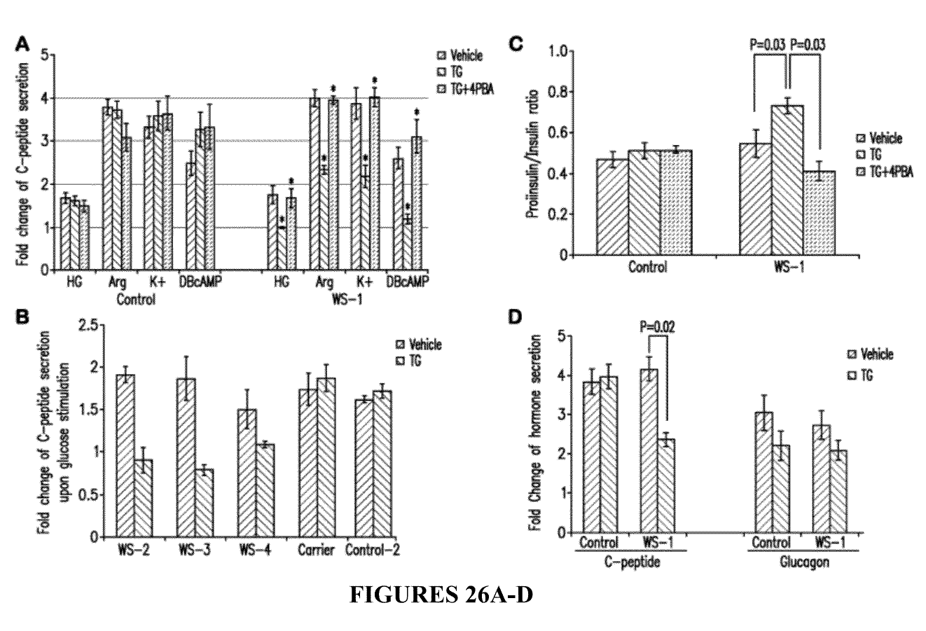

To test the ability of Wolfram beta cells to secrete insulin, we exposed them to various secretagogues, including glucose, arginine, potassium and the cAMP analog, dibutyl cAMP (DBcAMP). Our expectation was that the response to different secretagogues would reveal whether WFS1 was involved in specific steps of the cellular signals leading to insulin secretion as has been suggested by others (Fonseca, Urano et al. 2012). Glucose stimulates insulin secretion by ATP generation, resulting in the closing of the ATP sensitive potassium channel and reduction of potassium efflux, which stimulates Ca²⁺ influx and triggers exocytosis of insulin granules (Lebrun, Malaisse et al. 1982; Miki, Nagashima et al. 1998). Arginine induces insulin secretion by triggering Ca²⁺ influx, without reducing potassium efflux (Henquin and Meissner 1981; Herchuelz, Lebrun et al. 1984). cAMP influences insulin secretion by enhancing Ca⁺ influx and mobilizing insulin granules (Malaisse and Malaisse-Lagae 1984; Seino and Shibasaki 2005). And finally, extracellular potassium bypasses these upstream events by directly depolarizing the plasma membrane, resulting in the release of insulin granules (Matthews and O'Connor 1979; Matthews and Shotton 1984). To assess insulin secretion in response to glucose, we incubated cells to medium containing 5.6 mM glucose for 1 hour, followed by medium containing 16.9 mM glucose for 1 hour. Controls and heterozygous carrier beta cells showed a 1.6 to 1.7-fold higher level of C-peptide in the medium after addition of 16.9 mM glucose. A similar increase of 1.5 to 1.9 fold was seen in all four WFS1 mutant cells (FIGS. 26A and B). We further tested insulin secretion in response to arginine, potassium, and DBcAMP. Independent of the genotype and the secretagogue, a 2-4 fold increase in C-peptide secretion was observed in both control and WFS1 mutant cells (FIG. 26A). Therefore, although Wolfram beta cells showed reduced insulin content, they displayed a normal functional response to secretagogues acting at different points in metabolic sensing and insulin release.

### Example 15

### Wolframin Preserves Stimulated Insulin Secretion Under Elevated ER Stress

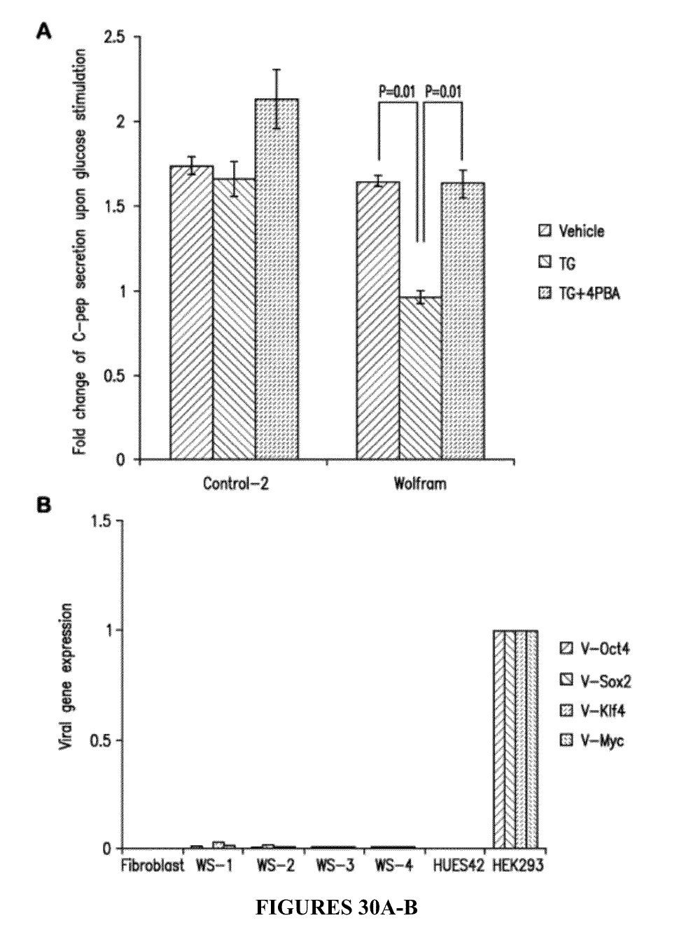

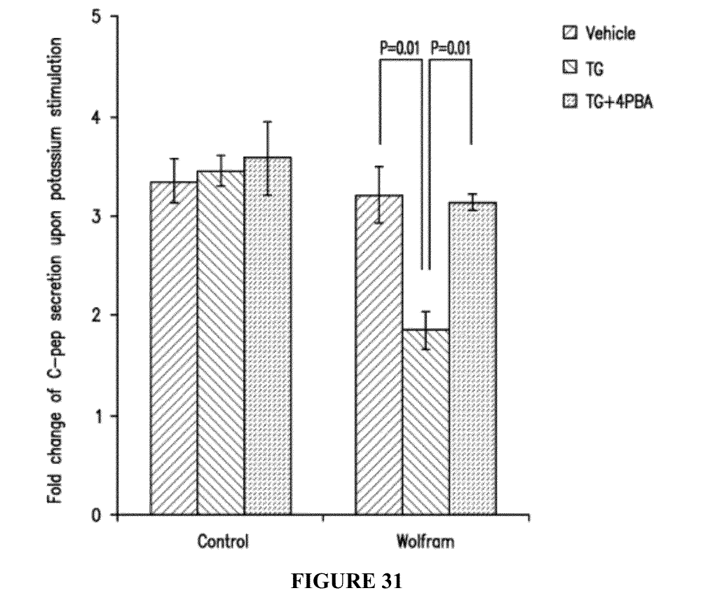

To determine whether WFS1 deficiency affected stimulated insulin secretion under ER stress, we again determined insulin secretion in response to different secretagogues. When thapsigargin (TG) treated cells were exposed to high ambient glucose (16.9 mM), Wolfram cells failed to increase insulin secretion, while control beta cells increased insulin output by 1.6 fold. Incubation with 4PBA prevented these detrimental effects of TG on Wolfram beta cells (FIG. 26A). The reduction in stimulated insulin secretion by TG was seen with all secretagogues tested, independent of their mechanism of action. When Wolfram beta cells were treated with TG, the fold increase of C-peptide in the medium decreased from 4.0 to 2.3 fold in response to arginine; and insulin-secretion in response to potassium dropped from 3.9 fold to 2.2 fold; the response to DBcAMP declined from 2.6 to 1.2 fold. Independent of the secretagogue used for stimulation, 4PBA prevented the decrease in insulin secretion upon application of ER stressor (FIG. 26A). We also determined that the sensitivity to ER stress in Wolfram cells was not cell line dependent, or dependent on the method used to generate iPS cells. A reduction in stimulated insulin secretion was observed for beta cells generated from all four Wolfram subjects, but not for a carrier and another control iPSC line (FIG. 26B). The reduced beta cell function was seen with iPS cells independent of the method of generation (FIGS. 30A and B) and also did not depend on the ER stressor: a reduction in insulin secretion was also observed in tunicamycin (TM)-treated Wolfram beta cells upon potassium stimulation (FIG. 31).

To determine whether the decreased responsiveness to secretagogues might be related to insulin processing/packaging, we determined the ratio of proinsulin/insulin in beta cells (FIG. 26C). We found that the proinsulin/insulin ratio in Wolfram beta cells was .about.0.55, similar to control cells (˜0.47). However, when cells were challenged with TG, the proinsulin to insulin ratio in the Wolfram beta cells increased to 0.73, which was significantly higher than that in control beta cells (0.51, P=0.03). 4PBA treatment restored normal insulin processing in TG-exposed Wolfram beta cells.

Because of the specific expression of WFS1 in beta cells (FIG. 24E), but not in glucagon expressing cells, we would expect that mutations differentially affect beta cells and alpha cells. We differentiated Wolfram cells into clusters containing both glucagon expressing and insulin expressing cells (FIG. 24E) and stimulated these cells with arginine. As arginine stimulates both endocrine cell types, we were able to determine stimulated hormone secretion in the same experiment, with and without TG treatment. TG treatment reduced stimulated glucagon secretion in control and WFS1 cells by 28% and 24% respectively. In contrast, the reduction of stimulated insulin secretion only occurred in WFS1 mutant cells (−3% versus 43%) (FIG. 26D).

### Example 16

### Declining Stimulated Insulin Secretion of Wolfram Beta Cells In Vivo

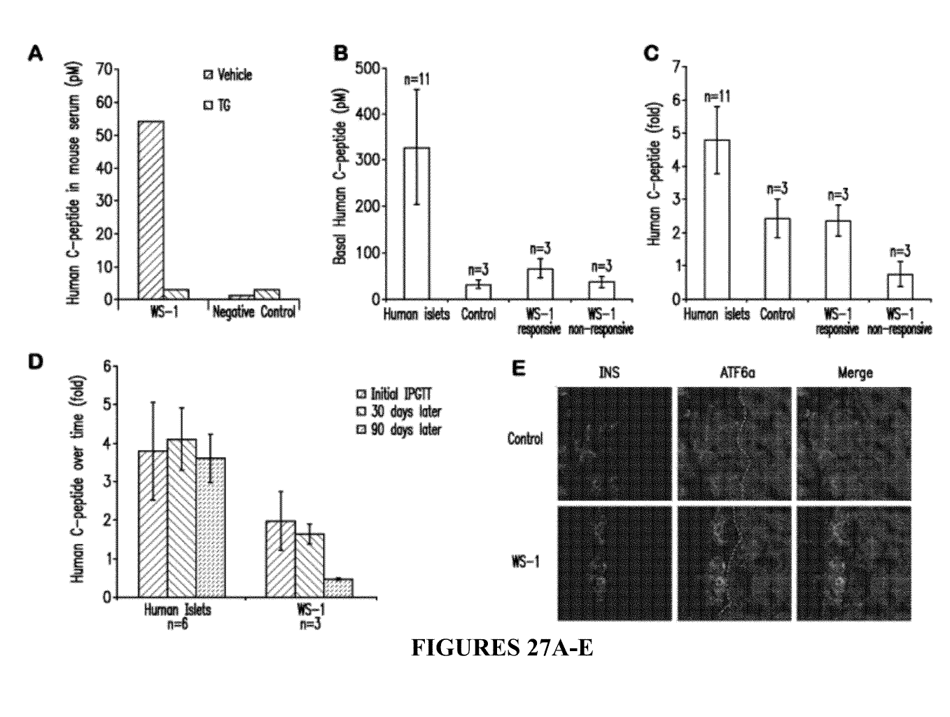

A potential limitation of an in vitro model is that it may not fully recapitulate all relevant characteristics due to the lack of a physiological (in vivo) environment that allows functional testing over a longer time period. After 14 days of in vitro differentiation, 2-3 million pancreatic endodermal cells were transplanted into the kidney capsule of immune-deficient mice. Human C-peptide was first detected 13 weeks post transplantation in the serum of mice transplanted with Wolfram and control cells in all, (6/6) mice. C-peptide originated from the graft, as human C-peptide became undetectable 2 days after the removal of the kidney containing the transplanted cells (FIG. 27A). All mice with Wolfram grafts had basal serum human C-peptide concentrations comparable to the control group (FIG. 27B). To determine the functional capacity of these grafts, intraperitoneal glucose tolerance tests (IPGTT) were performed. In 11 mice transplanted with human islets, C-peptide concentrations increased on average 4.78-fold (1.06-11.28 fold). Mice transplanted with control HUES-derived cells (n=3) showed a mean 2.43-fold increase (1.75-2.87 fold) of human C-peptide in serum. Mice transplanted with Wolfram-derived cells exhibited heterogeneous responses: 3 out of 6 mice showed a mean 2.35-fold increase of human C-peptide serum concentration, and the other 3 had no response to glucose (averaging a 0.75-fold reduction of human C-peptide) (FIG. 27C). Notably, grafts of Wolfram-derived cells, but not human islet controls lost their ability to respond to glucose within 90 days after the initial IPGTT test; fold induction remained 3.60 fold for human islets, and decreased below 1 for the Wolfram cells (FIG. 27D). Interestingly, although Wolfram implants lost their response to glucose, their basal secretion of human C-peptide remained stable (Initial average basal C-peptide was 58.18 pM, 30 days after was 55.71 pM and 90 days after was 95.44 pM). To determine the cause of impaired glucose-stimulated insulin secretion in Wolfram implants, one control and one Wolfram graft was isolated for histological analysis for the beta cell clusters. Although the insulin staining intensity of the Wolfram beta cells appeared similar to controls, a higher expression of ER stress marker, ATF6.alpha. was observed in transplanted graft containing Wolfram cells compared to control cells (FIG. 27E).

### Example 17

### Results

A Stem Cell Model of ER Stress Induced Diabetes

Here we report a stem-cell based model of Wolfram syndrome, a fatal disorder characterized by diabetes with selective beta cell loss in the pancreas, as well as severe neuropathic phenotypes. Our model is remarkably faithful in recapitulating the beta cell physiology, and associated phenotypes seen in Wolfram syndrome. We found specific expression of WFS1 in beta cells and functional phenotypes ranging from reduced insulin content at low levels of ER stress, to a dilated endoplasmic reticulum, defective insulin processing, and a failure to secrete insulin in response to canonical stimuli at elevated levels of ER stress. Specific expression of WFS1 in beta cells has also been observed in mouse and human islets, and the phenotypes described are consistent with those reported in the mouse. For instance, a similar dilation of the ER and elevated ER stress markers have also been observed in a Wfs1 mutant mouse.

Despite the availability of a Wfs1 mutant mouse, the mechanisms how Wolframin mutations result in beta cell dysfunction and diabetes have remained unclear. Several models have been proposed for the role of WFS1 in beta cells, including generation of cAMP upon glucose stimulation, calcium homeostasis in the ER, a role in insulin processing and or as a negative regulator of the unfolded protein response by inhibiting ATF6 induced transcription. Our results are consistent with a primary role of WFS1 in protecting beta cells from protein folding stress and ER dysfunction. Beta cells of control subjects were resistant to experimentally induced ER stress, but rapidly lost functionality in the absence of WFS1. At the same concentrations of ER stress effectors, glucagon producing alpha cells of both control and wolfram mutant genotypes were affected to an equal and smaller extent than beta cells. We and others found that all three major pathways of UPR signaling are activated in the absence of WFS1, including PERK, IRE1 and ATF6, suggests that WFS1 primarily acts upstream of UPR signaling and not by regulating the activity of a particular UPR pathway. Under normal physiological conditions, the absence of WFS1 in beta cells results in elevated UPR signaling and a reduction of insulin synthesis. A further increase in ER stress causes beta cell failure by affecting insulin processing and stimulated insulin secretion. These phenotypes observed in vitro likely reflect beta cell failure after transplantation in vivo: glucose stimulated insulin secretion was initially present in some of the mice transplanted with human Wolfram cells, but over a time period of 90 days, the ability to increase insulin secretion in response to glucose was lost, and ER stress markers were increased in comparison to controls.

Stem Cell Model to Identify Compounds that Protect Beta Cells and Enhance their Function

Our model of Wolfram syndrome provides a platform for drug discovery and testing. We found that the chemical chaperone 4PBA is effective at reverting ER stress associated phenotypes in beta cells. This molecule or compounds with similar activity may be useful in preventing or delaying beta-cell dysfunction in Wolfram syndrome, and possibly other forms of diabetes.

Our results using Wolfram syndrome cells show that these cells reflect the phenotype of the affected subject. In addition to being relevant for Wolfram syndrome, our observations are likely relevant for other forms of diabetes. Unresolved ER stress may result in an inability of beta cells to secrete insulin in response to nutrients, and eventually beta cell death in all forms of diabetes. Beta cells of T2D and T1D subjects may have greater intrinsic ability to increase insulin synthesis in response to metabolic demand than Wolfram cells, but likely encounter a similar mismatch between metabolic demand and the ability to increase insulin production, resulting in elevated UPR signaling. In T1D, a decreasing number of beta cells endeavor to meet metabolic demand for insulin, and in most instances of T2D, the demand for insulin is increased because of peripheral insulin resistance. Increased expression of ER stress marker genes has been observed in the islets of type I diabetic mice and humans. Activation of ER stress associated genes (i.e. PERK and GRP78) has also been observed in the liver of mouse models of T2D and a higher susceptibility to ER stress induced by metabolic perturbations was observed in isolated islets in T2D patients. Reducing the demand for insulin by intensive insulin therapy improves endogenous beta cell function in T1D, and improving insulin sensitivity by PPARg inhibitors or by weight loss meliorates T2D, in part because beta cell function is improved. Common alleles in WFS1 are associated with increased diabetes risk. In the aggregate these earlier studies and those reported here support the concept of a role for ER stress in mediating aspects of the susceptibility and response of beta cells to failure in the context of diabetes.

Stem cell models of diabetes can be used for drug discovery and drug screening. We have identified two drugs, 4-PBA and TUDCA that reduce the activity of ER stress pathways, and improve beta cell function in a stem cell model of Wolfram syndrome. Our results suggest that the most effective intervention to restore some beta cell function in diabetes would be to reduce the demand for insulin (reduce the requirement for insulin synthesis), and at the same time to facilitate protein folding using chemical chaperones to reduce endoplasmic reticulum stress.

Although the invention has been described with reference to the above examples, it will be understood that modifications and variations are encompassed within the spirit and scope of the invention. Accordingly, the invention is limited only by the following claims.

What is claimed is:

1. A method for generating a beta cell from a stem cell or an induced pluripotent stem cell, the method comprising:
(a) contacting the cells with a first culture medium, wherein the first culture medium is an RPMI medium comprising 1× Pen-Strep and 1× Glutamax and wherein the first culture medium further comprises Activin A, Wnt3A and Ethylene glycol-bis(2-aminoethylether)-N,N,N′,N′-tetraacetic acid,
(b) contacting the cells with a second culture medium, wherein the second culture medium is an RPMI medium comprising 1× Pen-Strep and 1× Glutamax and wherein the second culture medium further comprises Activin A protein and FBS in RPMI medium,
(c) contacting the cells with a third culture medium, wherein the third culture medium is an RPMI medium comprising 1× Pen-Strep and 1× Glutamax and wherein the third culture medium further comprises containing human FGF10 protein, KAAD-cyclopamine and FBS in RPMI medium,
(d) contacting the cells with a fourth culture medium, wherein the fourth culture medium is an DMEM high glucose medium comprising 1× Pen-Strep and 1× Glutamax and wherein the fourth culture medium further comprises FGF 10, KAAD-cyclopamine, retinoic acid, LDN-193189 and 1×B27,
(e) contacting the cells with a fifth culture medium, wherein the fifth culture medium is a CMRL medium comprising 1× Pen-Strep and 1× Glutamax and wherein the fourth culture medium further comprises exedin-4, SB431542 and 1×B27, and
(f) contacting the cells with a sixth culture medium, wherein the sixth culture medium is a CMRL medium comprising 1× Pen-Strep and 1× Glutamax and wherein the sixth culture medium further comprises 4-(4-Hydroxy-3,5-diiodophenoxy)-3,5-diiodobenzeneacetic acid and 1×B27.

2. The method of claim 1, wherein the beta cell is a pancreatic progenitor cell, an insulin producing cell or an endoderm cell.

3. The method of claim 1, wherein the stem cell is an embryonic stem cell.

4. The method of claim 1, wherein the cells are mammalian cells

5. The method claim 1, wherein the cells are human cells.

6. The method of claim 1, wherein any of the first, second, third, fourth, fifth or sixth culture media further comprise EGTA.

7. The method of claim 1, wherein the concentration of Activin A in the first culture medium is about 100 ng/ml, wherein the concentration of Wnt3A in the first culture medium is about 25 ng/ml and wherein the concentration of Ethylene glycol-bis(2-aminoethylether)-N,N,N′,N′-tetraacetic acid in the first culture medium is about 0.15 mM.

8. The method of claim 1, wherein the cells are cultured in the first culture medium for about 24 hours.

9. The method of claim 1, wherein the concentration of Activin A in the second culture medium is about 100 ng/ml and wherein the concentration of FBS in the second culture medium is about 0.2% FBS by volume.

10. The method of claim 1, wherein the cells are cultured in the second culture medium for about 24 hours.

11. The method of claim 1, wherein the concentration of FGF10 in the third culture medium is about 50 ng/ml, wherein the concentration of KAAD-cyclopamine in the third culture medium is about 0.25 uM, and wherein the concentration of FBS in the third culture medium is about 2% FBS by volume.

12. The method of claim 1, wherein the cells are cultured in the third culture medium for about 48 hours.

13. The method of claim 1, wherein the concentration of FGF10 in the fourth culture medium is about 50 ng/ml, wherein the concentration of KAAD-cyclopamine in the fourth culture medium is about 0.25 uM, wherein the concentration of retinoic acid in the fourth culture medium is about 2 uM, and wherein the concentration of LDN-193189 in the fourth culture medium is about 250 nM.

14. The method of claim 1, wherein the cells are cultured in the fourth culture medium for about 72 hours.

15. The method of claim 1, wherein the concentration of exedin-4 in the fifth culture medium is about 50 ng/ml, and wherein the concentration of SB431542 in the fifth culture medium is about 2 uM.

16. The method of claim 1, wherein the cells are cultured in the fifth culture medium for about 48 hours.

17. The method of claim 1, wherein the concentration of 4-(4-Hydroxy-3,5-diiodophenoxy)-3,5-diiodobenzeneacetic acid in the sixth culture medium is about 20 pM.

18. The method of claim 1, wherein the cells are cultured in the sixth culture medium for about 48 hours.

19. The method of claim 1, wherein any of the first, second, third, fourth, fifth or sixth culture media are replaced with fresh corresponding media prior to contacting the cells with media having a different composition.

20. The method of claim 1, further comprising a step of maintaining the cells after step (f) in a CMRL medium comprising 1×B27 and 1× Glutamax.

21. The method of claim 1, wherein any of the first, second, third, fourth, fifth or sixth culture media further comprise an antibiotic.

22. The method of claim 21, wherein the antibiotic is Pen-Strep.

23. The method of claim 1, wherein the induced pluripotent cells are generated by
(a) obtaining a source cell by taking a skin biopsy from a mammal (e.g. a mouse or a human),
(b) establishing a fibroblast cell line from the skin biopsy, and
(c) infecting the fibroblast cell line with a retrovirus or a Sendai virus capable of directing expression of human transcription factors Oct4, Sox2, Klf4 and C-Myc in the fibroblast cell line.

24. The method of claim 1, wherein the stem cell or the induced pluripotent stem cell is from a mammal having, or at risk of having, type I diabetes, type II diabetes, pre-diabetes or any combination thereof.

25. The method of claim 1, wherein the stem cell or an induced pluripotent stem cell comprises a diabetes-associated mutation.

26. The method of claim 25, wherein the diabetes-associated mutation is a glucokinase G299R mutation.

27. A method for treating a mammal having, or at risk of having, type I diabetes, type II diabetes, pre-diabetes or any combination thereof, the method comprising administering to the mammal a pancreatic progenitor cell, an insulin producing cell or an endoderm cell of claim 1.
